(19) United States
(12) Patent Application Publication
(10) Pub. No.: US 2022/0282265 A1
(43) Pub. Date: Sep. 8, 2022

(54) ENGINEERED BIOSYNTHETIC PATHWAYS FOR PRODUCTION OF 3-AMINO-4-HYDROXYBENZOIC ACID BY FERMENTATION

(71) Applicant: Zymergen Inc., Emeryville, CA (US)

(72) Inventors: Cara Ann Tracewell, Walnut Creek, CA (US); Alexander Glennon Shearer, San Francisco, CA (US)

(73) Assignee: Zymergen Inc., Emeryville, CA (US)

(21) Appl. No.: 17/632,497

(22) PCT Filed: Aug. 10, 2020
(86) PCT No.: PCT/US20/70393

§ 371 Date: Feb. 2, 2022

Related U.S. Application Data
(60) Provisional application No. 62/885,790, filed on Aug. 12, 2019.

(51) Int. Cl. C12N 15/77; C12N 15/81; C12P 13/14

(52) U.S. Cl. CPC C12N 15/77; C12N 15/81; C12P 13/14; C12N 2510/02

(57) ABSTRACT

The present disclosure describes the engineering of microbial cells for fermentative production of 3-amino-4-hydroxybenzoic acid and provides novel engineered microbial cells and cultures, as well as related 3-amino-4-hydroxybenzoic acid production methods. Embodiments 1: An engineered microbial cell that produces 3-amino-4-hydroxybenzoic acid, wherein the engineered microbial cell expresses: (a) a non-native 2-amino-4,5-dihydroxy-6-oxo-7-(phosphooxy) heptanoate synthase; and (b) a non-native 3-amino-4-benzoic acid synthase.

### CROSS-REFERENCE TO RELATED APPLICATIONS

This application claims the benefit of U.S. provisional application No. 62/885,790, filed Aug. 12, 2019, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with Government support under Agreement No. HR0011-15-9-0014, awarded by DARPA. The Government has certain rights in the invention.

### INCORPORATION BY REFERENCE OF THE SEQUENCE LISTING

This application includes a sequence listing which has been submitted electronically in ASCII format and is hereby incorporated by reference in its entirety. This ASCII copy, created on Aug. 6, 2020, is named ZMGNP029WO_SeqList_ST25.txt. and is 41,058 bytes in size.

### FIELD OF THE DISCLOSURE

The present disclosure relates generally to the area of engineering microbes for production of 3-amino-4-hydroxybenzoic acid by fermentation.

### BACKGROUND

3-Amino-4-hydroxybenzoic acid is a precursor to antibiotics [1, 2] and other potential pharmaceuticals [3] and a monomer useful for sophisticated polymer materials, such as metal-organic framework materials capable of binding toxic molecules.

3-Amino-4-hydroxybenzoic acid is produced from dihydroxyacetone phosphate (DHAP) and aspartate semialdehyde by two enzymes, GriC and GriD [4, 5]. 3-amino-4-hydroxybenzoic acid has been produced in recombinant Corynebacteria glutamicum from sweet sorghum juice [6].

### SUMMARY

The disclosure provides engineered microbial cells, cultures of the microbial cells, and methods for the production of 3-amino-4-hydroxybenzoic acid, including the following:

Embodiments 1: An engineered microbial cell that produces 3-amino-4-hydroxybenzoic acid, wherein the engineered microbial cell expresses: (a) a non-native 2-amino-4,5-dihydroxy-6-oxo-7-(phosphooxy) heptanoate synthase; and (b) a non-native 3-amino-4-benzoic acid synthase.

Embodiment 2: The engineered microbial cell of embodiment 1, that includes increased activity of at least one or more upstream pathway enzyme(s) leading to: (a) L-aspartate semi-aldehyde; and/or (b) dihydroxyacetone phosphate (DHAP), said increased activity being increased relative to a control cell.

Embodiment 3: The engineered microbial cell of embodiment 2, wherein the engineered microbial cell includes increased activity of at least one or more upstream pathway enzyme(s) leading to L-aspartate semi-aldehyde.

Embodiment 4: The engineered microbial cell of embodiment 3, wherein the one or more upstream pathway enzyme(s) are selected from the group consisting of aspartate semi-aldehyde dehydrogenase, aspartokinase, aspartate aminotransferase, pyruvate carboxylase, phosphoenolpyruvate (PEP) carboxylase, PEP synthase, malate dehydrogenase, glutamate dehydrogenase, glutamate synthase, and glutamine synthetase.

Embodiment 5: The engineered microbial cell of embodiment 2, wherein the engineered microbial cell includes increased activity of at least one or more upstream pathway enzyme(s) leading to DHAP.

Embodiment 6: The engineered microbial cell of embodiment 5, wherein the one or more upstream pathway enzyme(s) comprise aldolase.

Embodiment 7: The engineered microbial cell of any one of embodiments 2-6, wherein the activity of the one or more upstream pathway enzyme(s) is increased by expressing an enzyme variant that has increased cytosolic localization, relative to that of the native enzyme.

Embodiment 8: The engineered microbial cell of embodiment 7, wherein the enzyme variant has a C-terminal truncation relative to the native enzyme.

Embodiment 9: The engineered microbial cell of embodiment 7 or embodiment 8, wherein the enzyme variant includes a variant of an enzyme selected from the group consisting of aspartate aminotransferase, pyruvate carboxylase, phosphoenolpyruvate (PEP) carboxylase, PEP synthase, malate dehydrogenase, and combinations thereof.

Embodiment 10: The engineered microbial cell of any one of embodiments 2-9, wherein the activity of the one or more upstream pathway enzyme(s) is increased by expressing one or more feedback-deregulated enzyme(s).

Embodiment 11: The engineered microbial cell of embodiment 10, where the one or more feedback-deregulated enzyme(s) are selected from the group consisting of a feedback-deregulated aspartate kinase, a feedback-deregulated aspartate semi-aldehyde dehydrogenase, and a feedback-deregulated pyruvate carboxylase.

Embodiment 12: The engineered microbial cell of embodiment 11, where the one or more feedback-deregulated enzyme(s) are selected from the group consisting of: (a) a feedback-deregulated Corynebacterium glutamicum ATCC 13032 aspartate kinase (UniProt ID P26512) including the amino acid substitution Q298G; (b) a feedback-deregulated aspartate-semialdehyde dehydrogenase (EC 1.2.1.11) including the amino acid substitutions D66G, S202F, R234H, D272E, and K285E; and (c) a feedback-deregulated pyruvate carboxylase (EC 6.4.1.1) including the amino acid substitution P458S.

Embodiment 13: The engineered microbial cell of embodiment 12, wherein the one or more feedback-deregulated enzyme(s) comprise a feedback-deregulated Corynebacterium glutamicum ATCC 13032 aspartate kinase (UniProt ID P26512) including the amino acid substitution Q298G.

Embodiment 14: The engineered microbial cell of any one of embodiments 1-13, wherein the engineered microbial cell includes reduced activity of one or more protein(s) that reduce the concentration of one or more upstream pathway precursor(s), said reduced activity being reduced relative to a control cell.

Embodiment 15: The engineered microbial cell of embodiment 14, wherein the one or more upstream precursor(s) comprise L-aspartate semi-aldehyde and/or dihydroxyacetone phosphate (DHAP).

Embodiment 16: The engineered microbial cell of embodiment 15, wherein the one or more upstream precursor(s) comprise L-aspartate semi-aldehyde.

Embodiment 17: The engineered microbial cell of embodiment 16, wherein the one or more protein(s) that reduce the concentration of L-aspartate semi-aldehyde are selected from the group consisting of homoserine dehydrogenase, 4-hydroxy-tetrahydrodipicolinate synthase, and phosphoenolpyruvate (PEP) carboxykinase.

Embodiment 18: The engineered microbial cell of embodiment 15, wherein the one or more upstream precursor(s) comprise DHAP.

Embodiment 19: The engineered microbial cell of embodiment 18, wherein the one or more protein(s) that reduce the concentration of DHAP are selected from the group consisting of glycerol-3-phosphate dehydrogenase, Saccharomyces cerevisiae FPS1 and its orthologs, triose phosphate isomerase, glycerol-3-phosphate/dihydroxyacetone phosphate acyltransferase, and pyruvate dehydrogenase.

Embodiment 20: The engineered microbial cell of any one of embodiments 14-19, wherein the reduced activity is achieved by one or more means selected from the group consisting of gene deletion, gene disruption, altering regulation of a gene, replacing a native promoter with a less active promoter; and expression of a protein variant having reduces activity.

Embodiment 21: The engineered microbial cell of any one of embodiments 1-20, wherein the engineered microbial cell includes increased activity of one or more enzyme(s) that increase the supply of the reduced form of nicotinamide adenine dinucleotide phosphate (NADPH), said increased activity being increased relative to a control cell.

Embodiment 22: The engineered microbial cell of embodiment 21, wherein the one or more enzyme(s) that increase the supply of the reduced form of NADPH are selected from the group consisting of pentose phosphate pathway enzymes, NADP+-dependent glyceraldehyde 3-phosphate dehydrogenase (GAPDH), and NADP+-dependent glutamate dehydrogenase.

Embodiment 23: The engineered microbial cell of any one of embodiments 1-22, wherein the engineered microbial cell includes altered cofactor specificity of one or more upstream pathway enzyme(s) from the reduced form of nicotinamide adenine dinucleotide phosphate (NADPH) to the reduced from of nicotinamide adenine dinucleotide (NADH).

Embodiment 24: The engineered microbial cell of embodiment 23, wherein the one or more upstream pathway enzyme(s) whose cofactor specificity is altered comprise aspartate semi-aldehyde dehydrogenase.

Embodiment 25: An engineered microbial cell that produces 3-amino-4-hydroxybenzoic acid, wherein the engineered microbial cell includes means for expressing: (a) a non-native 2-amino-4,5-dihydroxy-6-oxo-7-(phosphooxy) heptanoate synthase; and (b) a non-native 3-amino-4-benzoic acid synthase.

Embodiment 26: An engineered microbial cell that includes means for increasing the activity of at least one or more upstream pathway enzymes leading to: (a) L-aspartate semi-aldehyde; and/or (b) dihydroxyacetone phosphate (DHAP), said increased activity being increased relative to a control cell.

Embodiment 27: The engineered microbial cell of embodiment 26, wherein the engineered microbial cell includes means for increasing the activity of at least one or more upstream pathway enzymes leading to L-aspartate semi-aldehyde.

Embodiment 28: The engineered microbial cell of embodiment 27, wherein the one or more upstream pathway enzyme(s) are selected from the group consisting of aspartate semi-aldehyde dehydrogenase, apartokinase, aspartate aminotransferase, pyruvate carboxylase, phosphoenolpyruvate (PEP) carboxylase, PEP synthase, malate dehydrogenase, glutamate dehydrogenase, glutamate synthase, and glutamine synthetase.

Embodiment 29: The engineered microbial cell of embodiment 26, wherein the engineered microbial cell includes means for increasing that activity of at least one or more upstream pathway enzymes leading to DHAP.

Embodiment 30: The engineered microbial cell of embodiment 29, wherein the one or more upstream pathway enzyme(s) comprise aldolase.

Embodiment 31: The engineered microbial cell of any one of embodiments 26-30, wherein the engineered microbial cell includes means for expressing an enzyme variant that has increased cytosolic localization, relative to that of the native enzyme.

Embodiment 32: The engineered microbial cell of embodiment 31, wherein the enzyme variant has a C-terminal truncation relative to the native enzyme.

Embodiment 33: The engineered microbial cell of embodiment 31 or embodiment 32, wherein the enzyme variant includes a variant of an enzyme selected from the group consisting of aspartate aminotransferase, pyruvate carboxylase, phosphoenolpyruvate (PEP) carboxylase, PEP synthase, malate dehydrogenase, and combinations thereof.

Embodiment 34: The engineered microbial cell of any one of embodiments 26-33, wherein the engineered microbial cell includes means for expressing one or more feedback-deregulated enzyme(s).

Embodiment 35: The engineered microbial cell of embodiment 34, where the one or more feedback-deregulated enzyme (s) are selected from the group consisting of a feedback-deregulated aspartate kinase, a feedback-deregulated aspartate-semialdehyde dehydrogenase, and a feedback-deregulated pyruvate carboxylase.

Embodiment 36: The engineered microbial cell of embodiment 35, where the one or more feedback-deregulated enzyme(s) are selected from the group consisting of: (a) a feedback-deregulated Corynebacterium glutamicum ATCC 13032 aspartate kinase (UniProt ID P26512) including the amino acid substitution Q298G; (b) a feedback-deregulated aspartate-semialdehyde dehydrogenase (EC 1.2.1.11) including the amino acid substitutions D66G, S202F, R234H, D272E, and K285E; and (c) a feedback-deregulated pyruvate carboxylase (EC 6.4.1.1) including the amino acid substitution P458S.

Embodiment 37: The engineered microbial cell of embodiment 36, wherein the one or more feedback-deregulated enzyme(s) comprise a feedback-deregulated Corynebacterium glutamicum ATCC 13032 aspartate kinase (UniProt ID P26512) including the amino acid substitution Q298G.

Embodiment 38: The engineered microbial cell of any one of embodiments 25-37, wherein the engineered microbial cell includes means for reducing the activity of one or more protein(s) that reduce the concentration of one or more upstream pathway precursor(s), said reduced activity being reduced relative to a control cell.

Embodiment 39: The engineered microbial cell of embodiment 38, wherein the one or more upstream precursor(s) are L-aspartate semi-aldehyde and/or dihydroxyacetone phosphate (DHAP).

Embodiment 40: The engineered microbial cell of embodiment 39, wherein the one or more upstream precursor(s) comprise L-aspartate semi-aldehyde.

Embodiment 41: The engineered microbial cell of embodiment 40, wherein the one or more protein(s) that reduce the concentration of L-aspartate semi-aldehyde are selected from the group consisting of homoserine dehydrogenase, 4-hydroxy-tetrahydrodipicolinate synthase, and phosphoenolpyruvate (PEP) carboxykinase.

Embodiment 42: The engineered microbial cell of embodiment 39, wherein the one or more upstream precursor(s) comprise DHAP.

Embodiment 43: The engineered microbial cell of embodiment 42, wherein the one or more protein(s) that reduce the concentration of DHAP are selected from the group consisting of glycerol-3-phosphate dehydrogenase, Saccharomyces cerevisiae FPS1 and its orthologs, triose phosphate isomerase, glycerol-3-phosphate/dihydroxyacetone phosphate acyltransferase, and pyruvate dehydrogenase.

Embodiment 44: The engineered microbial cell of any one of embodiments 25-43, wherein the engineered microbial cell includes means for increasing the activity of one or more enzyme(s) that increase the supply of the reduced form of nicotinamide adenine dinucleotide phosphate (NADPH), said increased activity being increased relative to a control cell.

Embodiment 45: The engineered microbial cell of embodiment 44, wherein the one or more enzyme(s) that increase the supply of the reduced form of NADPH are selected from the group consisting of pentose phosphate pathway enzymes, NADP+-dependent glyceraldehyde 3-phosphate dehydrogenase (GAPDH), and NADP+-dependent glutamate dehydrogenase.

Embodiment 46: The engineered microbial cell of any one of embodiments 25-45, wherein the engineered microbial cell includes means for altering the cofactor specificity of one or more upstream pathway enzyme(s) from the reduced form of nicotinamide adenine dinucleotide phosphate (NADPH) to the reduced from of nicotinamide adenine dinucleotide (NADH).

Embodiment 47: The engineered microbial cell of embodiment 46, wherein the one or more upstream pathway enzyme(s) whose cofactor specificity is altered comprise aspartate semi-aldehyde dehydrogenase.

Embodiment 48: The engineered microbial cell of any one of embodiments 1-47, wherein: (a) the non-native 2-amino-4,5-dihydroxy-6-oxo-7-(phosphooxy) heptanoate synthase has at least 70% amino acid sequence identity with a Streptomyces sp. Root63 2-amino-4,5-dihydroxy-6-oxo-7-(phosphooxy) heptanoate synthase including SEQ ID NO:1; and (b) the non-native 3-amino-4-benzoic acid synthase has at least 70% amino acid sequence identity with a Saccharothrix espanaensis ATCC 51144 3-amino-4-benzoic acid synthase including SEQ ID NO:2.

Embodiment 49: The engineered microbial cell of embodiment 48, wherein: (a) the non-native 2-amino-4,5-dihydroxy-6-oxo-7-(phosphooxy) heptanoate synthase includes SEQ ID NO:1; and (b) the non-native 3-amino-4-benzoic acid synthase includes SEQ ID NO:2.

Embodiment 50: The engineered microbial cell of embodiment 48 or embodiment 49, wherein the engineered microbial cell is a bacterial cell.

Embodiment 51: The engineered microbial cell of embodiment 50, wherein the bacterial cell is a cell of the genus Corynebacteria.

Embodiment 52: The engineered microbial cell of embodiment 51, wherein the bacterial cell is a cell of the species glutamicum.

Embodiment 53: The engineered microbial cell of embodiment 48 or embodiment 49, wherein the engineered microbial cell includes a yeast cell.

Embodiment 54: The engineered microbial cell of embodiment 53, wherein the yeast cell is a cell of the genus Saccharomyces.

Embodiment 55: The engineered microbial cell of embodiment 54, wherein the yeast cell is a cell of the species cerevisiae.

Embodiment 56: The engineered microbial cell of embodiment 55, wherein: (a) the non-native 2-amino-4,5-dihydroxy-6-oxo-7-(phosphooxy) heptanoate synthase has at least 70% amino acid sequence identity with a Streptomyces thermoautotrophicus 2-amino-4,5-dihydroxy-6-oxo-7-(phosphooxy) heptanoate synthase including SEQ ID NO:5; and (b) the non-native 3-amino-4-benzoic acid synthase has at least 70% amino acid sequence identity with a Streptomyces griseus 3-amino-4-benzoic acid synthase including SEQ ID NO:4.

Embodiment 57: The engineered microbial cell of embodiment 56, wherein: (a) the non-native 2-amino-4,5-dihydroxy-6-oxo-7-(phosphooxy) heptanoate synthase includes SEQ ID NO:5; and (b) the non-native 3-amino-4-benzoic acid synthase includes SEQ ID NO:4.

Embodiment 58: The engineered microbial cell of any one of embodiments 1-52, wherein, when cultured, the engineered microbial cell produces 3-amino-4-hydroxybenzoic acid at a level of at least 20 μg/L of culture medium.

Embodiment 59: The engineered microbial cell of embodiment 58, wherein, when cultured, the engineered microbial cell produces 3-amino-4-hydroxybenzoic acid at a level of at least 4 mg/L of culture medium.

Embodiment 60: A culture of engineered microbial cells according to any one of embodiments 1-59.

Embodiment 61: The culture of embodiment 60, wherein the substrate includes a carbon source and a nitrogen source selected from the group consisting of urea, an ammonium salt, ammonia, and any combination thereof.

Embodiment 62: The culture of embodiment 60 or embodiment 61, wherein the engineered microbial cells are present in a concentration such that the culture has an optical density at 600 nm of 10-500.

Embodiment 63: The culture of any one of embodiments 60-62, wherein the culture includes 3-amino-4-hydroxybenzoic acid.

Embodiment 64: The culture of embodiment 63, wherein the culture includes 3-amino-4-hydroxybenzoic acid at a level of at least 20 μg/L of culture medium.

Embodiment 65: The culture of embodiment 64, wherein the culture includes 3-amino-4-hydroxybenzoic acid at a level of at least 4 mg/L of culture medium.

Embodiment 66: A method of culturing engineered microbial cells according to any one of embodiments 1-59, the method including culturing the cells under conditions suitable for producing 3-amino-4-hydroxybenzoic acid.

Embodiment 67: The method of embodiment 66, wherein the method includes fed-batch culture, with an initial glucose level in the range of 1-100 g/L, followed controlled sugar feeding.

Embodiment 68: The method of embodiment 66 or embodiment 67, wherein the fermentation substrate includes glucose and a nitrogen source selected from the group consisting of urea, an ammonium salt, ammonia, and any combination thereof.

Embodiment 69: The method of any one of embodiments 66-68, wherein the culture is pH-controlled during culturing.

Embodiment 70: The method of any one of embodiments 66-69, wherein the culture is aerated during culturing.

Embodiment 71: The method of any one of embodiments 66-70, wherein the engineered microbial cells produce 3-amino-4-hydroxybenzoic acid at a level of at least 20 μg/L of culture medium.

Embodiment 72: The method of embodiment 71, wherein the engineered microbial cells produce 3-amino-4-hydroxybenzoic acid at a level of at least 4 mg/L of culture medium.

Embodiment 73: The method of any one of embodiments 66-71, wherein the method additionally includes recovering 3-amino-4-hydroxybenzoic acid from the culture.

### BRIEF DESCRIPTION OF THE DRAWINGS

FIG. 1: Biosynthetic pathway for 3-amino-4-hydroxybenzoic acid.

FIG. 2: 3-Amino-4-hydroxybenzoic acid titers measured in the extracellular broth following fermentation by first-round engineered host Corynebacteria glutamicum.

FIG. 3: 3-Amino-4-hydroxybenzoic acid titers measured in the extracellular broth following fermentation by first-round engineered host Saccharomyces cerevisiae.

FIG. 4: 3-Amino-4-hydroxybenzoic acid titers measured in the extracellular broth following fermentation by the third-round engineered host C. glutamicum.

FIG. 5: Protein sequence similarity tree comparing GriI homologs of 2-amino-4,5-dihydroxy-6-oxo-7-(phosphooxy)heptanoate synthase tested in Saccharomyces cerevisiae for an improvement round of genetic engineering (see FIG. 11 for results).

FIG. 6: Protein sequence similarity tree comparing GriH homologs, 3-amino-4-hydroxybenzoic acid synthase tested in Saccharomyces cerevisiae for an improvement round of genetic engineering (see FIG. 11 for results).

FIG. 7: 3-Amino-4-hydroxybenzoic acid titers measured in the extracellular broth following fermentation by first-round engineered host Yarrowia lipolytica.

FIG. 8: 3-Amino-4-hydroxybenzoic acid titers measured in the extracellular broth following fermentation by first-round engineered host Bacillus subtillus.

FIG. 9: 3-Amino-4-hydroxybenzoic acid titers measured in the extracellular broth following fermentation by S. cerevisiae testing host evaluation designs.

FIG. 10: 3-Amino-4-hydroxybenzoic acid titers measured in the extracellular broth following fermentation by C. glutamicum testing host evaluation designs.

FIG. 11: 3-Amino-4-hydroxybenzoic acid titers measured in the extracellular broth following fermentation by S. cerevisiae testing improvement designs.

FIG. 12: Integration of Promoter-Gene-Terminator into Saccharomyces cerevisiae and Yarrowia lipolytica.

FIG. 13: Integration of Promoter-Gene-Terminator into Corynebacteria glutamicum and Bacillus subtilis.

### DETAILED DESCRIPTION

This disclosure describes a method for the production of the small molecule 3-amino-4-hydroxybenzoic acid via fermentation by a microbial host from simple carbon and nitrogen sources, such as glucose and urea, respectively. This aim was achieved via the introduction of a non-native metabolic pathway into a suitable microbial host for industrial fermentation of large-scale chemical products, such as Saccharomyces cerevisiae and Yarrowia lipolytica. The engineered metabolic pathway links the central metabolism of the host to the non-native pathway to enable the production of 3-amino-4-hydroxybenzoic acid. The simplest embodiment of this method is the expression of two non-native enzymes, 2-amino-4,5-dihydroxy-6-oxo-7-(phosphooxy) heptanoate synthase and 3-amino-4-benzoic acid synthase, in the microbial host. Over-expression of certain upstream pathway enzymes enabled titers of 3.3 mg/L 3-amino-4-hydroxybenzoic acid in Saccharomyces cerevisiae.

### Definitions

Terms used in the claims and specification are defined as set forth below unless otherwise specified.

The term “fermentation” is used herein to refer to a process whereby a microbial cell converts one or more substrate(s) into a desired product (such as 3-amino-4-hydroxybenzoic acid) by means of one or more biological conversion steps, without the need for any chemical conversion step.

The term “engineered” is used herein, with reference to a cell, to indicate that the cell contains at least one targeted genetic alteration introduced by man that distinguishes the engineered cell from the naturally occurring cell.

The term “native” is used herein to refer to a cellular component, such as a polynucleotide or polypeptide, that is naturally present in a particular cell. A native polynucleotide or polypeptide is endogenous to the cell.

When used with reference to a polynucleotide or polypeptide, the term “non-native” refers to a polynucleotide or polypeptide that is not naturally present in a particular cell.

When used with reference to the context in which a gene is expressed, the term “non-native” refers to a gene expressed in any context other than the genomic and cellular context in which it is naturally expressed. A gene expressed in a non-native manner may have the same nucleotide sequence as the corresponding gene in a host cell, but may be expressed from a vector or from an integration point in the genome that differs from the locus of the native gene.

The term “heterologous” is used herein to describe a polynucleotide or polypeptide introduced into a host cell. This term encompasses a polynucleotide or polypeptide, respectively, derived from a different organism, species, or strain than that of the host cell. In this case, the heterologous polynucleotide or polypeptide has a sequence that is different from any sequence(s) found in the same host cell. However, the term also encompasses a polynucleotide or polypeptide that has a sequence that is the same as a sequence found in the host cell, wherein the polynucleotide or polypeptide is present in a different context than the native sequence (e.g., a heterologous polynucleotide can be linked to a different promotor and inserted into a different genomic location than that of the native sequence). “Heterologous expression” thus encompasses expression of a sequence that is non-native to the host cell, as well as expression of a sequence that is native to the host cell in a non-native context.

As used with reference to polynucleotides or polypeptides, the term “wild-type” refers to any polynucleotide having a nucleotide sequence, or polypeptide having an amino acid, sequence present in a polynucleotide or polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized. The term “wild-type” is also used to denote naturally occurring cells.

A “control cell” is a cell that is otherwise identical to an engineered cell being tested, including being of the same genus and species as the engineered cell, but lacks the specific genetic modification(s) being tested in the engineered cell.

Enzymes are identified herein by the reactions they catalyze and, unless otherwise indicated, refer to any polypeptide capable of catalyzing the identified reaction. Unless otherwise indicated, enzymes may be derived from any organism and may have a native or mutated amino acid sequence. As is well known, enzymes may have multiple functions and/or multiple names, sometimes depending on the source organism from which they derive. The enzyme names used herein encompass orthologs, including enzymes that may have one or more additional functions or a different name.

The term “feedback-deregulated” is used herein with reference to an enzyme that is normally negatively regulated by a downstream product of the enzymatic pathway (i.e., feedback-inhibition) in a particular cell. In this context, a “feedback-deregulated” enzyme is a form of the enzyme that is less sensitive to feedback-inhibition than the enzyme native to the cell or a form of the enzyme that is native to the cell, but is naturally less sensitive to feedback inhibition than one or more other natural forms of the enzyme. A feedback-deregulated enzyme may be produced by introducing one or more mutations into a native enzyme. Alternatively, a feedback-deregulated enzyme may simply be a heterologous, native enzyme that, when introduced into a particular microbial cell, is not as sensitive to feedback-inhibition as the native, native enzyme. In some embodiments, the feedback-deregulated enzyme shows no feedback-inhibition in the microbial cell.

The term “3-amino-4-hydroxybenzoic acid” refers to a chemical compound of the formula C₇H₇NO₃ (CAS #1571-72-8).

The term “sequence identity,” in the context of two or more amino acid or nucleotide sequences, refers to two or more sequences that are the same or have a specified percentage of amino acid residues or nucleotides that are the same, when compared and aligned for maximum correspondence, as measured using a sequence comparison algorithm or by visual inspection.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST set to default parameters.

The term “titer,” as used herein, refers to the mass of a product (e.g., 3-amino-4-hydroxybenzoic acid) produced by a culture of microbial cells divided by the culture volume.

As used herein with respect to recovering 3-amino-4-hydroxybenzoic acid from a cell culture, “recovering” refers to separating the 3-amino-4-hydroxybenzoic acid from at least one other component of the cell culture medium.

### Engineering Microbes for 3-Amino-4-Hydroxybenzoic Acid Production

### 3-Amino-4-Hydroxybenzoic Acid Biosynthesis Pathway

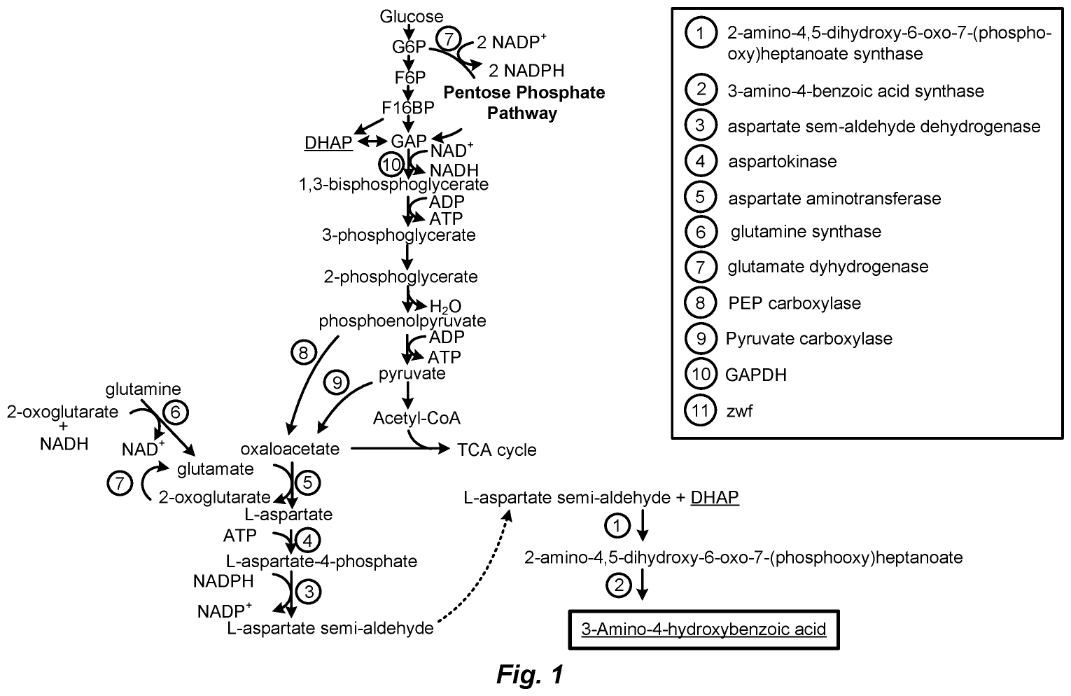

3-amino-4-hydroxybenzoic acid can be produced from L-aspartate semi-aldehyde and dihydroxyacetone phosphate (DHAP) in two enzymatic steps, requiring the enzyme 2-amino-4,5-dihydroxy-6-oxo-7-(phosphooxy) heptanoate synthase and the enzyme 3-amino-4-hydroxybenzoic acid synthase. The 3-amino-4-hydroxybenzoic acid biosynthesis pathway is shown in FIG. 1. Accordingly, a microbial host that can produce the precursors L-aspartate semi-aldehyde and DHAP can be engineered to produce 3-amino-4-hydroxybenzoic acid by expressing forms of a 2-amino-4,5-dihydroxy-6-oxo-7-(phosphooxy) heptanoate synthase and a 3-amino-4-hydroxybenzoic acid synthase that are active in the microbial host.

### Engineering for Microbial 3-Amino-4-Hydroxybenzoic Acid Production

Any 2-amino-4,5-dihydroxy-6-oxo-7-(phosphooxy) heptanoate synthase and 3-amino-4-hydroxybenzoic acid synthase that is active in the microbial cell being engineered may be introduced into the cell, typically by introducing and expressing the gene(s) encoding the enzyme(s)s using standard genetic engineering techniques. Suitable 2-amino-4,5-dihydroxy-6-oxo-7-(phosphooxy) heptanoate synthases and 3-amino-4-hydroxybenzoic acid beta-synthases may be derived from any source, including plant, archaeal, fungal, gram-positive bacterial, and gram-negative bacterial sources.

One or more copies of any of these genes can be introduced into a selected microbial host cell. If more than one copy of a gene is introduced, the copies can have the same or different nucleotide sequences. In some embodiments, one or both (or all) of the heterologous gene(s) is/are expressed from a strong, constitutive promoter. In some embodiments, the heterologous gene(s) is/are expressed from an inducible promoter. The heterologous gene(s) can optionally be codon-optimized to enhance expression in the selected microbial host cell. The codon-optimization tables used in the Examples are as follows: Bacillus subtilis Kazusa codon table: www.kazusa.or.jp/codon/cgi-bin/showcodon.cgi?species=1423&aa=1&style=N; Yarrowia lipolytica Kazusa codon table: www.kazusa.or.jp/codon/cgi-bin/showcodon.cgi?species=4952&aa=1&style=N; Corynebacteria glutamicum Kazusa codon table: www.kazusa.or.jp/codon/cgi-bin/showcodon.cgi?species=340322&aa=1&style=N; Saccharomyces cerevisiae Kazusa codon table: www.kazusa.or.jp/codon/cgi-bin/showcodon.cgi?species=4932&aa=1&style=N. Also used, was a modified, combined codon usage scheme for S. cereviae and C. glutamicum, which is reproduced below.

Modified Codon Usage Table for Sc and Cg

| Amino | | |
|---|---|---|
| Acid | Codon | Fraction |
| A | GCG | 0.22 |
| A | GCA | 0.29 |
| A | GCT | 0.24 |
| A | GCC | 0.25 |
| C | TGT | 0.36 |
| C | TGC | 0.64 |
| D | GAT | 0.56 |
| D | GAC | 0.44 |
| E | GAG | 0.44 |
| E | GAA | 0.56 |
| F | TTT | 0.37 |
| F | TTC | 0.63 |
| G | GGG | 0.08 |
| G | GGA | 0.19 |
| G | GGT | 0.3 |
| G | GGC | 0.43 |
| H | CAT | 0.32 |
| H | CAC | 0.68 |
| I | ATA | 0.03 |
| I | ATT | 0.38 |
| I | ATC | 0.59 |
| K | AAG | 0.6 |
| K | AAA | 0.4 |
| L | TTG | 0.29 |
| L | TTA | 0.05 |
| L | CTG | 0.29 |
| L | CTA | 0.06 |
| L | CTT | 0.17 |
| L | CTC | 0.14 |
| M | ATG | 1 |
| N | AAT | 0.33 |
| N | AAC | 0.67 |
| P | CCG | 0.22 |
| P | CCA | 0.35 |
| P | CCT | 0.23 |
| P | CCC | 0.2 |
| Q | CAG | 0.61 |
| Q | CAA | 0.39 |
| R | AGG | 0.11 |
| R | AGA | 0.12 |
| R | CGG | 0.09 |
| R | CGA | 0.17 |
| R | CGT | 0.34 |
| R | CGC | 0.18 |
| S | AGT | 0.08 |
| S | AGC | 0.16 |
| S | TCG | 0.12 |
| S | TCA | 0.13 |
| S | TCT | 0.17 |
| S | TCC | 0.34 |
| T | ACG | 0.14 |
| T | ACA | 0.12 |
| T | ACT | 0.2 |
| T | ACC | 0.53 |
| V | GTG | 0.36 |
| V | GTA | 0.1 |
| V | GTT | 0.26 |
| V | GTC | 0.28 |
| W | TGG | 1 |
| Y | TAT | 0.34 |
| Y | TAC | 0.66 |

In Corynebacteria glutamicum, for example, an about 4.6 mg/L titer of 3-amino-4-hydroxybenzoic acid was achieved in the best-performing strain tested by expressing a 2-amino-4,5-dihydroxy-6-oxo-7-(phosphooxy)heptanoate synthase from Streptomyces sp. Root63 (UniProt ID A0A0Q8F363) and 3-amino-4-benzoic acid synthase from Saccharothrix espanaensis ATCC 51144 (UniProt ID K0JXI9) (see Example 1, FIG. 2, and Table 1 below).

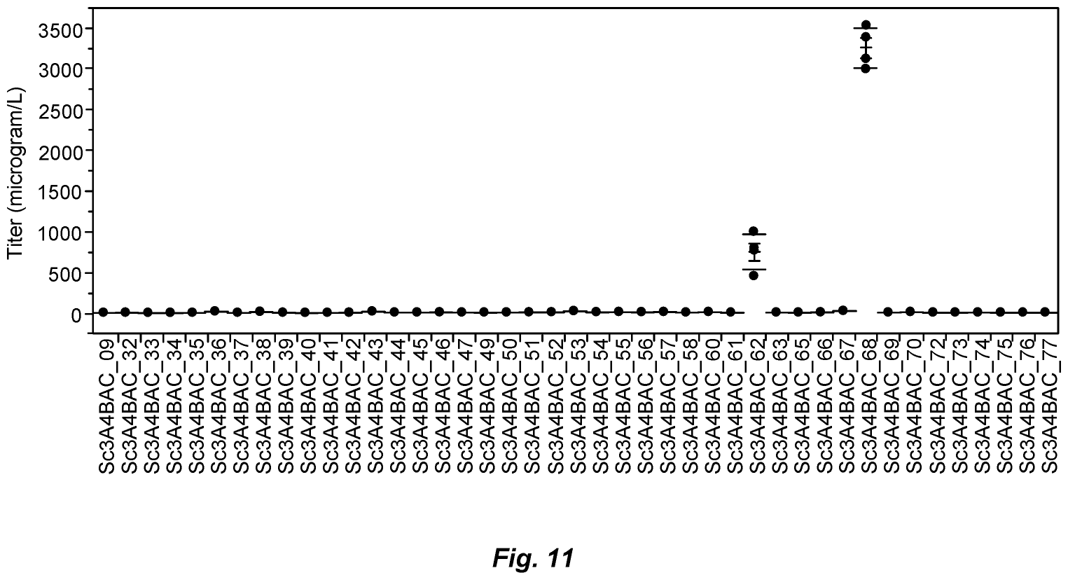

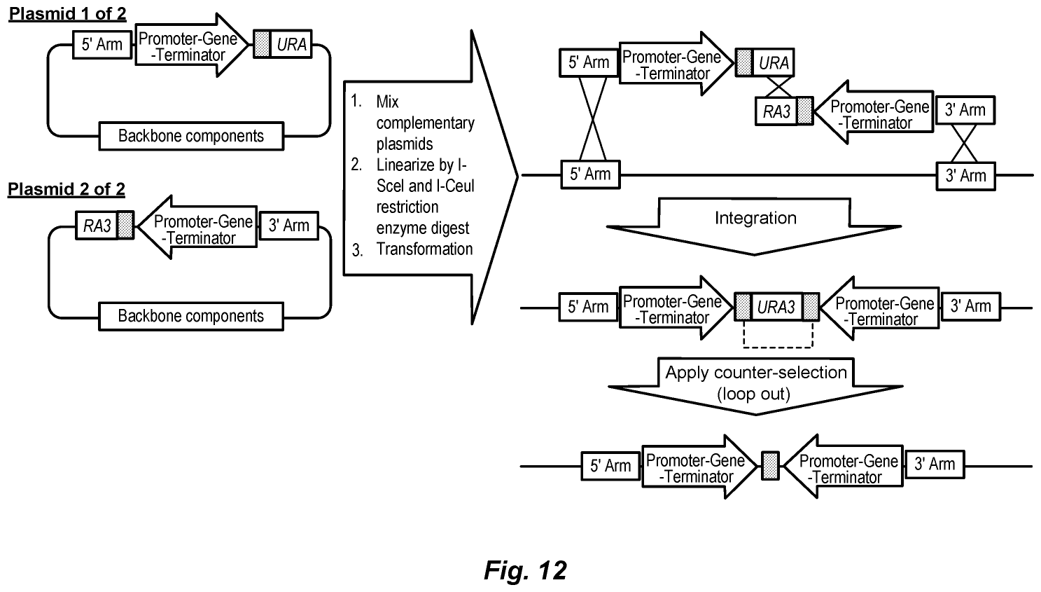

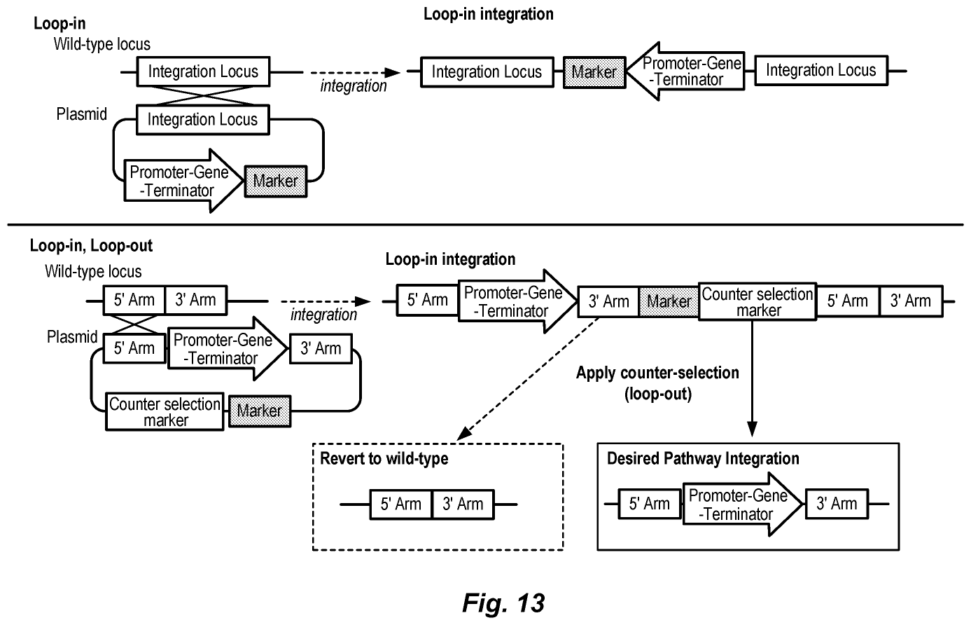

In Saccharomyces cerevisiae, the best-performing strains tested contained the same two enzymes as the best-performing C. glutamicum strain. For example, an about 753 μg/L titer of 3-amino-4-hydroxybenzoic acid was achieved by expressing these two enzymes and a 2-amino-4,5-dihydroxy-6-oxo-7-(phosphooxy)heptanoate synthase (NCBI-OFX87022) from Bacteroidetes bacterium GWE2_32_14 and 3-amino-4-benzoic acid synthase (A0JC76) from Streptomyces griseus. An about 3.3 mg/L titer of 3-amino-4-hydroxybenzoic acid was achieved by expressing the 2-amino-4,5-dihydroxy-6-oxo-7-(phosphooxy)heptanoate synthase from Streptomyces sp. Root63 and 3-amino-4-benzoic acid synthase from Saccharothrix espanaensis ATCC 51144, together with a 2-amino-4,5-dihydroxy-6-oxo-7-(phosphooxy)heptanoate synthase (A0A132MRF8) from Streptomyces thermoautotrophicus and 3-amino-4-hydroxybenzoic acid synthase (A0JC76) from Streptomyces griseus. (See Example 3, FIG. 11, and Table 8, below).

### Increasing the Activity of Upstream Enzymes

One approach to increasing 3-amino-4-hydroxybenzoic acid production in a microbial cell that is capable of such production is to increase the activity of one or more upstream enzymes leading to the precursors L-aspartate semi-aldehyde and/or DHAP. Upstream pathway enzymes include all enzymes involved in the conversions from a feedstock all the way to a L-aspartate semi-aldehyde and/or DHAP. Illustrative enzymes, for this purpose, include, but are not limited to, those shown in FIG. 1 in the pathways leading to these precursors. Suitable upstream pathway genes encoding these enzymes may be derived from any available source, including, for example, those disclosed herein.

In some embodiments, the activity of one or more upstream pathway enzymes is increased by modulating the expression or activity of the native enzyme(s). For example, native regulators of the expression or activity of such enzymes can be exploited to increase the activity of suitable enzymes.

Alternatively, or in addition, one or more promoters can be substituted for native promoters. In certain embodiments, the replacement promoter is stronger than the native promoter and/or is a constitutive promoter.

In some embodiments, the activity of one or more upstream pathway enzymes is supplemented by introducing one or more of the corresponding genes into the engineered microbial host cell. An introduced upstream pathway gene may be from an organism other than that of the host cell or may simply be an additional copy of a native gene. In some embodiments, one or more such genes are introduced into a microbial host cell capable of 3-amino-4-hydroxybenzoic acid production and expressed from a strong constitutive promoter and/or can optionally be codon-optimized to enhance expression in the selected microbial host cell.

Expressing Cytosolic Variants of Enzymes that are Normally Expressed or Trafficked Elsewhere in the Cell

In some embodiment the “effective activity” activity of an enzyme (e.g., an upstream pathway enzyme can be increased by expressing one or more enzyme variants that have increased cytosolic localization relative to that of the native enzyme. Increased “effective activity” refers to an enhancement in conversion of substrate to product by virtue of the enzyme localizing to a cellular compartment in which the substrate is primarily found (of being generated). Suitable variants may be naturally occurring enzyme variants or recombinantly produced variant. For example, Zelle et al. were able to improve malate production in Saccharomyces CEN.PK by expressing pyruvate carboxylase and a modified malate dehydrogenase, which is normally found in the peroxisome; in this case, truncation of the three C-terminal amino acids of malate dehydrogenase resulted in cytosolic expression of the enzyme [7]. Similar improvements in 3-amino-4-hydroxybenzoic acid production are expected for an analogous C-terminal truncation of one or more upstream pathway enzyme(s) leading to the precursors L-aspartate semi-aldehyde and/or DHAP, where the enzyme(s) predominantly localize to non-cytosolic cellular compartments. Illustrative enzymes for this purpose include those discussed in the Summary above.

In various embodiments, the engineering of a 3-amino-4-hydroxybenzoic acid-producing microbial cell to increase the activity of one or more upstream pathway enzymes increases the 3-amino-4-hydroxybenzoic acid titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 3-amino-4-hydroxybenzoic acid titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the 3-amino-4-hydroxybenzoic acid titer observed in a 3-amino-4-hydroxybenzoic acid-producing microbial cell that lacks any increase in activity of upstream pathway enzymes. This reference cell may have one or more other genetic alterations aimed at increasing 3-amino-4-hydroxybenzoic acid production.

In various embodiments, the 3-amino-4-hydroxybenzoic acid titers achieved by increasing the activity of one or more upstream pathway enzymes are at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 mg/L, 200 μg/L to 40 gm/L, 300 μg/L to 30 gm/L, 500 μg/L to 25 mg/L, 1 mg/L to 20 mg/L, or any range bounded by any of the values listed above.

### Feedback-Deregulated Enzymes

Another approach to increasing 3-amino-4-hydroxybenzoic acid production in a microbial cell engineered for enhanced 3-amino-4-hydroxybenzoic acid production is to introduce feedback-deregulated forms of one or more enzymes that are normally subject to feedback regulation (e.g., those discussed above in the Summary). A feedback-deregulated form can be a heterologous, native enzyme that is less sensitive to feedback inhibition than the native enzyme in the particular microbial host cell. Alternatively, a feedback-deregulated form can be a variant of a native or heterologous enzyme that has one or more mutations or truncations rendering it less sensitive to feedback inhibition than the corresponding native enzyme.

In some embodiments, the feedback-deregulated enzyme need not be “introduced,” in the traditional sense. Rather, the microbial host cell selected for engineering can be one that has a native enzyme that is naturally insensitive to feedback inhibition.

In various embodiments, the engineering of a 3-amino-4-hydroxybenzoic acid-producing microbial cell to include one or more feedback-regulated enzymes increases the 3-amino-4-hydroxybenzoic acid titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 3-amino-4-hydroxybenzoic acid titer is in the range of 10-fold to 1000-fold, 20-fold to 500-fold, 50-fold to 400-fold, 10-fold to 300-fold, or any range bounded by any of the values listed above. These increases are determined relative to the 3-amino-4-hydroxybenzoic acid titer observed in a 3-amino-4-hydroxybenzoic acid-producing microbial cell that does not include genetic alterations to reduce feedback regulation. This reference cell may (but need not) have other genetic alterations aimed at increasing 3-amino-4-hydroxybenzoic acid production, i.e., the cell may have increased activity of an upstream pathway enzyme.

In various embodiments, the 3-amino-4-hydroxybenzoic acid titers achieved by reducing feedback deregulation are at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 mg/L, 200 μg/L to 40 gm/L, 300 μg/L to 30 gm/L, 500 μg/L to 25 mg/L, 1 mg/L to 20 mg/L, or any range bounded by any of the values listed above.

### Reduction of Consumption of 3-Amino-4-Hydroxybenzoic Acid and/or its Precursors

Another approach to increasing 3-amino-4-hydroxybenzoic acid production in a microbial cell that is capable of such production is to decrease the activity of one or more enzymes that consume one or more 3-amino-4-hydroxybenzoic acid pathway precursors (e.g., precursors L-aspartate semi-aldehyde and/or DHAP) or that consume 3-amino-4-hydroxybenzoic acid itself (see those discussed above in the Summary). In some embodiments, the activity of one or more such enzymes is reduced by modulating the expression or activity of the native enzyme(s). The activity of such enzymes can be decreased, for example, by substituting the native promoter of the corresponding gene(s) with a less active or inactive promoter or by deleting the corresponding gene(s).

In various embodiments, the engineering of a 3-amino-4-hydroxybenzoic acid-producing microbial cell to reduce precursor consumption by one or more side pathways increases the 3-amino-4-hydroxybenzoic acid titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 3-amino-4-hydroxybenzoic acid titer is in the range of 10-fold to 1000-fold, 20-fold to 500-fold, 50-fold to 400-fold, 10-fold to 300-fold, or any range bounded by any of the values listed above. These increases are determined relative to the 3-amino-4-hydroxybenzoic acid titer observed in a 3-amino-4-hydroxybenzoic acid-producing microbial cell that does not include genetic alterations to reduce precursor consumption. This reference cell may (but need not) have other genetic alterations aimed at increasing 3-amino-4-hydroxybenzoic acid production, i.e., the cell may have increased activity of an upstream pathway enzyme.

In various embodiments, the 3-amino-4-hydroxybenzoic acid titers achieved by reducing precursor consumption are at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 mg/L, 200 μg/L to 40 gm/L, 300 μg/L to 30 gm/L, 500 μg/L to 25 mg/L, 1 mg/L to 20 mg/L, or any range bounded by any of the values listed above.

Any of the approaches for increasing 3-amino-4-hydroxybenzoic acid production described above can be combined, in any combination, to achieve even higher 3-amino-4-hydroxybenzoic acid production levels.

### Increasing the NADPH Supply

Another approach to increasing 3-amino-4-hydroxybenzoic acid production in a microbial cell that is capable of such production is to increase the supply of the reduced form of nicotinamide adenine dinucleotide phosphate (NADPH), which provides the reducing equivalents for biosynthetic reactions. For example, the activity of one or more enzymes that increase the NADPH supply can be increased by means similar to those described above for upstream pathway enzymes, e.g., by modulating the expression or activity of the native enzyme(s), replacing the native promoter(s) with a stronger and/or constitutive promoter, and/or introducing one or more gene(s) encoding enzymes that increase the NADPH supply. Illustrative enzymes, for this purpose, include, but are not limited to, pentose phosphate pathway enzymes, NADP+-dependent glyceraldehyde 3-phosphate dehydrogenase (GAPDH), and NADP+-dependent glutamate dehydrogenase. Such enzymes may be derived from any available source, including, for example, any of those described herein with respect to other enzymes. Examples include the NADPH-dependent glyceraldehyde 3-phosphate dehydrogenase (GAPDH) encoded by gapC from Clostridium acetobutylicum, the NADPH-dependent GAPDH encoded by gapB from Bacillus subtilis, and the non-phosphorylating GAPDH encoded by gapN from Streptococcus mutans. The yield of 3-amino-4-hydroxybenzoic acid can also enhanced by altering the cofactor specificity of NADP+-dependent enzymes such as GAPDH and glutamate dehydrogenase, e.g., to use NADPH preferentially over NADH (as discussed below) and providing NADPH to pathway enzymes without the loss of CO₂.

In various embodiments, the engineering of a 3-amino-4-hydroxybenzoic acid-producing microbial cell to increase the activity of one or more of such enzymes increases the 3-amino-4-hydroxybenzoic acid titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 3-amino-4-hydroxybenzoic acid titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the 3-amino-4-hydroxybenzoic acid titer observed in a 3-amino-4-hydroxybenzoic acid-producing microbial cell that lacks any increase in activity of such enzymes. This reference cell may have one or more other genetic alterations aimed at increasing 3-amino-4-hydroxybenzoic acid production.

In various embodiments, the 3-amino-4-hydroxybenzoic acid titers achieved by increasing the activity of one or more enzymes that increase the NADPH supply are at least 10, 20, 30, 40, 50, 75, 100, 200, 300, 400, 500, 600, 700, 800, or 900 mg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 gm/L. In various embodiments, the titer is in the range of 10 mg/L to 150 gm/L, 20 mg/L to 140 gm/L, 50 mg/L to 130 gm/L, 100 mg/L to 120 gm/L, 500 mg/L to 110 gm/L or any range bounded by any of the values listed above.

Altering the Cofactor Specificity of Upstream Pathway Enzymes

Another approach to increasing 3-amino-4-hydroxybenzoic acid production in a microbial cell that is capable of such production is to alter the cofactor specificity of an upstream pathway enzyme that typically prefers the reduced form of nicotinamide adenine dinucleotide phosphate (NADPH) to the reduced from of nicotinamide adenine dinucleotide (NADH) (see those discussed above in the Summary), which provides the reducing equivalents for biosynthetic reactions. This can be achieved, for example, by expressing one or more variants of such enzymes that have the desired altered cofactor specificity. Examples of upstream pathway enzymes that rely on NADPH, and for which suitable variants are known, include aspartate semi-aldehyde dehydrogenase. Mining of natural NADH-utilizing dehydrogenases has yielded enzymes such as aspartate semi-aldehyde dehydrogenase from Tistrella mobilis that use NADH [11]. In addition, several examples of altering the cofactor specificity of enzymes to use NADH preferentially to NADPH are known [12-14]. The yield enhancement from altering the cofactor specificity of such enzymes arises from decreased pentose phosphate flux which produces NADPH but also results in CO₂ loss by 6-phosphogluconate dehydrogenase (gnd) [15].

In various embodiments, the engineering of a 3-amino-4-hydroxybenzoic acid-producing microbial cell to alter the cofactor specificity of one or more of such enzymes increases the 3-amino-4-hydroxybenzoic acid titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 3-amino-4-hydroxybenzoic acid titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the 3-amino-4-hydroxybenzoic acid titer observed in a 3-amino-4-hydroxybenzoic acid-producing microbial cell that lacks any increase in activity of such enzymes. This reference cell may have one or more other genetic alterations aimed at increasing 3-amino-4-hydroxybenzoic acid production.

In various embodiments, the 3-amino-4-hydroxybenzoic acid titers achieved by altering the cofactor specificity of one or more enzymes that typically rely on NADPH as a cofactor are at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 mg/L, 200 μg/L to 40 gm/L, 300 μg/L to 30 gm/L, 500 μg/L to 25 mg/L, 1 mg/L to 20 mg/L, or any range bounded by any of the values listed above.

Illustrative Amino Acid and Nucleotide Sequences

The following table identifies amino acid and nucleotide sequences used in Example 1. The corresponding sequences are shown in the Sequence Listing.

SEQ ID NO Cross-Reference Table

| | AA |
|---|---|
| | SEQ ID |
| Enzyme Description | NO: |
| 2-amino-4,5-dihydroxy-6-oxo-7-(phosphooxy)heptanoate synthase (A0A0Q8F363) from Streptomyces sp. | 1 |
| Root63 | |
| 3-amino-4-benzoic acid synthase (K0JXI9) from Saccharothrix espanaensis ATCC 51144 | 2 |
| 2-amino-4,5-dihydroxy-6-oxo-7-(phosphooxy)heptanoate synthase (NCBI-OFX87022) from Bacteroidetes | 3 |
| bacterium GWE2_32_14 | |
| 3-amino-4-benzoic acid synthase (A0JC76) from Streptomyces griseus | 4 |
| 2-amino-4,5-dihydroxy-6-oxo-7-(phosphooxy)heptanoate synthase (A0A132MRF8) from Streptomyces | 5 |
| thermoautotrophicus | |
| 3-amino-4-benzoic acid synthase (W5WBR4) from Kutzneria albida DSM 43870 | 6 |
| 2-amino-4,5-dihydroxy-6-oxo-7-(phosphooxy)heptanoate synthase (NCBI-CUB39904.1) from Bacillus | 7 |
| cereus | |
| 3-dehydroquinate synthase (A0A1T3V8D3) from Bacillus anthracis | 8 |
| 2-amino-4,5-dihydroxy-6-oxo-7-(phosphooxy)heptanoate synthase (A0JC77) from Streptomyces griseus | 9 |
| 3-amino-4-benzoic acid synthase (A0A0K2YDP9) from Rhodococcus sp. RD6.2 | 10 |
| 2-amino-4,5-dihydroxy-6-oxo-7-(phosphooxy) heptanoate synthase (A0A0M4DD67) from Streptomyces | 11 |
| pristinaespiralis | |
| 2-amino-4,5-dihydroxy-6-oxo-7-(phosphooxy) heptanoate (K0KAK8) synthase from Saccharothrix | 12 |
| espanaensis ATCC 51144 | |
| 3-amino-4-benzoic acid synthase (A0A0K6LJE3) from Bacillus cereus | 13 |
| Aspartokinase (P26512) from Corynebacterium glutamicum ATCC 13032 | 14 |

Microbial Host Cells

Any microbe that can be used to express introduced genes can be engineered for fermentative production of 3-amino-4-hydroxybenzoic acid as described above. In certain embodiments, the microbe is one that is naturally incapable of fermentative production of 3-amino-4-hydroxybenzoic acid. In some embodiments, the microbe is one that is readily cultured, such as, for example, a microbe known to be useful as a host cell in fermentative production of compounds of interest. Bacteria cells, including gram-positive or gram-negative bacteria can be engineered as described above. Examples include, in addition to C. glutamicum cells, Bacillus subtilus, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., P. alcaligenes, P. citrea, Lactobacillus spp. (such as L. lactis, L. plantarum), L. grayi, E. coli, E. faecium, E. gallinarum, E. casseliflavus, and/or E. faecalis cells.

There are numerous types of anaerobic cells that can be used as microbial host cells in the methods described herein. In some embodiments, the microbial cells are obligate anaerobic cells. Obligate anaerobes typically do not grow well, if at all, in conditions where oxygen is present. It is to be understood that a small amount of oxygen may be present, that is, there is some level of tolerance level that obligate anaerobes have for a low level of oxygen. Obligate anaerobes engineered as described above can be grown under substantially oxygen-free conditions, wherein the amount of oxygen present is not harmful to the growth, maintenance, and/or fermentation of the anaerobes.

Alternatively, the microbial host cells used in the methods described herein can be facultative anaerobic cells. Facultative anaerobes can generate cellular ATP by aerobic respiration (e.g., utilization of the TCA cycle) if oxygen is present. However, facultative anaerobes can also grow in the absence of oxygen. Facultative anaerobes engineered as described above can be grown under substantially oxygen-free conditions, wherein the amount of oxygen present is not harmful to the growth, maintenance, and/or fermentation of the anaerobes, or can be alternatively grown in the presence of greater amounts of oxygen.

In some embodiments, the microbial host cells used in the methods described herein are filamentous fungal cells. (See, e.g., Berka & Barnett, Biotechnology Advances, (1989), 7(2):127-154). Examples include Trichoderma longibrachiatum, T. viride, T. koningii, T. harzianum, Penicillium sp., Humicola insolens, H. lanuginose, H. grisea, Chrysosporium sp., C. lucknowense, Gliocladium sp., Aspergillus sp. (such as A. oryzae, A. niger, A. sojae, A. japonicus, A. nidulans, or A. awamori), Fusarium sp. (such as F. roseum, F. graminum F. cerealis, F. oxysporuim, or F. venenatum), Neurospora sp. (such as N. crassa or Hypocrea sp.), Mucor sp. (such as M. miehei), Rhizopus sp., and Emericella sp. cells. In particular embodiments, the fungal cell engineered as described above is A. nidulans, A. awamori, A. oryzae, A. aculeatus, A. niger, A. japonicus, T. reesei, T. viride, F. oxysporum, or F. solani. Illustrative plasmids or plasmid components for use with such hosts include those described in U.S. Patent Pub. No. 2011/0045563.

Yeasts can also be used as the microbial host cell in the methods described herein. Examples include: Saccharomyces sp., Schizosaccharomyces sp., Pichia sp., Hansenula polymorpha, Pichia stipites, Kluyveromyces marxianus, Kluyveromyces spp., Yarrowia lipolytica and Candida sp. In some embodiments, the Saccharomyces sp. is S. cerevisiae (See, e.g., Romanos et al., Yeast, (1992), 8(6):423-488). Illustrative plasmids or plasmid components for use with such hosts include those described in U.S. Pat. No. 7,659,097 and U.S. Patent Pub. No. 2011/0045563.

In some embodiments, the host cell can be an algal cell derived, e.g., from a green alga, red alga, a glaucophyte, a chlorarachniophyte, a euglenid, a chromista, or a dinoflagellate. (See, e.g., Saunders & Warmbrodt, “Gene Expression in Algae and Fungi, Including Yeast,” (1993), National Agricultural Library, Beltsville, Md.). Illustrative plasmids or plasmid components for use in algal cells include those described in U.S. Patent Pub. No. 2011/0045563.

In other embodiments, the host cell is a cyanobacterium, such as cyanobacterium classified into any of the following groups based on morphology: Chlorococcales, Pleurocapsales, Oscillatoriales, Nostocales, Synechosystic or Stigonematales (See, e.g., Lindberg et al., Metab. Eng., (2010) 12(1):70-79). Illustrative plasmids or plasmid components for use in cyanobacterial cells include those described in U.S. Patent Pub. Nos. 2010/0297749 and 2009/0282545 and in Intl. Pat. Pub. No. WO 2011/034863.

Genetic Engineering Methods

Microbial cells can be engineered for fermentative 3-amino-4-hydroxybenzoic acid production using conventional techniques of molecular biology (including recombinant techniques), microbiology, cell biology, and biochemistry, which are within the skill of the art. Such techniques are explained fully in the literature, see e.g., “Molecular Cloning: A Laboratory Manual,” fourth edition (Sambrook et al., 2012); “Oligonucleotide Synthesis” (M. J. Gait, ed., 1984); “Culture of Animal Cells: A Manual of Basic Technique and Specialized Applications” (R. I. Freshney, ed., 6th Edition, 2010); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., eds., 1994); Singleton et al., Dictionary of Microbiology and Molecular Biology 2nd ed., J. Wiley & Sons (New York, N.Y. 1994).

Vectors are polynucleotide vehicles used to introduce genetic material into a cell. Vectors useful in the methods described herein can be linear or circular. Vectors can integrate into a target genome of a host cell or replicate independently in a host cell. For many applications, integrating vectors that produced stable transformants are preferred. Vectors can include, for example, an origin of replication, a multiple cloning site (MCS), and/or a selectable marker. An expression vector typically includes an expression cassette containing regulatory elements that facilitate expression of a polynucleotide sequence (often a coding sequence) in a particular host cell. Vectors include, but are not limited to, integrating vectors, prokaryotic plasmids, episomes, viral vectors, cosmids, and artificial chromosomes.

Illustrative regulatory elements that may be used in expression cassettes include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods In Enzymology 185, Academic Press, San Diego, Calif. (1990).

In some embodiments, vectors may be used to introduce systems that can carry out genome editing, such as CRISPR systems. See U.S. Patent Pub. No. 2014/0068797, published 6 Mar. 2014; see also Jinek M., et al., “A programmable dual-RNA-guided DNA endonuclease in adaptive bacterial immunity,” Science 337:816-21, 2012). In Type II CRISPR-Cas9 systems, Cas9 is a site-directed endonuclease, namely an enzyme that is, or can be, directed to cleave a polynucleotide at a particular target sequence using two distinct endonuclease domains (HNH and RuvC/RNase H-like domains). Cas9 can be engineered to cleave DNA at any desired site because Cas9 is directed to its cleavage site by RNA. Cas9 is therefore also described as an “RNA-guided nuclease.” More specifically, Cas9 becomes associated with one or more RNA molecules, which guide Cas9 to a specific polynucleotide target based on hybridization of at least a portion of the RNA molecule(s) to a specific sequence in the target polynucleotide. Ran, F. A., et al., (“In vivo genome editing using Staphylococcus aureus Cas9,” Nature 520(7546):186-91, 2015, Apr. 9], including all extended data) present the crRNA/tracrRNA sequences and secondary structures of eight Type II CRISPR-Cas9 systems. Cas9-like synthetic proteins are also known in the art (see U.S. Published Patent Application No. 2014-0315985, published 23 Oct. 2014).

Example 1 describes illustrative integration approaches for introducing polynucleotides and other genetic alterations into the genomes of C. glutamicum and S. cerevisiae cells.

Vectors or other polynucleotides can be introduced into microbial cells by any of a variety of standard methods, such as transformation, conjugation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. Transformants can be selected by any method known in the art. Suitable methods for selecting transformants are described in U.S. Patent Pub. Nos. 2009/0203102, 2010/0048964, and 2010/0003716, and International Publication Nos. WO 2009/076676, WO 2010/003007, and WO 2009/132220.

### Engineered Microbial Cells

The above-described methods can be used to produce engineered microbial cells that produce, and in certain embodiments, overproduce, 3-amino-4-hydroxybenzoic acid. Engineered microbial cells can have at least 1, 2, 3, 4, 5, 6, 7, 8, 9, 10, 20, 30, 40, 50, 60, 70, 80, 90, 100 or more genetic alterations, such as 30-100 alterations, as compared to a native microbial cell, such as any of the microbial host cells described herein. Engineered microbial cells described in the Example below have one, two, or three genetic alterations, but those of skill in the art can, following the guidance set forth herein, design microbial cells with additional alterations. In some embodiments, the engineered microbial cells have not more than 15, 14, 13, 12, 11, 10, 9, 8, 7, 6, 5, or 4 genetic alterations, as compared to a native microbial cell. In various embodiments, microbial cells engineered for 3-amino-4-hydroxybenzoic acid production can have a number of genetic alterations falling within the any of the following illustrative ranges: 1-10, 1-9, 1-8, 2-7, 2-6, 2-5, 2-4, 2-3, 3-7, 3-6, 3-5, 3-4, etc.

In some embodiments, an engineered microbial cell expresses at least two heterologous genes, e.g., a non-native 2-amino-4,5-dihydroxy-6-oxo-7-(phosphooxy) heptanoate synthase and/or or a non-native 3-amino-4-benzoic acid synthase gene. In various embodiments, the microbial cell can include and express, for example: (1) a single copy of each of these genes, (2) two or more copies of one of these genes, which can be the same or different, or (3) two or more copies of both of these genes, wherein the copies of a given gene can be the same or different. The same is true for other heterologous genes that can be introduced into the engineered microbial cell.

This engineered host cell can include at least one additional genetic alteration that increases flux through any pathway leading to the production of an immediate precursor of 3-amino-4-hydroxybenzoic acid (e.g., asparate semi-aldehyde and DHAP). As discussed above, this can be accomplished by one or more of the following: increasing the activity of upstream enzymes, expressing feedback-deregulated enzymes, reducing consumption of 3-amino-4-hydroxybenzoic acid precursors, increasing the NADPH supply, and altering the cofactor specificity of upstream pathway enzymes.

The engineered microbial cells can contain introduced genes that have a native nucleotide sequence or that differ from native. For example, the native nucleotide sequence can be codon-optimized for expression in a particular host cell. Codon optimization for a particular host can, for example, be based on the codon usage tables found at www.kazusa.or.jp/codon/. The amino acid sequences encoded by any of these introduced genes can be native or can differ from native. In various embodiments, the amino acid sequences have at least 60 percent, 70 percent, 75 percent, 80 percent, 85 percent, 90 percent, 95 percent or 100 percent amino acid sequence identity with a native amino acid sequence.

The approach described herein has been carried out in bacterial cells, namely C. glutamicum, and in yeast cells, namely S. cerevisiae. (See Examples 1-3.)

Illustrative Engineered Bacterial Cells

In certain embodiments, the engineered bacterial (e.g., C. glutamicum) cell expresses one or more non-native 2-amino-4,5-dihydroxy-6-oxo-7-(phosphooxy) heptanoate synthase(s) having at least 70 percent, 75 percent, 80 percent, 85 percent, 90 percent, 95 percent or 100 percent amino acid sequence identity with a 2-amino-4,5-dihydroxy-6-oxo-7-(phosphooxy) heptanoate synthase encoded by a Streptomyces sp. Root63 2-amino-4,5-dihydroxy-6-oxo-7-(phosphooxy) heptanoate synthase gene (e.g., SEQ ID NO:1) and one or more non-native 3-amino-4-benzoic acid synthase(s) having at least 70 percent, 75 percent, 80 percent, 85 percent, 90 percent, 95 percent or 100 percent amino acid sequence identity with a 3-amino-4-benzoic acid synthase encoded by a Saccharothrix espanaensis ATCC 51144 3-amino-4-benzoic acid synthase gene (e.g., SEQ ID NO:2).

In particular embodiments:

the non-native 2-amino-4,5-dihydroxy-6-oxo-7-(phosphooxy) heptanoate synthase includes SEQ ID NO:1; and

the non-native 3-amino-4-benzoic acid synthase includes SEQ ID NO:2.

In C. glutamicum, for example, an about 4.6 mg/L titer of 3-amino-4-hydroxybenzoic acid was achieved by overexpressing the enzymes having SEQ ID NOs:1 and 2 (see Example 1).

Illustrative Engineered Yeast Cells

In certain embodiments, the engineered yeast (e.g., S. cerevisiae) cell expresses the same enzymes as described above for illustrative engineered bacterial (e.g., C. glutamicum) cell, together with one or more non-native 2-amino-4,5-dihydroxy-6-oxo-7-(phosphooxy) heptanoate synthase(s) having at least 70 percent, 75 percent, 80 percent, 85 percent, 90 percent, 95 percent or 100 percent amino acid sequence identity with a 2-amino-4,5-dihydroxy-6-oxo-7-(phosphooxy) heptanoate synthase from Bacteriodetes bacterium GWE2_32_14 (e.g., SEQ ID NO:3) and one or more non-native 3-amino-4-benzoic acid synthase(s) having at least 70 percent, 75 percent, 80 percent, 85 percent, 90 percent, 95 percent or 100 percent amino acid sequence identity with a 3-amino-4-benzoic acid synthase from Streptomyces griseus (SEQ ID NO:4).

In particular embodiments:

the non-native 2-amino-4,5-dihydroxy-6-oxo-7-(phosphooxy) heptanoate synthase includes SEQ ID NO:3; and

the non-native 3-amino-4-benzoic acid synthase comprises SEQ ID NO:4.

In an illustrative embodiment, a titer of about 753 μg/L was achieved after engineering S. cerevisiae to express a 2-amino-4,5-dihydroxy-6-oxo-7-(phosphooxy) heptanoate synthase from Bacteriodetes bacterium GWE2_32_14 and a 3-amino-4-benzoic acid synthase from Streptomyces griseus.

In certain embodiments, the engineered yeast (e.g., S. cerevisiae) cell expresses the same enzymes as described above for illustrative engineered bacterial (e.g., C. glutamicum) cell, together with one or more non-native 2-amino-4,5-dihydroxy-6-oxo-7-(phosphooxy) heptanoate synthase(s) having at least 70 percent, 75 percent, 80 percent, 85 percent, 90 percent, 95 percent or 100 percent amino acid sequence identity with a 2-amino-4,5-dihydroxy-6-oxo-7-(phosphooxy) heptanoate synthase from Streptomyces thermoautotrophicus (e.g., SEQ ID NO:5) and one or more non-native 3-amino-4-benzoic acid synthase(s) having at least 70 percent, 75 percent, 80 percent, 85 percent, 90 percent, 95 percent or 100 percent amino acid sequence identity with a 3-amino-4-benzoic acid synthase from Streptomyces griseus (SEQ ID NO:4).

In particular embodiments:

the non-native 2-amino-4,5-dihydroxy-6-oxo-7-(phosphooxy) heptanoate synthase includes SEQ ID NO:5;

the non-native 3-amino-4-benzoic acid synthase comprises SEQ ID NO:4.

In an illustrative embodiment, a titer of about 3.3 mg/L was achieved after engineering S. cerevisiae to express a 2-amino-4,5-dihydroxy-6-oxo-7-(phosphooxy) heptanoate synthase from Streptomyces thermoautotrophicus and a 3-amino-4-benzoic acid synthase from Streptomyces griseus.

### Culturing of Engineered Microbial Cells

Any of the microbial cells described herein can be cultured, e.g., for maintenance, growth, and/or 3-amino-4-hydroxybenzoic acid production.

In some embodiments, the cultures are grown to an optical density at 600 nm of 10-500, such as an optical density of 50-150.

In various embodiments, the cultures include produced 3-amino-4-hydroxybenzoic acid at titers of at least 10, 20, 30, 40, 50, 75, 100, 200, 300, 400, 500, 600, 700, 800, or 900 μg/L or at least 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 110, 120, 130, 140, or 150 mg/L. In various embodiments, the titer is in the range of 50 μg/L to 100 mg/L, 75 μg/L to 75 mg/L, 100 μg/L to 50 gm/L, 200 μg/L to 25 gm/L, 300 μg/L to 10 gm/L, 350 μg/L to 5 gm/L or any range bounded by any of the values listed above.

Culture Media

Microbial cells can be cultured in any suitable medium including, but not limited to, a minimal medium, i.e., one containing the minimum nutrients possible for cell growth. Minimal medium typically contains: (1) a carbon source for microbial growth; (2) salts, which may depend on the particular microbial cell and growing conditions; and (3) water. Suitable media can also include any combination of the following: a nitrogen source for growth and product formation, a sulfur source for growth, a phosphate source for growth, metal salts for growth, vitamins for growth, and other cofactors for growth.

Any suitable carbon source can be used to cultivate the host cells. The term “carbon source” refers to one or more carbon-containing compounds capable of being metabolized by a microbial cell. In various embodiments, the carbon source is a carbohydrate (such as a monosaccharide, a disaccharide, an oligosaccharide, or a polysaccharide), or an invert sugar (e.g., enzymatically treated sucrose syrup). Illustrative monosaccharides include glucose (dextrose), fructose (levulose), and galactose; illustrative oligosaccharides include dextran or glucan, and illustrative polysaccharides include starch and cellulose. Suitable sugars include C6 sugars (e.g., fructose, mannose, galactose, or glucose) and C5 sugars (e.g., xylose or arabinose). Other, less expensive carbon sources include sugar cane juice, beet juice, sorghum juice, and the like, any of which may, but need not be, fully or partially deionized.

The salts in a culture medium generally provide essential elements, such as magnesium, nitrogen, phosphorus, and sulfur to allow the cells to synthesize proteins and nucleic acids.

Minimal medium can be supplemented with one or more selective agents, such as antibiotics.

To produce 3-amino-4-hydroxybenzoic acid, the culture medium can include, and/or is supplemented during culture with, glucose and/or a nitrogen source such as urea, an ammonium salt, ammonia, or any combination thereof.

Culture Conditions

Materials and methods suitable for the maintenance and growth of microbial cells are well known in the art. See, for example, U.S. Pub. Nos. 2009/0203102, 2010/0003716, and 2010/0048964, and International Pub. Nos. WO 2004/033646, WO 2009/076676, WO 2009/132220, and WO 2010/003007, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass.

In general, cells are grown and maintained at an appropriate temperature, gas mixture, and pH (such as about 20° C. to about 37° C., about 6% to about 84% C02, and a pH between about 5 to about 9). In some aspects, cells are grown at 35° C. In certain embodiments, such as where thermophilic bacteria are used as the host cells, higher temperatures (e.g., 50° C.-75° C.) may be used. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the particular cell.

Standard culture conditions and modes of fermentation, such as batch, fed-batch, or continuous fermentation that can be used are described in U.S. Publ. Nos. 2009/0203102, 2010/0003716, and 2010/0048964, and International Pub. Nos. WO 2009/076676, WO 2009/132220, and WO 2010/003007. Batch and Fed-Batch fermentations are common and well known in the art, and examples can be found in Brock, Biotechnology: A Textbook of Industrial Microbiology, Second Edition (1989) Sinauer Associates, Inc.

In some embodiments, the cells are cultured under limited sugar (e.g., glucose) conditions. In various embodiments, the amount of sugar that is added is less than or about 105% (such as about 100%, 90%, 80%, 70%, 60%, 50%, 40%, 30%, 20%, or 10%) of the amount of sugar that can be consumed by the cells. In particular embodiments, the amount of sugar that is added to the culture medium is approximately the same as the amount of sugar that is consumed by the cells during a specific period of time. In some embodiments, the rate of cell growth is controlled by limiting the amount of added sugar such that the cells grow at the rate that can be supported by the amount of sugar in the cell medium. In some embodiments, sugar does not accumulate during the time the cells are cultured. In various embodiments, the cells are cultured under limited sugar conditions for times greater than or about 1, 2, 3, 5, 10, 15, 20, 25, 30, 35, 40, 50, 60, or 70 hours or even up to about 5-10 days. In various embodiments, the cells are cultured under limited sugar conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited sugar conditions can allow more favorable regulation of the cells.

In some aspects, the cells are grown in batch culture. The cells can also be grown in fed-batch culture or in continuous culture. Additionally, the cells can be cultured in minimal medium, including, but not limited to, any of the minimal media described above. The minimal medium can be further supplemented with 1.0% (w/v) glucose (or any other six-carbon sugar) or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. In some cultures, significantly higher levels of sugar (e.g., glucose) are used, e.g., at least 10% (w/v), 20% (w/v), 30% (w/v), 40% (w/v), 50% (w/v), 60% (w/v), 70% (w/v), or up to the solubility limit for the sugar in the medium. In some embodiments, the sugar levels falls within a range of any two of the above values, e.g.: 0.1-10% (w/v), 1.0-20% (w/v), 10-70% (w/v), 20-60% (w/v), or 30-50% (w/v). Furthermore, different sugar levels can be used for different phases of culturing. For fed-batch culture (e.g., of S. cerevisiae or C. glutamicum), the sugar level can be about 100-200 g/L (10-20% (w/v)) in the batch phase and then up to about 500-700 g/L (50-70% in the feed).

Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), or 0.02% (w/v) yeast extract. In some cultures, significantly higher levels of yeast extract can be used, e.g., at least 1.5% (w/v), 2.0% (w/v), 2.5% (w/v), or 3% (w/v). In some cultures (e.g., of S. cerevisiae or C. glutamicum), the yeast extract level falls within a range of any two of the above values, e.g.: 0.5-3.0% (w/v), 1.0-2.5% (w/v), or 1.5-2.0% (w/v).

### 3-Amino-4-Hydroxybenzoic Acid Production and Recovery

Any of the methods described herein may further include a step of recovering 3-amino-4-hydroxybenzoic acid. In some embodiments, the produced 3-amino-4-hydroxybenzoic acid contained in a so-called harvest stream is recovered/harvested from the production vessel. The harvest stream may include, for instance, cell-free or cell-containing aqueous solution coming from the production vessel, which contains 3-amino-4-hydroxybenzoic acid as a result of the conversion of production substrate by the resting cells in the production vessel. Cells still present in the harvest stream may be separated from the 3-amino-4-hydroxybenzoic acid by any operations known in the art, such as for instance filtration, centrifugation, decantation, membrane crossflow ultrafiltration or microfiltration, tangential flow ultrafiltration or microfiltration or dead-end filtration. After this cell separation operation, the harvest stream is essentially free of cells.

Further steps of separation and/or purification of the produced 3-amino-4-hydroxybenzoic acid from other components contained in the harvest stream, i.e., so-called downstream processing steps may optionally be carried out. These steps may include any means known to a skilled person, such as, for instance, concentration, extraction, crystallization, precipitation, adsorption, ion exchange, and/or chromatography. Any of these procedures can be used alone or in combination to purify 3-amino-4-hydroxybenzoic acid. Further purification steps can include one or more of, e.g., concentration, crystallization, precipitation, washing and drying, treatment with activated carbon, ion exchange, nanofiltration, and/or re-crystallization. The design of a suitable purification protocol may depend on the cells, the culture medium, the size of the culture, the production vessel, etc. and is within the level of skill in the art.

The following examples are given for the purpose of illustrating various embodiments of the disclosure and are not meant to limit the present disclosure in any fashion. Changes therein and other uses which are encompassed within the spirit of the disclosure, as defined by the scope of the claims, will be identifiable to those skilled in the art.

### Example 1—Construction and Selection of Strains of Corynebacteria glutamicum Engineered to Produce 3-Amino-4-Hydroxybenzoic Acid

Plasmid/DNA Design

All strains tested for this work were transformed with plasmid DNA designed using proprietary software. Plasmid designs were specific to each of the host organisms engineered in this work. The plasmid DNA was physically constructed by a standard DNA assembly method. This plasmid DNA was then used to integrate metabolic pathway inserts by one of two host-specific methods, each described below.

C. glutamicum and B. subtilis Pathway Integration

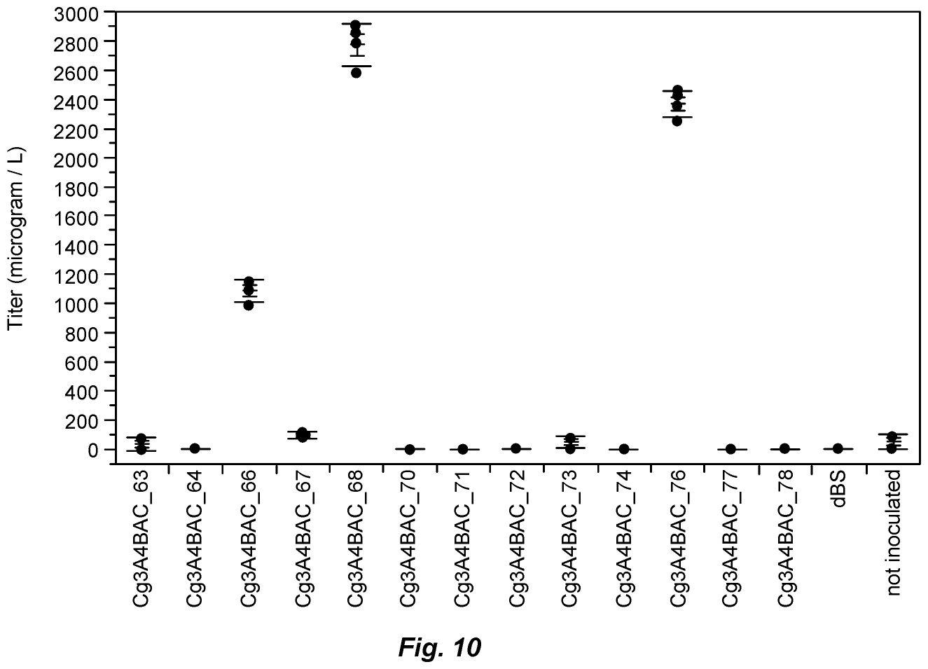

A “loop-in, single-crossover” genomic integration strategy has been developed to engineer C. glutamicum and B. subtilis strains. FIG. 10 illustrates genomic integration of loop-in only and loop-in/loop-out constructs and verification of correct integration via colony PCR. Loop-in only constructs (shown under the heading “Loop-in”) contained a single 2-kb homology arm (denoted as “integration locus”), a positive selection marker (denoted as “Marker”)), and gene(s) of interest (denoted as “promoter-gene-terminator”). A single crossover event integrated the plasmid into the C. glutamicum or B. subtilis chromosome. Integration events are stably maintained in the genome by growth in the presence of antibiotic (25 μg/ml kanamycin). Correct genomic integration in colonies derived from loop-in integration were confirmed by colony PCR with UF/IR and DR/IF PCR primers.

Loop-in, loop-out constructs (shown under the heading “Loop-in, loop-out) contained two 2-kb homology arms (5′ and 3′ arms), gene(s) of interest (arrows), a positive selection marker (denoted “Marker”), and a counter-selection marker. Similar to “loop-in” only constructs, a single crossover event integrated the plasmid into the chromosome. Note: only one of two possible integrations is shown here. Correct genomic integration was confirmed by colony PCR and counter-selection was applied so that the plasmid backbone and counter-selection marker could be excised. This results in one of two possibilities: reversion to wild-type (lower left box) or the desired pathway integration (lower right box). Again, correct genomic loop-out is confirmed by colony PCR. (Abbreviations: Primers: UF=upstream forward, DR=downstream reverse, IR=internal reverse, IF=internal forward.)

S. cerevisiae Pathway Integration

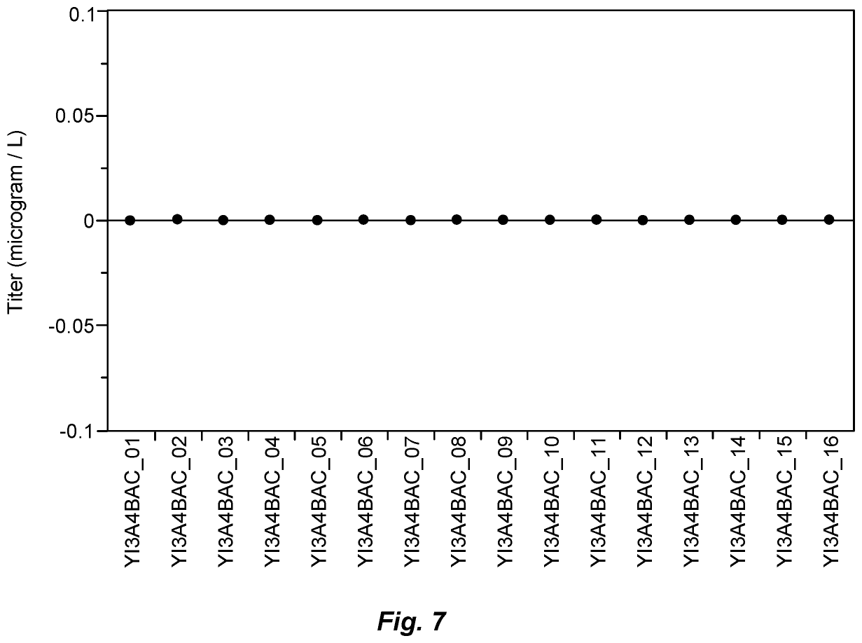

A “split-marker, double-crossover” genomic integration strategy has been developed to engineer S. cerevisiae strains. FIG. 7 illustrates genomic integration of complementary, split-marker plasmids and verification of correct genomic integration via colony PCR in S. cerevisiae. Two plasmids with complementary 5′ and 3′ homology arms and overlapping halves of a URA3 selectable marker (direct repeats shown by the hashed bars) were digested with meganucleases and transformed as linear fragments. A triple-crossover event integrated the desired heterologous genes into the targeted locus and re-constituted the full URA3 gene. Colonies derived from this integration event were assayed using two 3-primer reactions to confirm both the 5′ and 3′ junctions (UF/IF/wt-R and DR/IF/wt-F). For strains in which further engineering is desired, the strains can be plated on 5-FOA plates to select for the removal of URA3, leaving behind a small single copy of the original direct repeat. This genomic integration strategy can be used for gene knock-out, gene knock-in, and promoter titration in the same workflow.

Cell Culture

The workflow established for S. cerevisiae involved a hit-picking step that consolidated successfully built strains using an automated workflow that randomized strains across the plate. For each strain that was successfully built, up to four replicates were tested from distinct colonies to test colony-to-colony variation and other process variation. If fewer than four colonies were obtained, the existing colonies were replicated so that at least four wells were tested from each desired genotype.

The colonies were consolidated into 96-well plates with selective medium (SD-ura for S. cerevisiae) and cultivated for two days until saturation and then frozen with 16.6% glycerol at −80° C. for storage. The frozen glycerol stocks were then used to inoculate a seed stage in minimal media with a low level of amino acids to help with growth and recovery from freezing. The seed plates were grown at 30° C. for 1-2 days. The seed plates were then used to inoculate a main cultivation plate with minimal medium and grown for 48-88 hours. Plates were removed at the desired time points and tested for cell density (OD600), viability and glucose, supernatant samples stored for LC-MS analysis for product of interest.

Cell Density

Cell density was measured using a spectrophotometric assay detecting absorbance of each well at 600 nm. Robotics were used to transfer fixed amounts of culture from each cultivation plate into an assay plate, followed by mixing with 175 mM sodium phosphate (pH 7.0) to generate a 10-fold dilution. The assay plates were measured using a Tecan M1000 spectrophotometer and assay data uploaded to a LIMS database. A non-inoculated control was used to subtract background absorbance. Cell growth was monitored by inoculating multiple plates at each stage, and then sacrificing an entire plate at each time point.

To minimize settling of cells while handling large number of plates (which could result in a non-representative sample during measurement) each plate was shaken for 10-15 seconds before each read. Wide variations in cell density within a plate may also lead to absorbance measurements outside of the linear range of detection, resulting in underestimate of higher OD cultures. In general, the tested strains so far have not varied significantly enough for this be a concern.

Liquid-Solid Separation

To harvest extracellular samples for analysis by LC-MS, liquid and solid phases were separated via centrifugation. Cultivation plates were centrifuged at 2000 rpm for 4 minutes, and the supernatant was transferred to destination plates using robotics. 75 μL of supernatant was transferred to each plate, with one stored at 4° C., and the second stored at 80° C. for long-term storage.

First-Round Genetic Engineering Results in Corynebacteria glutamicum and Saccharomyces cerevisiae

Initially, Corynebacteria glutamicum and Saccharomyces cerevisiae were engineered for 3-amino-4-hydroxy-benzoic acid production by addition of 2-amino-4,5-dihydroxy-6-oxo-7-(phosphooxy)heptanoate synthase and a 3-amino-4-hydroxybenzoic acid synthase.

The best-performing C. glutamicum strain in the first round of engineering produced 4.6 mg/L 3-amino-4-hydroxybenzoic acid, and this strain expressed 2-amino-4,5-dihydroxy-6-oxo-7-(phosphooxy)heptanoate synthase from Streptomyces sp. Root63 (UniProt ID A0A0Q8F363) and 3-amino-4-benzoic acid synthase from Saccharothrix espanaensis ATCC 51144 (UniProt ID K0JXI9). Five additional strains also produced 3-amino-4-hydroxy-benzoic acid (see FIG. 2 and Table 1).

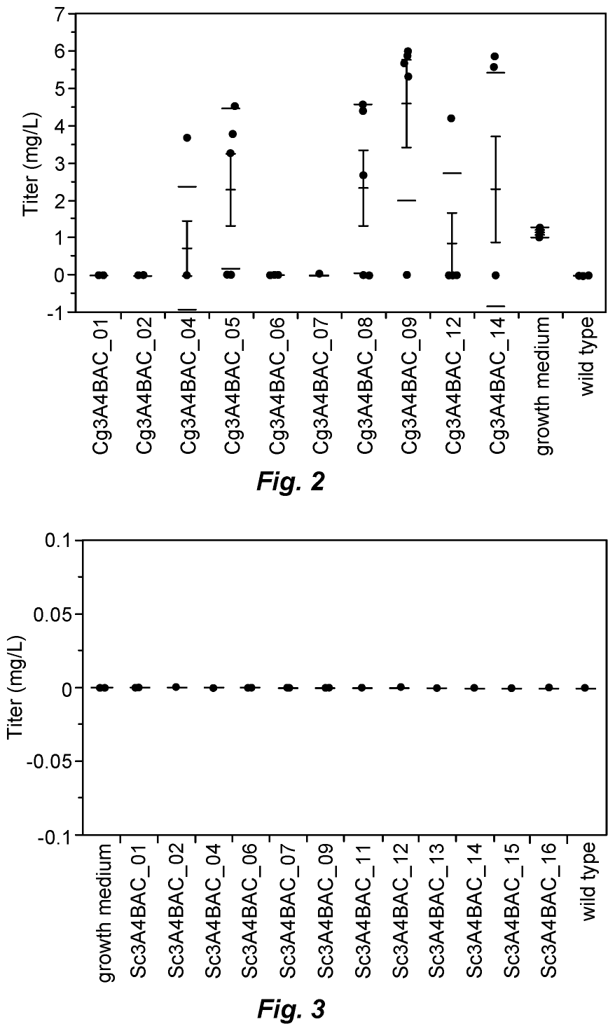

No detectable 3-amino-4-hydroxy-benzoic acid was produced in S. cerevisiae strains which tested the same enzymes. (FIG. 3 and Table 2).

Second-Round Genetic Engineering Results in Corynebacteria glutamicum

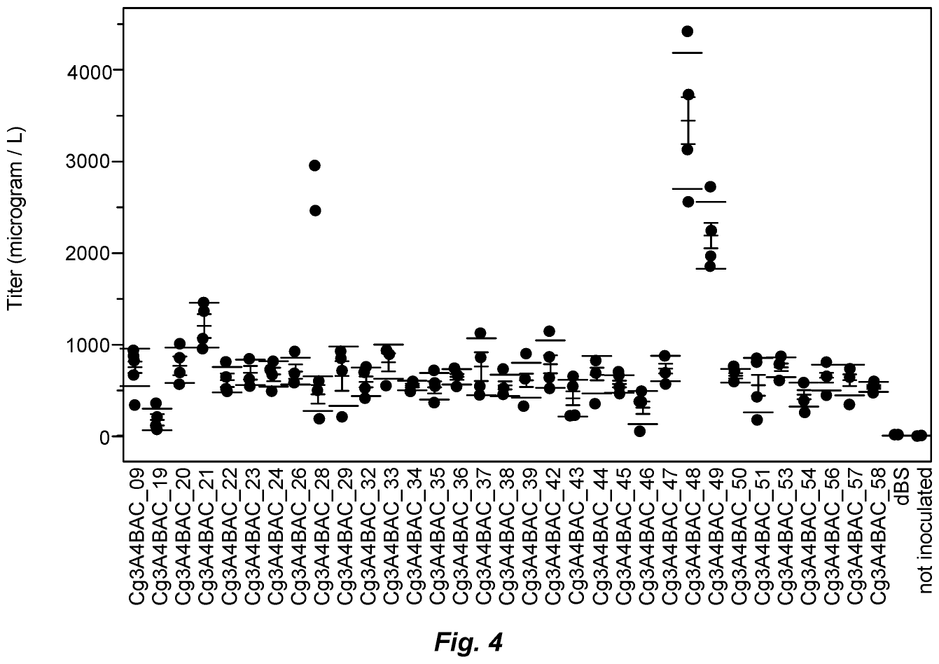

A second round of genetic engineering was carried out in Corynebacteria glutamicum. The strains tested contained the best enzymes from first round: 2-amino-4,5-dihydroxy-6-oxo-7-(phosphooxy)heptanoate synthase (UniProt ID A0A0Q8F363) from Streptomyces sp. Root63, and 3-amino-4-benzoic acid synthase (UniProt ID K0JXI9) from Saccharothrix espanaensis ATCC 51144, as well as the further genetic alterations indicated in Table 3 below (see FIG. 4 for results). The best-performing strain produced 3.5 mg/L, and this strain expressed one additional enzyme (in addition to the two enzymes from the first round): 3-amino-4-benzoic acid synthase from Kutzneria albida DSM 43870 (UniProt ID W5WBR4).

### Example 2—Host Evaluation for Production of 3-Amino-4-Hydroxybenzoic Acid

3-Amino-4-hydroxy-benzoic acid was produced in Corynebacterium glutamicum strains (FIG. 10, Table 7) that expressed host evaluation designs. The best-performing strain produced 2.8 mg/L, and this strain expressed 2-amino-4,5-dihydroxy-6-oxo-7-(phosphooxy)heptanoate synthase from Bacillus cereus (NCBI protein identifier CUB39904.1) and 3-amino-4-benzoic acid synthase from Bacillus anthracis (UniProt ID A0A1T3V8D3), and aspartokinase harboring the amino acid substitution Q298G from C. glutamicum where all three DNA sequences were codon optimized for Yarrowia lipolytica.

3-Amino-4-hydroxy-benzoic acid was produced in Saccharomyces cerevisiae strains (FIG. 9, Table 6) that expressed host evaluation designs. The best-performing strain produced 24 μg/L and expressed 2-amino-4,5-dihydroxy-6-oxo-7-(phosphooxy)heptanoate synthase from Streptomyces sp. Root63 (UniProt ID A0A0Q8F363) and 3-amino-4-benzoic acid synthase from Saccharothrix espanaensis ATCC 51144 (UniProt ID K0JXI9), where both DNA sequences were codon optimized for Corynebacteria glutamicum.

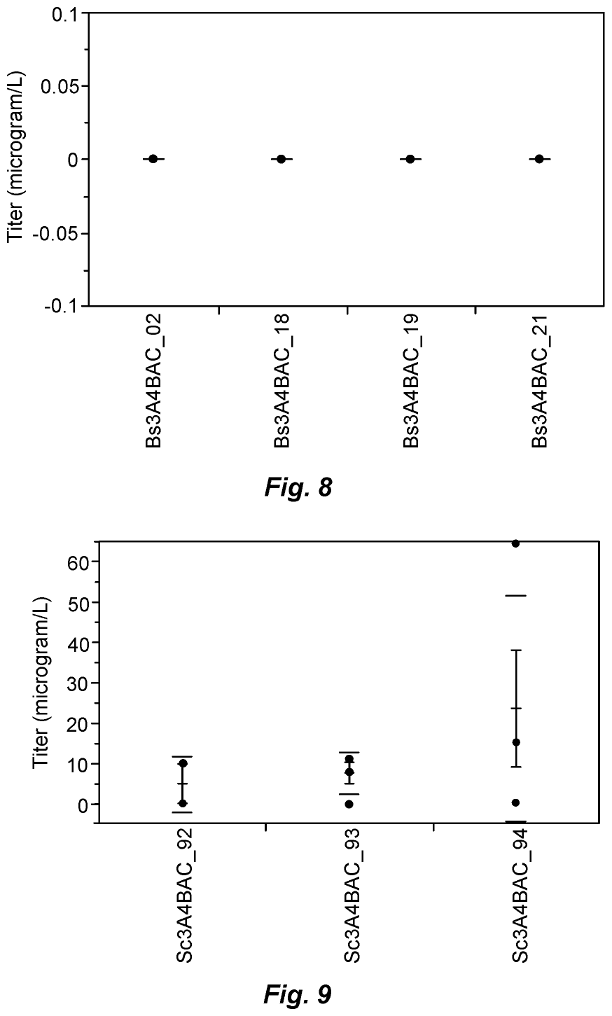

There was no detectable production of 3-amino-4-hydroxy-benzoic acid in Yarrowia lipolytica (FIG. 7, Table 4) or Bacillus subtillus (FIG. 8, Table 5).

### Example 3—Improving 3-Amino-4-Hydroxybenzoic Acid Production in Saccharomyces cerevisiae

Two heterologous enzymes, 3-amino-4-hydroxybenzoic acid synthase and 2-amino-4,5-dihydroxy-6-oxo-7-(phosphooxy)heptanoate synthase were previously tested in Saccharomyces cerevisiae (host-evaluation round of genetic engineering). In all cases, the enzymes were tested in combinations from different species. For strain Sc3A4BAC_09, the two enzymes tested were each found to be active in Corynebacteria glutamicum, yet there was no 3-amino-4-hydroxybenzoic acid detected in S. cerevisiae extracellular material for this strain, or any of the other S. cerevisiae strains.

The substrates for 2-amino-4,5-dihydroxy-6-oxo-7-(phosphonooxy)heptanoate synthase are aspartate semi-aldehyde and the glycolytic metabolite, dihydroxyacetone phosphate (DHAP). The aspartate aminotransferases (an upstream pathway enzyme leading to aspartate semi-aldehyde) in S. cerevisiae localize to the peroxisome, AAT1, or the mitochondria, AAT2, when grown in fat carbon sources. Therefore, we considered that expression of cytosolic enzymes may improve production of 3-amino-4-hydroxybenzoic acid.

The metabolite precursor to aspartate is oxaloacetate, which is also a precursor to malate. Zelle et al. were able to improve malate production in Saccharomyces CEN.PK by expressing pyruvate carboxylase and a modified malate dehydrogenase (normally found in the peroxiosome, truncation of the 3 C-terminal amino acids of malate dehydrogenase resulted in cytosolic expression of the enzyme) [7]. Production of 3-amino-4-hydroxybenzoic acid can be improved by expressing a cytosolic pyruvate carboxylase in combination with a cytosolic aspartate aminotransferase, and a feedback-deregulated aspartate kinase. Pyruvate carboxylase catalyzes phosphoenolpyruvate (PEP) conversion to oxaloacetate via pyruvate, while producing zero ATP molecules overall. Replacement of pyruvate carboxylase with PEP carboxylase affords more efficient production of oxaloacetate, since PEP carboxylase converts PEP directly to oxaloacetate, while producing an ATP molecule. The additional ATP in PEP carboxylase-containing strains can improve 3-amino-4-hydroxybenzoic acid production.

3-Amino-4-hydroxybenzoic acid production was achieved in S. cerevisiae strains (see FIG. 11, Table 8) which already expressed the 3-amino-4-hydroxybenzoic acid pathway (2-amino-4,5-dihydroxy-6-oxo-7-(phosphooxy)heptanoate synthase from Streptomyces sp. Root63 (UniProt ID A0A0Q8F363) and 3-amino-4-benzoic acid synthase from Saccharothrix espanaensis ATCC 51144 (UniProt ID K0JXI9), where both DNA sequences were codon-optimized using a modified codon table for Saccharomyces cerevisiae and Corynebacteria glutamicum) upon the addition of aspartokinase (UniProt ID P26512) from Corynebacterium glutamicum ATCC 13032 harboring the amino acid substitution Q298G, with phosphoenolpyruvate carboxylase from C. glutamicum ATCC 13032 (UniProt ID P12880), or upon the addition of aspartokinase (UniProt ID P26512) from C. glutamicum ATCC 13032 harboring the amino acid substitution Q298G, aspartate-semialdehyde dehydrogenase from C. glutamicum ATCC 13032 (UniProt ID P0C1D8), and aspartate aminotransferase (UniProt ID P00509) from Escherichia coli K12. It was also found that expression of PEP synthase from E. coli (UniProt ID P23538) (along with 2-amino-4,5-dihydroxy-6-oxo-7-(phosphooxy)heptanoate synthase from Streptomyces sp. Root63 (UniProt ID A0A0Q8F363) and 3-amino-4-benzoic acid synthase from Saccharothrix espanaensis ATCC 51144 (UniProt ID K0JXI9), where both DNA sequences were codon optimized for C. glutamicum) enabled 3-amino-4-hydroxybenzoic acid production in S. cerevisiae.

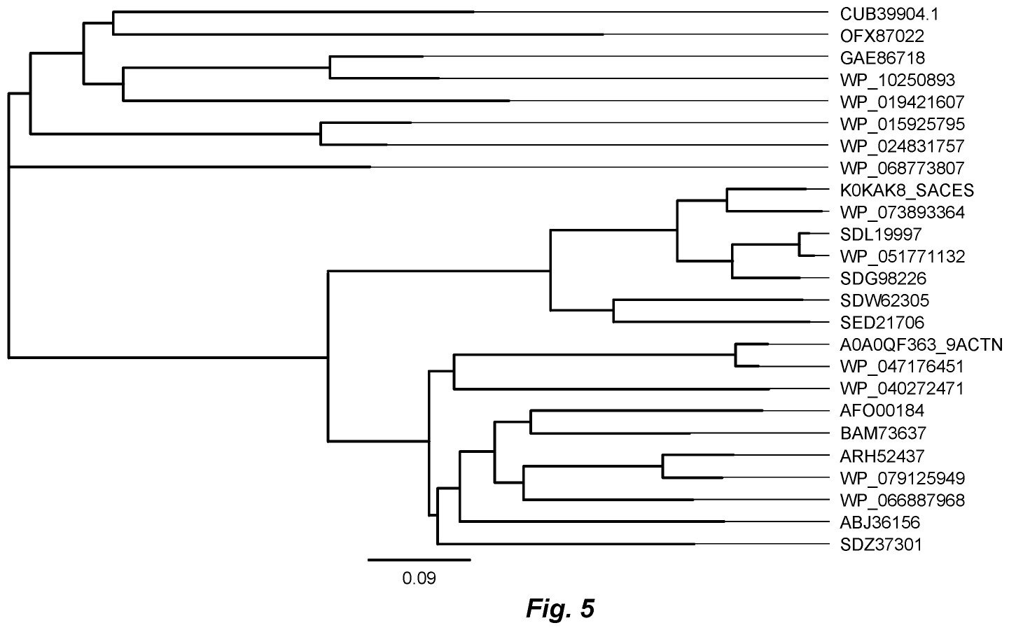

In addition, 3-amino-4-hydroxybenzoic acid synthase (UniProt ID A0JC76) was tested with several different homologs of 2-amino-4,5-dihydroxy-6-oxo-7-(phosphooxy)heptanoate synthase to identify more active enzymes. A comparison of the protein amino acid sequences tested is shown in a tree (FIG. 5). The homologs were identified by BlastP search using 3 enzymes: UniProt ID KOKAK8, UniProt ID A0A0Q8F363, NCBI protein identifier CUB39904.1 (not originally tested but sequence discovered by inspection of the local genome of Bacillus cereus, because it was located next to the 3-amino-4-hydroxybenzoic acid synthase homolog UniProt ID A0A0K6LJE3 and it shared homology to other 2-amino-4,5-dihydroxy-6-oxo-7-(phosphooxy)heptanoate synthases). Several enzymes produced titer in S. cerevisiae (see Table 8).

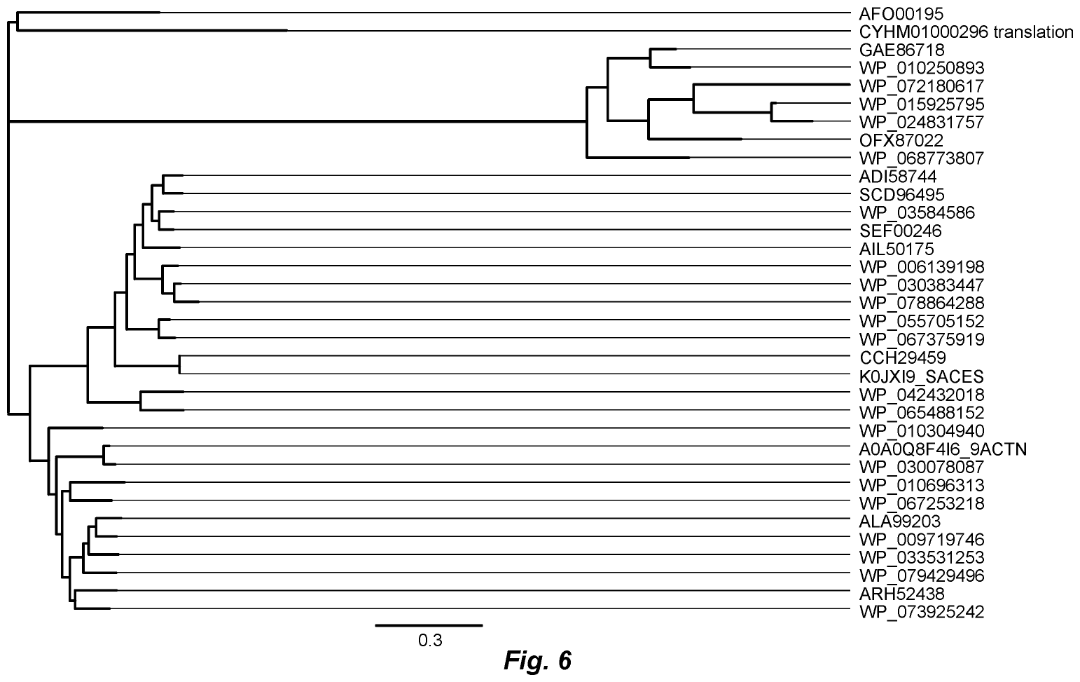

In addition, 2-amino-4,5-dihydroxy-6-oxo-7-(phosphooxy)heptanoate synthase (UniProt ID A0JC77) was tested with several different homologs of 3-amino-4-hydroxybenzoic acid synthase to identify more active enzymes. A comparison of the protein amino acid sequences tested is shown in a tree (FIG. 6). The homologs were identified by BlastP search using 3 enzymes: UniProt ID K0JXI9, UniProt ID A0A0K6LJE3, and UniProt A0A0Q8F4I6 (not originally tested but sequence discovered by inspection of the local genome of Streptomyces sp. Root63, because it was located next to the 2-amino-4,5-dihydroxy-6-oxo-7-(phosphooxy)heptanoate synthase homolog UniProt ID A0A0Q8F363 and it shared homology to other 3-amino-4-hydroxybenzoic acid synthases). Several enzymes identified in the BlastP search produced titer in S. cerevisiae (see Table 8).

### Example 4—Further Improvement of 3-Amino-4-Hydroxybenzoic Acid Production

Approaches to further improve 3-amino-4-hydroxybenzoic acid production include the following:

Increase the DHAP intracellular concentration: Blocking the pathway from DHAP to glycerol is generally not favorable to Saccharomyces because this pathway is a route to “dump” excess redox by producing glycerol. But, by “backing up” the pathway from DHAP to glycerol, the cytosolic concentration of DHAP may increase, and could be beneficial if DHAP availability is limiting to 3-amino-4-hydroxybenzoic acid production.

Decrease expression or activity of glycerol-3-phosphate dehydrogenase (EC 1.1.5.3) (in Saccharomyces cerevisiae: GPD1=YDL022W, and GPD2=YOL059W). DHAP can be consumed by glycerol-3-phosphate dehydrogenase.

Truncate Fps1 which controls glycerol export in Saccharomyces cerevisiae (in Saccharomyces cerevisiae FPS1=YLL043W]: DHAP is reduced to glycerol-phosphate which is subsequently converted to glycerol [8-10].

Install a “slower” TPI, triose phosphate isomerase (EC 5.3.1.1), which interconverts DHAP and glyceraldehyde-3-phosphate (UniProt ID P00942) from Saccharomyces cerevisiae S288c harboring either amino acid substitution I170V or I170T, and lower expression of the native triose phosphate isomerase. Since the pathway for 3-amino-4-hydroxybenzoate uses the upper glycolytic metabolite, DHAP, and aspartate semi-aldehyde, control of flux at this step enables balancing the availability of both precursors. The native TPI enzyme has a long lifetime in the cell, and the two enzyme variants increase turnover of the protein-enabling modulation of TPI activity levels.

Decrease expression or activity or delete glycerol-3-phosphate/dihydroxyacetone phosphate acyltransferase (EC 2.3.1.15/EC 2.3.1.42) (In Saccharomyces cerevisiae GPT2=YKR067W) which consumes DHAP and glycerol-3-phosphate.

Decrease pyruvate dehydrogenase (PDH) activity in ensure more flux to the 3-amino-4-hydroxybenzoic acid pathway precursor aspartate semi-aldehyde.

Strain modifications that improve NADPH cofactor availability will improve 3-amino-4-hydroxy-benzoic acid production: install heterologous NADP+ reducing glyceraldehyde-3 phosphate dehydrogenases, GapA, GapN, and lower expression of the native GAPDHs, which reduce NAD+ to NADH. In Saccharomyces cerevisiae, these enzymes are encoded by: TDH1=YJL052W, TDH2=YJR009C, TDH3=YGR192C. This modification may be helpful in order to realize a benefit from expressing the heterologous NADP⁺ reducing glyceraldehyde-3 phosphate dehydrogenases. Alternatively, pentose phosphate pathway flux can be improved through deregulation, by engineering zwf harboring the amino acid substitution A243T (Becker J1, Klopprogge C, Herold A, Zelder O, Bolten C J, Wittmann C. Metabolic flux engineering of L-lysine production in Corynebacterium glutamicum—over expression and modification of G6P dehydrogenase. J Biotechnol. 2007 Oct. 31; 132(2):99-109. Epub 2007 Jun. 6.). An additional alternative solution is also to replace the NADPH-utilizing aspartate semi-aldehyde dehydrogenase with an NADH-utilizing enzyme (Wu et al. Efficient mining of natural NADH-utilizing dehydrogenases enables systematic cofactor engineering of lysine synthesis pathway of Corynebacterium glutamicum. Metabolic Engineering (2019) 52:77-86.). Each of these solutions will also increase the theoretical maximum yield for 3-amino-4-hydroxybenzoic acid.

If the host organism has PEP carboxykinase (PEPCK) delete it from the organism. The reaction catalyzes the reverse reaction of PEP carboxylase.

Lower expression of homoserine dehydrogenase (EC 1.1.1.3), which consumes aspartate semi-aldehyde, a precursor metabolite to 3-amino-4-hydroxybenzoic acid.

Lower expression of the lysine biosynthesis enzyme such as 4-hydroxy-tetrahydrodipicolinate synthase (EC 4.3.3.7), which consumes aspartate semialdehyde.

Improve nitrogen availability for aspartate semi-aldehyde biosynthesis by increasing activity or expression of: glutamate synthase (EC 1.4.1.14), glutamine synthetase (EC 6.3.1.2) and/or glutamate dehydrogenase (EC 1.4.1.2).

Finally, the more active enzymes discovered in Saccharomyces cerevisiae (FIGS. 9 and 11, Tables 6 and 8) described above may be tested in Yarrowia lipolytica and Bacillus subtillus to enable production in those hosts.

### Genetic Engineering Results Tables

TABLE 1
First-round genetic engineering in Corynebacteria glutamicum

| | | | | | E1 Codon |
|---|---|---|---|---|---|
| | Titer | E1 | Enzyme 1 - | Enzyme 1 - | Optimization |
| Strain name | (μg/L) | Uniprot ID | activity name | source organism | Abbrev. |
| Cg3A4BAC_01 | 0 | A0A166BTP9 | 2-amino-4,5-dihydroxy-6- | Methanobrevibacter | modified |
| | | | oxo-7- | oralis | Cg codon |
| | | | (phosphooxy)heptanoate | | usage |
| | | | synthase | | |
| Cg3A4BAC_02 | 0 | A0A166CVJ4 | 2-amino-4,5-dihydroxy-6- | Methanobrevibacter | modified |
| | | | oxo-7- | cuticularis | Cg codon |
| | | | (phosphooxy)heptanoate | | usage |
| | | | synthase | | |
| Cg3A4BAC_04 | 734 | A0A166D2B9 | 2-amino-4,5-dihydroxy-6- | Methanobrevibacter | modified |
| | | | oxo-7- | filiformis | Cg codon |
| | | | (phosphooxy)heptanoate | | usage |
| | | | synthase | | |
| Cg3A4BAC_05 | 2313 | A0JC77 | 2-amino-4,5-dihydroxy-6- | Streptomyces | modified |
| | | | oxo-7- | griseus | Cg codon |
| | | | (phosphooxy)heptanoate | | usage |
| | | | synthase | | |
| Cg3A4BAC_06 | 0 | A0A0N8G7L8 | 2-amino-4,5-dihydroxy-6- | Streptomyces | modified |
| | | | oxo-7- | anulatus | Cg codon |
| | | | (phosphooxy)heptanoate | (Streptomyces | usage |
| | | | synthase | chrysomallus) | |
| Cg3A4BAC_07 | 0 | A0A0S3TVT4 | 2-amino-4,5-dihydroxy-6- | Streptomyces | modified |
| | | | oxo-7- | cremeus | Cg codon |
| | | | (phosphooxy)heptanoate | | usage |
| | | | synthase | | |
| Cg3A4BAC_08 | 2330 | A0A0M4DD67 | 2-amino-4,5-dihydroxy-6- | Streptomyces | modified |
| | | | oxo-7- | pristinaespiralis | Cg codon |
| | | | (phosphooxy)heptanoate | | usage |
| | | | synthase | | |
| Cg3A4BAC_09 | 4573 | A0A0Q8F363 | 2-amino-4,5-dihydroxy-6- | Streptomyces | modified |
| | | | oxo-7- | sp. Root63 | Cg codon |
| | | | (phosphooxy)heptanoate | | usage |
| | | | synthase | | |
| Cg3A4BAC_10 | | A0A0P6UHB3 | 2-amino-4,5-dihydroxy-6- | Streptomyces | modified |
| | | | oxo-7- | anulatus | Cg codon |
| | | | (phosphooxy)heptanoate | (Streptomyces | usage |
| | | | synthase | chrysomallus) | |
| Cg3A4BAC_11 | | W5W353 | 2-amino-4,5-dihydroxy-6- | Kutzneria albida | modified |
| | | | oxo-7- | DSM 43870 | Cg codon |
| | | | (phosphooxy)heptanoate | | usage |
| | | | synthase | | |
| Cg3A4BAC_12 | 841 | A0A0M9XA57 | 2-amino-4,5-dihydroxy-6- | Streptomyces | modified |
| | | | oxo-7- | caelestis | Cg codon |
| | | | (phosphooxy)heptanoate | | usage |
| | | | synthase | | |
| Cg3A4BAC_13 | | A0A0B5ICI3 | 2-amino-4,5-dihydroxy-6- | Streptomyces | modified |
| | | | oxo-7- | vietnamensis | Cg codon |
| | | | (phosphooxy)heptanoate | | usage |
| | | | synthase | | |
| Cg3A4BAC_14 | 2287 | K0KAK8 | 2-amino-4,5-dihydroxy-6- | Saccharothrix | modified |
| | | | oxo-7- | espanaensis | Cg codon |
| | | | (phosphooxy)heptanoate | ATCC 51144 | usage |
| | | | synthase | | |
| Cg3A4BAC_15 | | K0JYX4 | 2-amino-4,5-dihydroxy-6- | Saccharothrix | modified |
| | | | oxo-7- | espanaensis | Cg codon |
| | | | (phosphooxy)heptanoate | ATCC 51144 | usage |
| | | | synthase | | |

| | | | | E2 Codon |
|---|---|---|---|---|
| | E2 | Enzyme 2 - | Enzyme 2 - | Optimization |
| Strain name | Uniprot ID | activity name | source organism | Abbrev. |
| Cg3A4BAC_01 | A0A166CII6 | 3-amino-4-benzoic acid | Methanobrevibacter | modified |
| | | synthase | curvatus | Cg codon |
| | | | | usage |
| Cg3A4BAC_02 | A0A0N9Z3W2 | 3-amino-4-benzoic acid | Thaumarchaeota | modified |
| | | synthase | archaeon MY3 | Cg codon |
| | | | | usage |
| Cg3A4BAC_04 | L0N4Y5 | 3-amino-4-benzoic acid | Streptomyces | modified |
| | | synthase | sp. WK-5344 | Cg codon |
| | | | | usage |
| Cg3A4BAC_05 | A0A0K2YDP9 | 3-amino-4-benzoic acid | Rhodococcus | modified |
| | | synthase | sp. RD6.2 | Cg codon |
| | | | | usage |
| Cg3A4BAC_06 | D6RTB7 | 3-amino-4-benzoic acid | Streptomyces | modified |
| | | synthase | murayamaensis | Cg codon |
| | | | | usage |
| Cg3A4BAC_07 | A0A088DA72 | 3-amino-4-benzoic acid | Streptomyces | modified |
| | | synthase | aureus | Cg codon |
| | | | | usage |
| Cg3A4BAC_08 | W5WBR4 | 3-amino-4-benzoic acid | Kutzneria albida | modified |
| | | synthase | DSM 43870 | Cg codon |
| | | | | usage |
| Cg3A4BAC_09 | K0JXI9 | 3-amino-4-benzoic acid | Saccharothrix | modified |
| | | synthase | espanaensis | Cg codon |
| | | | ATCC 51144 | usage |
| Cg3A4BAC_10 | K0K7Z4 | 3-amino-4-benzoic acid | Saccharothrix | modified |
| | | synthase | espanaensis | Cg codon |
| | | | ATCC 51144 | usage |
| Cg3A4BAC_11 | A0A0D8I0B7 | 3-amino-4-benzoic acid | Rhodococcus | modified |
| | | synthase | sp. AD45 | Cg codon |
| | | | | usage |
| Cg3A4BAC_12 | A0A117ECS3 | 3-amino-4-benzoic acid | Streptomyces | modified |
| | | synthase | scabiei | Cg codon |
| | | | | usage |
| Cg3A4BAC_13 | F3NEM9 | 3-amino-4-benzoic acid | Streptomyces | modified |
| | | synthase | griseoaurantiacus | Cg codon |
| | | | M045 | usage |
| Cg3A4BAC_14 | A0A0K6LJE3 | 3-amino-4-benzoic acid | Bacillus cereus | modified |
| | | synthase | | Cg codon |
| | | | | usage |
| Cg3A4BAC_15 | A0A088UAL1 | 3-amino-4-benzoic acid | Burkholderia | modified |
| | | synthase | cepacia | Cg codon |
| | | | ATCC 25416 | usage |

TABLE 2
First-round genetic engineering in Saccharomyces cerevisiae

| | | | | | E1 Codon |
|---|---|---|---|---|---|
| | Titer | E1 | Enzyme 1 - | Enzyme 1 - | Optimization |
| Strain name | (μg/L) | Uniprot ID | activity name | source organism | Abbrev. |
| Sc3A4BAC_01 | 0 | A0A166BTP9 | 2-amino-4,5-dihydroxy-6- | Methanobrevibacter | modified |
| | | | oxo-7- | oralis | Cg codon |
| | | | (phosphooxy)heptanoate | | usage |
| | | | synthase | | |
| Sc3A4BAC_02 | 0 | A0A166CVJ4 | 2-amino-4,5-dihydroxy-6- | Methanobrevibacter | modified |
| | | | oxo-7- | cuticularis | Cg codon |
| | | | (phosphooxy)heptanoate | | usage |
| | | | synthase | | |
| Sc3A4BAC_04 | 0 | A0A166D2B9 | 2-amino-4,5-dihydroxy-6- | Methanobrevibacter | modified |
| | | | oxo-7- | filiformis | Cg codon |
| | | | (phosphooxy)heptanoate | | usage |
| | | | synthase | | |
| Sc3A4BAC_05 | | A0JC77 | 2-amino-4,5-dihydroxy-6- | Streptomyces | modified |
| | | | oxo-7- | griseus | Cg codon |
| | | | (phosphooxy)heptanoate | | usage |
| | | | synthase | | |
| Sc3A4BAC_06 | 0 | A0A0N8G7L8 | 2-amino-4,5-dihydroxy-6- | Streptomyces | modified |
| | | | oxo-7- | anulatus | Cg codon |
| | | | (phosphooxy)heptanoate | | usage |
| | | | synthase | | |
| Sc3A4BAC_07 | 0 | A0A0S3TVT4 | 2-amino-4,5-dihydroxy-6- | Streptomyces | modified |
| | | | oxo-7- | cremeus | Cg codon |
| | | | (phosphooxy)heptanoate | | usage |
| | | | synthase | | |
| Sc3A4BAC_08 | | A0A0M4DD67 | 2-amino-4,5-dihydroxy-6- | Streptomyces | modified |
| | | | oxo-7- | pristinaespiralis | Cg codon |
| | | | (phosphooxy)heptanoate | | usage |
| | | | synthase | | |
| Sc3A4BAC_09 | 0 | A0A0Q8F363 | 2-amino-4,5-dihydroxy-6- | Streptomyces | modified |
| | | | oxo-7- | sp. Root63 | Cg codon |
| | | | (phosphooxy)heptanoate | | usage |
| | | | synthase | | |
| Sc3A4BAC_10 | | A0A0P6UHB3 | 2-amino-4,5-dihydroxy-6- | Streptomyces | modified |
| | | | oxo-7- | anulatus | Cg codon |
| | | | (phosphooxy)heptanoate | | usage |
| | | | synthase | | |
| Sc3A4BAC_11 | 0 | W5W353 | 2-amino-4,5-dihydroxy-6- | Kutzneria albida | modified |
| | | | oxo-7- | DSM 43870 | Cg codon |
| | | | (phosphooxy)heptanoate | | usage |
| | | | synthase | | |
| Sc3A4BAC_12 | 0 | A0A0M9XA57 | 2-amino-4,5-dihydroxy-6- | Streptomyces | modified |
| | | | oxo-7- | caelestis | Cg codon |
| | | | (phosphooxy)heptanoate | | usage |
| | | | synthase | | |
| Sc3A4BAC_13 | 0 | A0A0B5ICI3 | 2-amino-4,5-dihydroxy-6- | Streptomyces | modified |
| | | | oxo-7- | vietnamensis | Cg codon |
| | | | (phosphooxy)heptanoate | | usage |
| | | | synthase | | |
| Sc3A4BAC_14 | 0 | K0KAK8 | 2-amino-4,5-dihydroxy-6- | Saccharothrix | modified |
| | | | oxo-7- | espanaensis | Cg codon |
| | | | (phosphooxy)heptanoate | ATCC 51144 | usage |
| | | | synthase | | |
| Sc3A4BAC_15 | 0 | K0JYX4 | 2-amino-4,5-dihydroxy-6- | Saccharothrix | modified |
| | | | oxo-7- | espanaensis | Cg codon |
| | | | (phosphooxy)heptanoate | ATCC 51144 | usage |
| | | | synthase | | |
| Sc3A4BAC_16 | 0 | A0A143C222 | 2-amino-4,5-dihydroxy-6- | Streptomyces | modified |
| | | | oxo-7- | sp. S10(2016) | Cg codon |
| | | | (phosphooxy)heptanoate | | usage |
| | | | synthase | | |

| | | | | E2 Codon |
|---|---|---|---|---|
| | E2 | Enzyme 2 - | Enzyme 2 - | Optimization |
| Strain name | Uniprot ID | activity name | source organism | Abbrev. |
| Sc3A4BAC_01 | A0A166CII6 | 3-amino-4-benzoic acid | Methanobrevibacter | modified |
| | | synthase | curvatus | Cg codon |
| | | | | usage |
| Sc3A4BAC_02 | A0A0N9Z3W2 | 3-amino-4-benzoic acid | Thaumarchaeota | modified |
| | | synthase | archaeon MY3 | Cg codon |
| | | | | usage |
| Sc3A4BAC_04 | L0N4Y5 | 3-amino-4-benzoic acid | Streptomyces | modified |
| | | synthase | sp. WK-5344 | Cg codon |
| | | | | usage |
| Sc3A4BAC_05 | A0A0K2YDP9 | 3-amino-4-benzoic acid | Rhodococcus | modified |
| | | synthase | sp. RD6.2 | Cg codon |
| | | | | usage |
| Sc3A4BAC_06 | D6RTB7 | 3-amino-4-benzoic acid | Streptomyces | modified |
| | | synthase | murayamaensis | Cg codon |
| | | | | usage |
| Sc3A4BAC_07 | A0A088DA72 | 3-amino-4-benzoic acid | Streptomyces | modified |
| | | synthase | aureus | Cg codon |
| | | | | usage |
| Sc3A4BAC_08 | W5WBR4 | 3-amino-4-benzoic acid | Kutzneria albida | modified |
| | | synthase | DSM 43870 | Cg codon |
| | | | | usage |
| Sc3A4BAC_09 | K0JXI9 | 3-amino-4-benzoic acid | Saccharothrix | modified |
| | | synthase | espanaensis | Cg codon |
| | | | ATCC 51144 | usage |
| Sc3A4BAC_10 | K0K7Z4 | 3-amino-4-benzoic acid | Saccharothrix | modified |
| | | synthase | espanaensis | Cg codon |
| | | | ATCC 51144 | usage |
| Sc3A4BAC_11 | A0A0D8I0B7 | 3-amino-4-benzoic acid | Rhodococcus | modified |
| | | synthase | sp. AD45 | Cg codon |
| | | | | usage |
| Sc3A4BAC_12 | A0A117ECS3 | 3-amino-4-benzoic acid | Streptomyces | modified |
| | | synthase | scabiei | Cg codon |
| | | | | usage |
| Sc3A4BAC_13 | F3NEM9 | 3-amino-4-benzoic acid | Streptomyces | modified |
| | | synthase | griseoaurantiacus | Cg codon |
| | | | M045 | usage |
| Sc3A4BAC_14 | A0A0K6LJE3 | 3-amino-4-benzoic acid | Bacillus cereus | modified |
| | | synthase | | Cg codon |
| | | | | usage |
| Sc3A4BAC_15 | A0A088UAL1 | 3-amino-4-benzoic acid | Burkholderia | modified |
| | | synthase | cepacia | Cg codon |
| | | | ATCC 25416 | usage |
| Sc3A4BAC_16 | A0A165U0M8 | 3-amino-4-benzoic acid | Pseudovibrio | modified |
| | | synthase | axinellae | Cg codon |
| | | | | usage |

TABLE 3
Second-round genetic engineering in Corynebacteria glutamicum
Corynebacteria glutamicum strains expressed enzymes as indicated in the table below. In addition to the
genetic changes in the table below, the strains also contained the best enzymes from first round:
2-amino-4,5-dihydroxy-6-oxo-7-(phosphooxy)heptanoate synthase (UniProt ID A0A0Q8F363) from Streptomyces sp.
Root63, and 3-amino-4-benzoic acid synthase (UniProt ID K0JXI9) from Saccharothrix espanaensis ATCC 51144.

| | | | | E1 | | E1 Codon |
|---|---|---|---|---|---|---|
| | Titer | E1 | Enzyme 1 - | Modifi- | Enzyme 1 - | Optimization |
| Strain name | (μg/L) | Uniprot ID | activity name | cations | source organism | Abbrev. |
| Cg3A4BAC_09 | 748.52 | A0A0Q8F363 | 2-amino-4,5-dihydroxy- | | Streptomyces | modified |
| | | | 6-oxo-7- | | sp. Root63 | Corynebacterium |
| | | | (phosphooxy)heptanoate | | | glutamicum |
| | | | synthase | | | codon usage |
| Cg3A4BAC_20 | 777.14 | P10869 | Aspartate kinase | G452D | Saccharomyces | modified codon |
| | | | | | cerevisiae | usage for Cg |
| | | | | | S288c | and Sc |
| Cg3A4BAC_21 | 1208.52 | K0JXI9 | 3-amino-4-benzoic acid | | Saccharothrix | modified codon |
| | | | synthase | | espanaensis | usage for Cg |
| | | | | | ATCC 51144 | and Sc |
| Cg3A4BAC_22 | 609.29 | A0JC77 | 2-amino-4,5-dihydroxy- | | Streptomyces | modified |
| | | | 6-oxo-7- | | griseus | Corynebacterium |
| | | | (phosphooxy)heptanoate | | | glutamicum |
| | | | synthase | | | codon usage |
| Cg3A4BAC_23 | 697.83 | P26512 | aspartate kinase activity | S317A | Corynebacterium | Corynebacterium |
| | | | | | glutamicum | glutamicum |
| Cg3A4BAC_24 | 670.61 | P08660 | Aspartokinase III | E250K | Escherichia coli | modified codon |
| | | | | | (strain K12) | usage for Cg |
| | | | | | | and Sc |
| Cg3A4BAC_25 | | P08660 | Aspartokinase III | T344M | Escherichia coli | modified codon |
| | | | | | (strain K12) | usage for Cg |
| | | | | | | and Sc |
| Cg3A4BAC_26 | 707.05 | P08660 | Aspartokinase III | T352I | Escherichia coli | modified codon |
| | | | | | (strain K12) | usage for Cg |
| | | | | | | and Sc |
| Cg3A4BAC_27 | | P08660 | Aspartokinase III | M318I | Escherichia coli | modified codon |
| | | | | | (strain K12) | usage for Cg |
| | | | | | | and Sc |
| Cg3A4BAC_28 | 453.86 | P08660 | Aspartokinase III | G323D | Escherichia coli | modified codon |
| | | | | | (strain K12) | usage for Cg |
| | | | | | | and Sc |
| Cg3A4BAC_29 | 659.59 | P08660 | Aspartokinase III | L325F | Escherichia coli | modified codon |
| | | | | | (strain K12) | usage for Cg |
| | | | | | | and Sc |
| Cg3A4BAC_30 | | P08660 | Aspartokinase III | S345L | Escherichia coli | modified codon |
| | | | | | (strain K12) | usage for Cg |
| | | | | | | and Sc |
| Cg3A4BAC_31 | | P26512 | Aspartokinase | Q298G | Corynebacterium | modified codon |
| | | | | | glutamicum | usage for Cg |
| | | | | | ATCC 13032 | and Sc |
| Cg3A4BAC_32 | 593.47 | P26512 | Aspartokinase | S301Y | Corynebacterium | modified codon |
| | | | | | glutamicum | usage for Cg |
| | | | | | ATCC 13032 | and Sc |
| Cg3A4BAC_33 | 808.57 | P32801 | Aspartate- | | Saccharomyces | modified codon |
| | | | semialdehyde | | cerevisiae | usage for Cg |
| | | | dehydrogenase (ASA | | S288c | and Sc |
| | | | dehydrogenase) | | | |
| | | | (ASADH) | | | |
| Cg3A4BAC_34 | 549.93 | P0C1D8 | Aspartate- | | Corynebacterium | modified codon |
| | | | semialdehyde | | glutamicum | usage for Cg |
| | | | dehydrogenase (ASA | | ATCC 13032 | and Sc |
| | | | dehydrogenase) | | | |
| | | | (ASADH) | | | |
| Cg3A4BAC_35 | 536.42 | P0C1D8 | Aspartate- | | Corynebacterium | modified codon |
| | | | semialdehyde | | glutamicum | usage for Cg |
| | | | dehydrogenase (ASA | | ATCC 13032 | and Sc |
| | | | dehydrogenase) | | | |
| | | | (ASADH) | | | |
| Cg3A4BAC_36 | 646.29 | P0C1D8 | Aspartate- | | Corynebacterium | modified codon |
| | | | semialdehyde | | glutamicum | usage for Cg |
| | | | dehydrogenase (ASA | | ATCC 13032 | and Sc |
| | | | dehydrogenase) | | | |
| | | | (ASADH) | | | |
| Cg3A4BAC_37 | 745.91 | P0C1D8 | aspartate | D66G, | Corynebacterium | native |
| | | | semialdehyde | S202F, | glutamicum | |
| | | | dehydrogenase | R234H, | ATCC 13032 | |
| | | | | D272E, | | |
| | | | | K285E | | |
| Cg3A4BAC_38 | 555.06 | P0C1D8 | aspartate | D66G, | Corynebacterium | native |
| | | | semialdehyde | S202F, | glutamicum | |
| | | | dehydrogenase | R234H, | ATCC 13032 | |
| | | | | D272E, | | |
| | | | | K285E | | |
| Cg3A4BAC_39 | 612.52 | P0C1D8 | aspartate | D66G, | Corynebacterium | native |
| | | | semialdehyde | S202F, | glutamicum | |
| | | | dehydrogenase | R234H, | ATCC 13032 | |
| | | | | D272E, | | |
| | | | | K285E | | |
| Cg3A4BAC_40 | | P32801 | Aspartate- | | Saccharomyces | modified codon |
| | | | semialdehyde | | cerevisiae | usage for Cg |
| | | | dehydrogenase (ASA | | S288c | and Sc |
| | | | dehydrogenase) | | | |
| | | | (ASADH) | | | |
| Cg3A4BAC_41 | | P0C1D8 | aspartate | D66G, | Corynebacterium | modified codon |
| | | | semialdehyde | S202F, | glutamicum | usage for Cg |
| | | | dehydrogenase | R234H, | ATCC 13032 | and Sc |
| | | | | D272E, | | |
| | | | | K285E | | |
| Cg3A4BAC_42 | 779.83 | A0A0B5ICI3 | 2-amino-4,5-dihydroxy- | | Streptomyces | modified |
| | | | 6-oxo-7- | | vietnamensis | Corynebacterium |
| | | | (phosphooxy)heptanoate | | | glutamicum |
| | | | synthase | | | codon usage |
| Cg3A4BAC_43 | 401.42 | Q12128 | Malate dehydrogenase | | Saccharomyces | modified codon |
| | | | | | cerevisiae | usage for Cg |
| | | | | | S288c | and Sc |
| Cg3A4BAC_44 | 668.17 | Q8NTR2 | Aspartate transaminase | | Corynebacterium | modified codon |
| | | | | | glutamicum | usage for Cg |
| | | | | | ATCC 13032 | and Sc |
| Cg3A4BAC_45 | 581.39 | A0A0M4DD67 | 2-amino-4,5-dihydroxy- | | Streptomyces | modified |
| | | | 6-oxo-7- | | pristinaespiralis | Corynebacterium |
| | | | (phosphooxy)heptanoate | | | glutamicum |
| | | | synthase | | | codon usage |
| Cg3A4BAC_46 | 309.68 | P0C1D8 | aspartate | D66G, | Corynebacterium | native |
| | | | semialdehyde | S202F, | glutamicum | |
| | | | dehydrogenase | R234H, | ATCC 13032 | |
| | | | | D272E, | | |
| | | | | K285E | | |
| Cg3A4BAC_47 | 743.65 | Q01802 | Aspartate transaminase | | Saccharomyces | modified codon |
| | | | | | cerevisiae | usage for Cg |
| | | | | | S288c | and Sc |
| Cg3A4BAC_48 | 3463.89 | W5WBR4 | 3-amino-4-benzoic acid | | Kutzneria albida | modified |
| | | | synthase | | DSM 43870 | Corynebacterium |
| | | | | | | glutamicum |
| | | | | | | codon usage |
| Cg3A4BAC_49 | 2199.52 | A0A0K2YDP9 | 3-amino-4-benzoic acid | | Rhodococcus | modified |
| | | | synthase | | sp. RD6.2 | Corynebacterium |
| | | | | | | glutamicum |
| | | | | | | codon usage |
| Cg3A4BAC_50 | 652.57 | P26512 | aspartate kinase | A279T, | Corynebacterium | native |
| | | | | S317A | glutamicum | |
| | | | | | ATCC 13032 | |
| Cg3A4BAC_51 | 553.56 | P23542 | aspartate | | Saccharomyces | modified codon |
| | | | aminotransferase | | cerevisiae | usage for Cg |
| | | | activity | | S288c | and Sc |
| Cg3A4BAC_52 | | N1P4U6 | aspartate kinase | | Saccharomyces | native |
| | | | | | cerevisiae | |
| | | | | | CEN.PK113-7D | |
| Cg3A4BAC_53 | 743.73 | P32801 | Aspartate- | | Saccharomyces | modified codon |
| | | | semialdehyde | | cerevisiae | usage for Cg |
| | | | dehydrogenase (ASA | | S288c | and Sc |
| | | | dehydrogenase) | | | |
| | | | (ASADH) | | | |
| Cg3A4BAC_54 | 444.11 | P0C1D8 | aspartate | D66G, | Corynebacterium | native |
| | | | semialdehyde | S202F, | glutamicum | |
| | | | dehydrogenase | R234H, | ATCC 13032 | |
| | | | | D272E, | | |
| | | | | K285E | | |
| Cg3A4BAC_55 | | P0C1D8 | Aspartate- | | Corynebacterium | modified codon |
| | | | semialdehyde | | glutamicum | usage for Cg |
| | | | dehydrogenase (ASA | | ATCC 13032 | and Sc |
| | | | dehydrogenase) | | | |
| | | | (ASADH) | | | |
| Cg3A4BAC_56 | 631.42 | Q8NN33 | Malate dehydrogenase | | Corynebacterium | modified codon |
| | | | | | glutamicum | usage for Cg |
| | | | | | ATCC 13032 | and Sc |
| Cg3A4BAC_57 | 597.2 | A0A0Q8F363 | 2-amino-4,5-dihydroxy- | | Streptomyces | modified |
| | | | 6-oxo-7- | | sp. Root63 | Corynebacterium |
| | | | (phosphooxy)heptanoate | | | glutamicum |
| | | | synthase | | | codon usage |
| Cg3A4BAC_58 | 528.86 | P26512 | aspartate kinase activity | S317A | Corynebacterium | Corynebacterium |
| | | | | | glutamicum | glutamicum |
| Cg3A4BAC_59 | | P0C1D8 | Aspartate- | | Corynebacterium | modified codon |
| | | | semialdehyde | | glutamicum | usage for Cg |
| | | | dehydrogenase (ASA | | ATCC 13032 | and Sc |
| | | | dehydrogenase) | | | |
| | | | (ASADH) | | | |
| Cg3A4BAC_60 | | W5W353 | 2-amino-4,5-dihydroxy- | | Kutzneria albida | modified |
| | | | 6-oxo-7- | | DSM 43870 | Corynebacterium |
| | | | (phosphooxy)heptanoate | | | glutamicum |
| | | | synthase | | | codon usage |
| Cg3A4BAC_61 | | P0C1D8 | Aspartate- | | Corynebacterium | modified codon |
| | | | semialdehyde | | glutamicum | usage for Cg |
| | | | dehydrogenase (ASA | | ATCC 13032 | and Sc |
| | | | dehydrogenase) | | | |
| | | | (ASADH) | | | |

| | | | | E2 Codon |
|---|---|---|---|---|
| | E2 | Enzyme 2 - | Enzyme 2 - | Optimization |
| Strain name | Uniprot ID | activity name | source organism | Abbrev. |
| Cg3A4BAC_09 | K0JXI9 | 3-amino-4-benzoic acid | Saccharothrix | modified |
| | | synthase | espanaensis | Corynebacterium |
| | | | ATCC 51144 | glutamicum |
| | | | | codon usage |
| Cg3A4BAC_20 | | | | |
| Cg3A4BAC_21 | | | | |
| Cg3A4BAC_22 | | | | |
| Cg3A4BAC_23 | | | | |
| Cg3A4BAC_24 | | | | |
| Cg3A4BAC_25 | | | | |
| Cg3A4BAC_26 | | | | |
| Cg3A4BAC_27 | | | | |
| Cg3A4BAC_28 | | | | |
| Cg3A4BAC_29 | | | | |
| Cg3A4BAC_30 | | | | |
| Cg3A4BAC_31 | | | | |
| Cg3A4BAC_32 | | | | |
| Cg3A4BAC_33 | | | | |
| Cg3A4BAC_34 | | | | |
| Cg3A4BAC_35 | Q8NN33 | Malate dehydrogenase | Corynebacterium | modified codon |
| | | | glutamicum | usage for Cg |
| | | | ATCC 13032 | and Sc |
| Cg3A4BAC_36 | Q8NN33 | Malate dehydrogenase | Corynebacterium | modified codon |
| | | | glutamicum | usage for Cg |
| | | | ATCC 13032 | and Sc |
| Cg3A4BAC_37 | Q8NTR2 | Aspartate transaminase | Corynebacterium | modified codon |
| | | | glutamicum | usage for Cg |
| | | | ATCC 13032 | and Sc |
| Cg3A4BAC_38 | Q8NN33 | Malate dehydrogenase | Corynebacterium | modified codon |
| | | | glutamicum | usage for Cg |
| | | | ATCC 13032 | and Sc |
| Cg3A4BAC_39 | Q8NTR2 | Aspartate transaminase | Corynebacterium | modified codon |
| | | | glutamicum | usage for Cg |
| | | | ATCC 13032 | and Sc |
| Cg3A4BAC_40 | P23542 | aspartate | Saccharomyces | modified codon |
| | | aminotransferase | cerevisiae | usage for Cg |
| | | activity | S288c | and Sc |
| Cg3A4BAC_41 | Q8NN33 | Malate dehydrogenase | Corynebacterium | modified codon |
| | | | glutamicum | usage for Cg |
| | | | ATCC 13032 | and Sc |
| Cg3A4BAC_42 | | | | |
| Cg3A4BAC_43 | | | | |
| Cg3A4BAC_44 | | | | |
| Cg3A4BAC_45 | | | | |
| Cg3A4BAC_46 | | | | |
| Cg3A4BAC_47 | | | | |
| Cg3A4BAC_48 | | | | |
| Cg3A4BAC_49 | | | | |
| Cg3A4BAC_50 | | | | |
| Cg3A4BAC_51 | | | | |
| Cg3A4BAC_52 | | | | |
| Cg3A4BAC_53 | | | | |
| Cg3A4BAC_54 | | | | |
| Cg3A4BAC_55 | | | | |
| Cg3A4BAC_56 | | | | |
| Cg3A4BAC_57 | | | | |
| Cg3A4BAC_58 | | | | |
| Cg3A4BAC_59 | | | | |
| Cg3A4BAC_60 | | | | |
| Cg3A4BAC_61 | Q8NTR2 | Aspartate transaminase | Corynebacterium | modified codon |
| | | | glutamicum | usage for Cg |
| | | | ATCC 13032 | and Sc |

TABLE 4
Host evaluation - first-round genetic engineering in Yarrowia lipolytica
Yarrowia lipolytica expressed enzymes from Host Evaluation round for production of 3-amino-4-hydroxybenzoic acid.

| | | | | | E1 Codon | | |
|---|---|---|---|---|---|---|---|
| | Titer | E1 | Enzyme 1 - | Enzyme 1 - | Optimization | E2 | Enzyme 2 - |
| Strain name | (μg/L) | Uniprot ID | activity name | source organism | Abbrev. | Uniprot ID | activity name |
| YI3A4BAC_01 | 0 | K0JXI9 | 3-amino-4- | Saccharothrix | Bacillus | K0KAK8 | 2-amino-4,5- |
| | | | hydroxybenzoic | espanaensis | subtillus | | dihydroxy-6-one- |
| | | | acid synthase | ATCC 51144 | | | heptanoic acid- |
| | | | | | | | 7-phosphate |
| | | | | | | | synthase |
| YI3A4BAC_02 | 0 | K0JXI9 | 3-amino-4- | Saccharothrix | Saccharomyces | K0KAK8 | 2-amino-4,5- |
| | | | hydroxybenzoic | espanaensis | cerevisiae | | dihydroxy-6-one- |
| | | | acid synthase | ATCC 51144 | | | heptanoic acid- |
| | | | | | | | 7-phosphate |
| | | | | | | | synthase |
| YI3A4BAC_03 | 0 | A0A1T3V8D3 | 3- | Bacillus | Bacillus | NCBI- | 2-amino-4,5- |
| | | | dehydroquinate | anthracis | subtillus | CUB39904.1 | dihydroxy-6-one- |
| | | | synthase | | | | heptanoic acid- |
| | | | | | | | 7-phosphate |
| | | | | | | | synthase |
| YI3A4BAC_04 | 0 | A0A1T3V8D3 | 3- | Bacillus | Saccharomyces | NCBI- | 2-amino-4,5- |
| | | | dehydroquinate | anthracis | cerevisiae | CUB39904.1 | dihydroxy-6-one- |
| | | | synthase | | | | heptanoic acid- |
| | | | | | | | 7-phosphate |
| | | | | | | | synthase |
| YI3A4BAC_05 | 0 | A0A1T3V8D3 | 3- | Bacillus | Yarrowia | NCBI- | 2-amino-4,5- |
| | | | dehydroquinate | anthracis | lipolytica | CUB39904.1 | dihydroxy-6-one- |
| | | | synthase | | | | heptanoic acid- |
| | | | | | | | 7-phosphate |
| | | | | | | | synthase |
| YI3A4BAC_06 | 0 | K0JXI9 | 3-amino-4- | Saccharothrix | Bacillus | A0A0Q8F363 | 2-amino-4,5- |
| | | | hydroxybenzoic | espanaensis | subtillus | | dihydroxy-6-one- |
| | | | acid synthase | ATCC 51144 | | | heptanoic acid- |
| | | | | | | | 7-phosphate |
| | | | | | | | synthase |
| YI3A4BAC_07 | 0 | K0JXI9 | 3-amino-4- | Saccharothrix | Saccharomyces | A0A0Q8F363 | 2-amino-4,5- |
| | | | hydroxybenzoic | espanaensis | cerevisiae | | dihydroxy-6-one- |
| | | | acid synthase | ATCC 51144 | | | heptanoic acid- |
| | | | | | | | 7-phosphate |
| | | | | | | | synthase |
| YI3A4BAC_08 | 0 | K0JXI9 | 3-amino-4- | Saccharothrix | Yarrowia | A0A0Q8F363 | 2-amino-4,5- |
| | | | hydroxybenzoic | espanaensis | lipolytica | | dihydroxy-6-one- |
| | | | acid synthase | ATCC 51144 | | | heptanoic acid- |
| | | | | | | | 7-phosphate |
| | | | | | | | synthase |
| YI3A4BAC_09 | 0 | K0JXI9 | 3-amino-4- | Saccharothrix | Saccharomyces | K0KAK8 | 2-amino-4,5- |
| | | | hydroxybenzoic | espanaensis | cerevisiae | | dihydroxy-6-one- |
| | | | acid synthase | ATCC 51144 | | | heptanoic acid- |
| | | | | | | | 7-phosphate |
| | | | | | | | synthase |
| YI3A4BAC_10 | 0 | K0JXI9 | 3-amino-4- | Saccharothrix | Yarrowia | K0KAK8 | 2-amino-4,5- |
| | | | hydroxybenzoic | espanaensis | lipolytica | | dihydroxy-6-one- |
| | | | acid synthase | ATCC 51144 | | | heptanoic acid- |
| | | | | | | | 7-phosphate |
| | | | | | | | synthase |
| YI3A4BAC_11 | 0 | A0A1T3V8D3 | 3- | Bacillus | modified codon | NCBI- | 2-amino-4,5- |
| | | | dehydroquinate | anthracis | usage for Cg | CUB39904.1 | dihydroxy-6-one- |
| | | | synthase | | and Sc | | heptanoic acid- |
| | | | | | | | 7-phosphate |
| | | | | | | | synthase |
| YI3A4BAC_12 | 0 | A0A1T3V8D3 | 3- | Bacillus | Saccharomyces | NCBI- | 2-amino-4,5- |
| | | | dehydroquinate | anthracis | cerevisiae | CUB39904.1 | dihydroxy-6-one- |
| | | | synthase | | | | heptanoic acid- |
| | | | | | | | 7-phosphate |
| | | | | | | | synthase |
| YI3A4BAC_13 | 0 | A0A1T3V8D3 | 3- | Bacillus | Yarrowia | NCBI- | 2-amino-4,5- |
| | | | dehydroquinate | anthracis | lipolytica | CUB39904.1 | dihydroxy-6-one- |
| | | | synthase | | | | heptanoic acid- |
| | | | | | | | 7-phosphate |
| | | | | | | | synthase |
| YI3A4BAC_14 | 0 | K0JXI9 | 3-amino-4- | Saccharothrix | Saccharomyces | A0A0Q8F363 | 2-amino-4,5- |
| | | | hydroxybenzoic | espanaensis | cerevisiae | | dihydroxy-6-one- |
| | | | acid synthase | ATCC 51144 | | | heptanoic acid- |
| | | | | | | | 7-phosphate |
| | | | | | | | synthase |
| YI3A4BAC_15 | 0 | K0JXI9 | 3-amino-4- | Saccharothrix | Yarrowia | A0A0Q8F363 | 2-amino-4,5- |
| | | | hydroxybenzoic | espanaensis | lipolytica | | dihydroxy-6-one- |
| | | | acid synthase | ATCC 51144 | | | heptanoic acid- |
| | | | | | | | 7-phosphate |
| | | | | | | | synthase |
| YI3A4BAC_16 | 0 | A0A0Q8F363 | 2-amino-4,5- | Streptomyces | modified | K0JXI9 | 2-amino-4,5- |
| | | | dihydroxy-6- | sp. Root63 | Corynebacterium | | dihydroxy-6-one- |
| | | | oxo-7- | | glutamicum | | heptanoic acid- |
| | | | (phosphooxy) | | codon usage | | 7-phosphate |
| | | | heptanoate | | | | synthase |
| | | | synthase | | | | |

| | | E2 Codon | | | E3 | | E3 Codon |
|---|---|---|---|---|---|---|---|
| | Enzyme 2 - | Optimization | E3 | Enzyme 3 - | Modifi- | Enzyme 3 - | Optimization |
| Strain name | source organism | Abbrev. | Uniprot ID | activity name | cations | source organism | Abbrev. |
| YI3A4BAC_01 | Saccharothrix | Bacillus | P26512 | Aspartokinase | Q298G | Corynbacterium | Bacillus |
| | espanaensis | subtillus | | | | glutamicum | subtillus |
| | ATCC 51144 | | | | | ATCC 13032 | |
| YI3A4BAC_02 | Saccharothrix | Saccharomyces | P26512 | Aspartokinase | Q298G | Corynbacterium | Saccharomyces |
| | espanaensis | cerevisiae | | | | glutamicum | cerevisiae |
| | ATCC 51144 | | | | | ATCC 13032 | |
| YI3A4BAC_03 | Bacillus | Bacillus | P26512 | Aspartokinase | Q298G | Corynbacterium | Bacillus |
| | cereus | subtillus | | | | glutamicum | subtillus |
| | | | | | | ATCC 13032 | |
| YI3A4BAC_04 | Bacillus | Saccharomyces | P26512 | Aspartokinase | Q298G | Corynbacterium | Saccharomyces |
| | cereus | cerevisiae | | | | glutamicum | cerevisiae |
| | | | | | | ATCC 13032 | |
| YI3A4BAC_05 | Bacillus | Yarrowia | P26512 | Aspartokinase | Q298G | Corynbacterium | Yarrowia |
| | cereus | lipolytica | | | | glutamicum | lipolytica |
| | | | | | | ATCC 13032 | |
| YI3A4BAC_06 | Streptomyces | Bacillus | P26512 | Aspartokinase | Q298G | Corynbacterium | Bacillus |
| | sp. Root63 | subtillus | | | | glutamicum | subtillus |
| | | | | | | ATCC 13032 | |
| YI3A4BAC_07 | Streptomyces | Saccharomyces | P26512 | Aspartokinase | Q298G | Corynbacterium | Saccharomyces |
| | sp. Root63 | cerevisiae | | | | glutamicum | cerevisiae |
| | | | | | | ATCC 13032 | |
| YI3A4BAC_08 | Streptomyces | Yarrowia | P26512 | Aspartokinase | Q298G | Corynbacterium | Yarrowia |
| | sp. Root63 | lipolytica | | | | glutamicum | lipolytica |
| | | | | | | ATCC 13032 | |
| YI3A4BAC_09 | Saccharothrix | Saccharomyces | P26512 | Aspartokinase | S301Y | Corynbacterium | Saccharomyces |
| | espanaensis | cerevisiae | | | | glutamicum | cerevisiae |
| | ATCC 51144 | | | | | ATCC 13032 | |
| YI3A4BAC_10 | Saccharothrix | Yarrowia | P26512 | Aspartokinase | S301Y | Corynbacterium | Yarrowia |
| | espanaensis | lipolytica | | | | glutamicum | lipolytica |
| | ATCC 51144 | | | | | ATCC 13032 | |
| YI3A4BAC_11 | Bacillus | modified codon | P26512 | Aspartokinase | S301Y | Corynbacterium | modified codon |
| | cereus | usage for Cg | | | | glutamicum | usage for Cg |
| | | and Sc | | | | ATCC 13032 | and Sc |
| YI3A4BAC_12 | Bacillus | Saccharomyces | P26512 | Aspartokinase | S301Y | Corynbacterium | Saccharomyces |
| | cereus | cerevisiae | | | | glutamicum | cerevisiae |
| | | | | | | ATCC 13032 | |
| YI3A4BAC_13 | Bacillus | Yarrowia | P26512 | Aspartokinase | S301Y | Corynbacterium | Yarrowia |
| | cereus | lipolytica | | | | glutamicum | lipolytica |
| | | | | | | ATCC 13032 | |
| YI3A4BAC_14 | Streptomyces | Saccharomyces | P26512 | Aspartokinase | S301Y | Corynbacterium | Saccharomyces |
| | sp. Root63 | cerevisiae | | | | glutamicum | cerevisiae |
| | | | | | | ATCC 13032 | |
| YI3A4BAC_15 | Streptomyces | Yarrowia | P26512 | Aspartokinase | S301Y | Corynbacterium | Yarrowia |
| | sp. Root63 | lipolytica | | | | glutamicum | lipolytica |
| | | | | | | ATCC 13032 | |
| YI3A4BAC_16 | Saccharothrix | modified | | | | | |
| | espanaensis | Cg codon | | | | | |
| | ATCC 51144 | usage | | | | | |

TABLE 5
Host evaluation - first-round genetic engineering in Bacillus subtillus
Bacillus subtillus expressed enzymes from Host Evaluation round for production of 3-amino-4-hydroxybenzoic acid.

| | | | | | E1 Codon | | | E2 |
|---|---|---|---|---|---|---|---|---|
| | Titer | E1 | Enzyme 1 - | Enzyme 1 - | Optimization | E2 | Enzyme 2 - | Modifi- |
| Strain name | μg/L | Uniprot ID | activity name | source organism | Abbrev. | Uniprot ID | activity name | cations |
| Bs3A4BAC_02 | 0 | A0A0Q8F363 | 2-amino-4,5- | Streptomyces | modified | K0JXI9 | 3-amino- | |
| | | | dihydroxy-6- | sp. Root63 | Cg codon | | 4-benzoic | |
| | | | oxo-7-(phos- | | usage | | acid | |
| | | | phooxy)heptanoate | | | | synthase | |
| | | | synthase | | | | | |
| Bs3A4BAC_18 | 0 | A0A1T3V8D3 | 3-dehydro- | Bacillus | modified codon | NCBI- | No records | |
| | | | quinate | anthracis | usage for Cg | CUB39904.1 | were found. | |
| | | | synthase | | and Sc | | | |
| Bs3A4BAC_19 | 0 | A0A1T3V8D3 | 3-dehydro- | Bacillus | Saccharomyces | P26512 | Aspartokinase | S301Y |
| | | | quinate | anthracis | cerevisiae | | | |
| | | | synthase | | | | | |
| Bs3A4BAC_21 | 0 | K0JXI9 | 3-amino-4- | Saccharothrix | Saccharomyces | P26512 | Aspartokinase | S301Y |
| | | | hydroxyben- | espanaensis | cerevisiae | | | |
| | | | zoic acid | ATCC 51144 | | | | |
| | | | synthase | | | | | |

| | | E2 Codon | | | E3 | | E3 Codon |
|---|---|---|---|---|---|---|---|
| | Enzyme 2 - | Optimization | E3 | Enzyme 3 - | Modifi- | Enzyme 3 - | Optimization |
| Strain name | source organism | Abbrev. | Uniprot ID | activity name | cations | source organism | Abbrev. |
| Bs3A4BAC_02 | Saccharothrix | modified | P26512 | Aspartokinase | S301Y | Corynebacterium | Yarrowia |
| | espanaensis | Cg codon | | | | glutamicum | lipolytica |
| | (strain ATCC 51144/ | usage | | | | ATCC 13032 | |
| | DSM 44229/JCM 9112/ | | | | | | |
| | NBRC 15066/NRRL 15764) | | | | | | |
| Bs3A4BAC_18 | Bacillus | modified codon | | | | | |
| | cereus | usage for Cg | | | | | |
| | | and Sc | | | | | |
| Bs3A4BAC_19 | Corynebacterium | Saccharomyces | | | | | |
| | glutamicum | cerevisiae | | | | | |
| | (strain ATCC 13032/ | | | | | | |
| | DSM 20300/JCM 1318/ | | | | | | |
| | LMG 3730/NCIMB 10025) | | | | | | |
| Bs3A4BAC_21 | Corynebacterium | Saccharomyces | | | | | |
| | glutamicum | cerevisiae | | | | | |
| | (strain ATCC 13032/ | | | | | | |
| | DSM 20300/JCM 1318/ | | | | | | |
| | LMG 3730/NCIMB 10025) | | | | | | |

TABLE 6
Host evaluation - first-round genetic engineering in Saccharomyces cerevisiae
Saccharomyces cerevisiae expressed enzymes from Host Evaluation round for production of 3-amino-4-hydroxybenzoic acid.

| | | | | | E1 Codon | | |
|---|---|---|---|---|---|---|---|
| | Titer | E1 | Enzyme 1 - | Enzyme 1 - | Optimization | E2 | Enzyme 2 - |
| Strain name | (μg/L) | Uniprot ID | activity name | source organism | Abbrev. | Uniprot ID | activity name |
| Sc3A4BAC_80 | | K0JXI9 | 3-amino-4- | Saccharothrix | Bacillus | K0KAK8 | 2-amino-4,5- |
| | | | hydroxybenzoic | espanaensis | subtillus | | dihydroxy-6-one- |
| | | | acid synthase | ATCC 51144 | | | heptanoic acid-7- |
| | | | | | | | phosphate |
| | | | | | | | synthase |
| Sc3A4BAC_81 | | K0JXI9 | 3-amino-4- | Saccharothrix | Saccharomyces | K0KAK8 | 2-amino-4,5- |
| | | | hydroxybenzoic | espanaensis | cerevisiae | | dihydroxy-6-one- |
| | | | acid synthase | ATCC 51144 | | | heptanoic acid-7- |
| | | | | | | | phosphate |
| | | | | | | | synthase |
| Sc3A4BAC_82 | | K0JXI9 | 3-amino-4- | Saccharothrix | Yarrowia | K0KAK8 | 2-amino-4,5- |
| | | | hydroxybenzoic | espanaensis | lipolytica | | dihydroxy-6-one- |
| | | | acid synthase | ATCC 51144 | | | heptanoic acid-7- |
| | | | | | | | phosphate |
| | | | | | | | synthase |
| Sc3A4BAC_83 | | A0A1T3V8D3 | 3- | Bacillus | Bacillus | NCBI- | 2-amino-4,5- |
| | | | dehydroquinate | anthracis | subtillus | CUB39904.1 | dihydroxy-6-one- |
| | | | synthase | | | | heptanoic acid-7- |
| | | | | | | | phosphate |
| | | | | | | | synthase |
| Sc3A4BAC_84 | | A0A1T3V8D3 | 3- | Bacillus | modified codon | NCBI- | 2-amino-4,5- |
| | | | dehydroquinate | anthracis | usage for Cg | CUB39904.1 | dihydroxy-6-one- |
| | | | synthase | | and Sc | | heptanoic acid-7- |
| | | | | | | | phosphate |
| | | | | | | | synthase |
| Sc3A4BAC_85 | | A0A1T3V8D3 | 3- | Bacillus | Yarrowia | NCBI- | 2-amino-4,5- |
| | | | dehydroquinate | anthracis | lipolytica | CUB39904.1 | dihydroxy-6-one- |
| | | | synthase | | | | heptanoic acid-7- |
| | | | | | | | phosphate |
| | | | | | | | synthase |
| Sc3A4BAC_86 | | K0JXI9 | 3-amino-4- | Saccharothrix | Bacillus | A0A0Q8F363 | 2-amino-4,5- |
| | | | hydroxybenzoic | espanaensis | subtillus | | dihydroxy-6-one- |
| | | | acid synthase | ATCC 51144 | | | heptanoic acid-7- |
| | | | | | | | phosphate |
| | | | | | | | synthase |
| Sc3A4BAC_87 | | K0JXI9 | 3-amino-4- | Saccharothrix | Saccharomyces | A0A0Q8F363 | 2-amino-4,5- |
| | | | hydroxybenzoic | espanaensis | cerevisiae | | dihydroxy-6-one- |
| | | | acid synthase | ATCC 51144 | | | heptanoic acid-7- |
| | | | | | | | phosphate |
| | | | | | | | synthase |
| Sc3A4BAC_88 | | K0JXI9 | 3-amino-4- | Saccharothrix | Yarrowia | A0A0Q8F363 | 2-amino-4,5- |
| | | | hydroxybenzoic | espanaensis | lipolytica | | dihydroxy-6-one- |
| | | | acid synthase | ATCC 51144 | | | heptanoic acid-7- |
| | | | | | | | phosphate |
| | | | | | | | synthase |
| Sc3A4BAC_89 | | K0JXI9 | 3-amino-4- | Saccharothrix | Saccharomyces | K0KAK8 | 2-amino-4,5- |
| | | | hydroxybenzoic | espanaensis | cerevisiae | | dihydroxy-6-one- |
| | | | acid synthase | ATCC 51144 | | | heptanoic acid-7- |
| | | | | | | | phosphate |
| | | | | | | | synthase |
| Sc3A4BAC_90 | | A0A1T3V8D3 | 3- | Bacillus | Saccharomyces | NCBI- | 2-amino-4,5- |
| | | | dehydroquinate | anthracis | cerevisiae | CUB39904.1 | dihydroxy-6-one- |
| | | | synthase | | | | heptanoic acid-7- |
| | | | | | | | phosphate |
| | | | | | | | synthase |
| Sc3A4BAC_91 | | A0A1T3V8D3 | 3- | Bacillus | Yarrowia | NCBI- | 2-amino-4,5- |
| | | | dehydroquinate | anthracis | lipolytica | CUB39904.1 | dihydroxy-6-one- |
| | | | synthase | | | | heptanoic acid-7- |
| | | | | | | | phosphate |
| | | | | | | | synthase |
| Sc3A4BAC_92 | 4.908 | K0JXI9 | 3-amino-4- | Saccharothrix | Saccharomyces | A0A0Q8F363 | 2-amino-4,5- |
| | | | hydroxybenzoic | espanaensis | cerevisiae | | dihydroxy-6-one- |
| | | | acid synthase | ATCC 51144 | | | heptanoic acid-7- |
| | | | | | | | phosphate |
| | | | | | | | synthase |
| Sc3A4BAC_93 | 7.486 | K0JXI9 | 3-amino-4- | Saccharothrix | Yarrowia | A0A0Q8F363 | 2-amino-4,5- |
| | | | hydroxybenzoic | espanaensis | lipolytica | | dihydroxy-6-one- |
| | | | acid synthase | ATCC 51144 | | | heptanoic acid-7- |
| | | | | | | | phosphate |
| | | | | | | | synthase |
| Sc3A4BAC_94 | 23.675 | A0A0Q8F363 | 2-amino-4,5- | Streptomyces | modified | K0JXI9 | 3-amino-4-benzoic |
| | | | dihydroxy-6- | sp. Root63 | Cg codon | | acid synthase |
| | | | oxo-7- | | usage | | |
| | | | (phosphooxy) | | | | |
| | | | heptanoate | | | | |
| | | | synthase | | | | |

| | | E2 Codon | | | E3 | | E3 Codon |
|---|---|---|---|---|---|---|---|
| | Enzyme 2 - | Optimization | E3 | Enzyme 3 - | Modifi- | Enzyme 3 - | Optimization |
| Strain name | source organism | Abbrev. | Uniprot ID | activity name | cations | source organism | Abbrev. |
| Sc3A4BAC_80 | Saccharothrix | Bacillus | P26512 | Aspartokinase | Q298G | Corynebacterium | Bacillus |
| | espanaensis | subtillus | | | | glutamicum | subtillus |
| | ATCC 51144 | | | | | ATCC 13032 | |
| Sc3A4BAC_81 | Saccharothrix | Saccharomyces | P26512 | Aspartokinase | Q298G | Corynebacterium | Saccharomyces |
| | espanaensis | cerevisiae | | | | glutamicum | cerevisiae |
| | ATCC 51144 | | | | | ATCC 13032 | |
| Sc3A4BAC_82 | Saccharothrix | Yarrowia | P26512 | Aspartokinase | Q298G | Corynebacterium | Yarrowia |
| | espanaensis | lipolytica | | | | glutamicum | lipolytica |
| | ATCC 51144 | | | | | ATCC 13032 | |
| Sc3A4BAC_83 | Bacillus | Bacillus | P26512 | Aspartokinase | Q298G | Corynebacterium | Bacillus |
| | cereus | subtillus | | | | glutamicum | subtillus |
| | | | | | | ATCC 13032 | |
| Sc3A4BAC_84 | Bacillus | modified codon | P26512 | Aspartokinase | Q298G | Corynebacterium | modified codon |
| | cereus | usage for Cg | | | | glutamicum | usage for Cg |
| | | and Sc | | | | ATCC 13032 | and Sc |
| Sc3A4BAC_85 | Bacillus | Yarrowia | P26512 | Aspartokinase | Q298G | Corynebacterium | Yarrowia |
| | cereus | lipolytica | | | | glutamicum | lipolytica |
| | | | | | | ATCC 13032 | |
| Sc3A4BAC_86 | Streptomyces | Bacillus | P26512 | Aspartokinase | Q298G | Corynebacterium | Bacillus |
| | sp. Root63 | subtillus | | | | glutamicum | subtillus |
| | | | | | | ATCC 13032 | |
| Sc3A4BAC_87 | Streptomyces | Saccharomyces | P26512 | Aspartokinase | Q298G | Corynebacterium | Saccharomyces |
| | sp. Root63 | cerevisiae | | | | glutamicum | cerevisiae |
| | | | | | | ATCC 13032 | |
| Sc3A4BAC_88 | Streptomyces | Yarrowia | P26512 | Aspartokinase | Q298G | Corynebacterium | Yarrowia |
| | sp. Root63 | lipolytica | | | | glutamicum | lipolytica |
| | | | | | | ATCC 13032 | |
| Sc3A4BAC_89 | Saccharothrix | Saccharomyces | P26512 | Aspartokinase | S301Y | Corynebacterium | Saccharomyces |
| | espanaensis | cerevisiae | | | | glutamicum | cerevisiae |
| | ATCC 51144 | | | | | ATCC 13032 | |
| Sc3A4BAC_90 | Bacillus | Saccharomyces | P26512 | Aspartokinase | S301Y | Corynebacterium | Saccharomyces |
| | cereus | cerevisiae | | | | glutamicum | cerevisiae |
| | | | | | | ATCC 13032 | |
| Sc3A4BAC_91 | Bacillus | Yarrowia | P26512 | Aspartokinase | S301Y | Corynebacterium | Yarrowia |
| | cereus | lipolytica | | | | glutamicum | lipolytica |
| | | | | | | ATCC 13032 | |
| Sc3A4BAC_92 | Streptomyces | Saccharomyces | P26512 | Aspartokinase | S301Y | Corynebacterium | Saccharomyces |
| | sp. Root63 | cerevisiae | | | | glutamicum | cerevisiae |
| | | | | | | ATCC 13032 | |
| Sc3A4BAC_93 | Streptomyces | Yarrowia | P26512 | Aspartokinase | S301Y | Corynebacterium | Yarrowia |
| | sp. Root63 | lipolytica | | | | glutamicum | lipolytica |
| | | | | | | ATCC 13032 | |
| Sc3A4BAC_94 | Saccharothrix | modified | | | | | |
| | espanaensis | Cg codon | | | | | |
| | ATCC 51144 | usage | | | | | |

TABLE 7
Host evaluation - first-round genetic engineering in Corynebacteria glutamicum
Corynebacteria glutamicum expressed enzymes from Host Evaluation round for production of 3-amino-4-hydroxybenzoic acid.

| | | | | | E1 Codon | | |
|---|---|---|---|---|---|---|---|
| | Titer | E1 | Enzyme 1 - | Enzyme 1 - | Optimization | E2 | Enzyme 2 - |
| Strain name | μg/L | Uniprot ID | activity name | source organism | Abbrev. | Uniprot ID | activity name |
| Cg3A4BAC_63 | 36.32 | K0JXI9 | 3-amino-4- | Saccharothrix | Saccharomyces | K0KAK8 | 2-amino-4,5- |
| | | | hydroxybenzoic | espanaensis | cerevisiae | | dihydroxy-6-one- |
| | | | acid synthase | ATCC 51144 | | | heptanoic acid-7- |
| | | | | | | | phosphate |
| | | | | | | | synthase |
| Cg3A4BAC_64 | 0 | K0JXI9 | 3-amino-4- | Saccharothrix | Yarrowia | K0KAK8 | 2-amino-4,5- |
| | | | hydroxybenzoic | espanaensis | lipolytica | | dihydroxy-6-one- |
| | | | acid synthase | ATCC 51144 | | | heptanoic acid-7- |
| | | | | | | | phosphate |
| | | | | | | | synthase |
| Cg3A4BAC_65 | | A0A1T3V8D3 | 3- | Bacillus | Bacillus | NCBI- | 2-amino-4,5- |
| | | | dehydroquinate | anthracis | subtillus | CUB39904.1 | dihydroxy-6-one- |
| | | | synthase | | | | heptanoic acid-7- |
| | | | | | | | phosphate |
| | | | | | | | synthase |
| Cg3A4BAC_66 | 1085.2 | A0A1T3V8D3 | 3- | Bacillus | modified codon | NCBI- | 2-amino-4,5- |
| | | | dehydroquinate | anthracis | usage for Cg | CUB39904.1 | dihydroxy-6-one- |
| | | | synthase | | and Sc | | heptanoic acid-7- |
| | | | | | | | phosphate |
| | | | | | | | synthase |
| Cg3A4BAC_67 | 101.4 | A0A1T3V8D3 | 3- | Bacillus | Saccharomyces | NCBI- | 2-amino-4,5- |
| | | | dehydroquinate | anthracis | cerevisiae | CUB39904.1 | dihydroxy-6-one- |
| | | | synthase | | | | heptanoic acid-7- |
| | | | | | | | phosphate |
| | | | | | | | synthase |
| Cg3A4BAC_68 | 2771.3 | A0A1T3V8D3 | 3- | Bacillus | Yarrowia | NCBI- | 2-amino-4,5- |
| | | | dehydroquinate | anthracis | lipolytica | CUB39904.1 | dihydroxy-6-one- |
| | | | synthase | | | | heptanoic acid-7- |
| | | | | | | | phosphate |
| | | | | | | | synthase |
| Cg3A4BAC_69 | | K0JXI9 | 3-amino-4- | Saccharothrix | Bacillus | A0A0Q8F363 | 2-amino-4,5- |
| | | | hydroxybenzoic | espanaensis | subtillus | | dihydroxy-6-one- |
| | | | acid synthase | ATCC 51144 | | | heptanoic acid-7- |
| | | | | | | | phosphate |
| | | | | | | | synthase |
| Cg3A4BAC_70 | 0 | K0JXI9 | 3-amino-4- | Saccharothrix | modified codon | A0A0Q8F363 | 2-amino-4,5- |
| | | | hydroxybenzoic | espanaensis | usage for Cg | | dihydroxy-6-one- |
| | | | acid synthase | ATCC 51144 | and Sc | | heptanoic acid-7- |
| | | | | | | | phosphate |
| | | | | | | | synthase |
| Cg3A4BAC_71 | 0 | K0JXI9 | 3-amino-4- | Saccharothrix | Saccharomyces | A0A0Q8F363 | 2-amino-4,5- |
| | | | hydroxybenzoic | espanaensis | cerevisiae | | dihydroxy-6-one- |
| | | | acid synthase | ATCC 51144 | | | heptanoic acid-7- |
| | | | | | | | phosphate |
| | | | | | | | synthase |
| Cg3A4BAC_72 | 0 | K0JXI9 | 3-amino-4- | Saccharothrix | Yarrowia | A0A0Q8F363 | 2-amino-4,5- |
| | | | hydroxybenzoic | espanaensis | lipolytica | | dihydroxy-6-one- |
| | | | acid synthase | ATCC 51144 | | | heptanoic acid-7- |
| | | | | | | | phosphate |
| | | | | | | | synthase |
| Cg3A4BAC_73 | 53.4 | K0JXI9 | 3-amino-4- | Saccharothrix | Saccharomyces | K0KAK8 | 2-amino-4,5- |
| | | | hydroxybenzoic | espanaensis | cerevisiae | | dihydroxy-6-one- |
| | | | acid synthase | ATCC 51144 | | | heptanoic acid-7- |
| | | | | | | | phosphate |
| | | | | | | | synthase |
| Cg3A4BAC_74 | 0 | K0JXI9 | 3-amino-4- | Saccharothrix | Yarrowia | K0KAK8 | 2-amino-4,5- |
| | | | hydroxybenzoic | espanaensis | lipolytica | | dihydroxy-6-one- |
| | | | acid synthase | ATCC 51144 | | | heptanoic acid-7- |
| | | | | | | | phosphate |
| | | | | | | | synthase |
| Cg3A4BAC_75 | | A0A1T3V8D3 | 3- | Bacillus | Saccharomyces | NCBI- | 2-amino-4,5- |
| | | | dehydroquinate | anthracis | cerevisiae | CUB39904.1 | dihydroxy-6-one- |
| | | | synthase | | | | heptanoic acid-7- |
| | | | | | | | phosphate |
| | | | | | | | synthase |
| Cg3A4BAC_76 | 2366.1 | A0A1T3V8D3 | 3- | Bacillus | Yarrowia | NCBI- | 2-amino-4,5- |
| | | | dehydroquinate | anthracis | lipolytica | CUB39904.1 | dihydroxy-6-one- |
| | | | synthase | | | | heptanoic acid-7- |
| | | | | | | | phosphate |
| | | | | | | | synthase |
| Cg3A4BAC_77 | 0 | K0JXI9 | 3-amino-4- | Saccharothrix | Saccharomyces | A0A0Q8F363 | 2-amino-4,5- |
| | | | hydroxybenzoic | espanaensis | cerevisiae | | dihydroxy-6-one- |
| | | | acid synthase | ATCC 51144 | | | heptanoic acid-7- |
| | | | | | | | phosphate |
| | | | | | | | synthase |
| Cg3A4BAC_78 | 0 | K0JXI9 | 3-amino-4- | Saccharothrix | Yarrowia | A0A0Q8F363 | 2-amino-4,5- |
| | | | hydroxybenzoic | espanaensis | lipolytica | | dihydroxy-6-one- |
| | | | acid synthase | ATCC 51144 | | | heptanoic acid-7- |
| | | | | | | | phosphate |
| | | | | | | | synthase |

| | | E2 Codon | | | E3 | | E3 Codon |
|---|---|---|---|---|---|---|---|
| | Enzyme 2 - | Optimization | E3 | Enzyme 3 - | Modifi- | Enzyme 3 - | Optimization |
| Strain name | source organism | Abbrev. | Uniprot ID | activity name | cations | source organism | Abbrev. |
| Cg3A4BAC_63 | Saccharothrix | Saccharomyces | P26512 | Aspartokinase | Q298G | Corynebacterium | Saccharomyces |
| | espanaensis | cerevisiae | | | | glutamicum | cerevisiae |
| | ATCC 51144 | | | | | ATCC 13032 | |
| Cg3A4BAC_64 | Saccharothrix | Yarrowia | P26512 | Aspartokinase | Q298G | Corynebacterium | Yarrowia |
| | espanaensis | lipolytica | | | | glutamicum | lipolytica |
| | ATCC 51144 | | | | | ATCC 13032 | |
| Cg3A4BAC_65 | Bacillus | Bacillus | P26512 | Aspartokinase | Q298G | Corynebacterium | Bacillus |
| | cereus | subtillus | | | | glutamicum | subtillus |
| | | | | | | ATCC 13032 | |
| Cg3A4BAC_66 | Bacillus | modified codon | | | | | |
| | cereus | usage for Cg | | | | | |
| | | and Sc | | | | | |
| Cg3A4BAC_67 | Bacillus | Saccharomyces | P26512 | Aspartokinase | Q298G | Corynebacterium | Saccharomyces |
| | cereus | cerevisiae | | | | glutamicum | cerevisiae |
| | | | | | | ATCC 13032 | |
| Cg3A4BAC_68 | Bacillus | Yarrowia | P26512 | Aspartokinase | Q298G | Corynebacterium | Yarrowia |
| | cereus | lipolytica | | | | glutamicum | lipolytica |
| | | | | | | ATCC 13032 | |
| Cg3A4BAC_69 | Streptomyces | Bacillus | P26512 | Aspartokinase | Q298G | Corynebacterium | Bacillus |
| | sp. Root63 | subtillus | | | | glutamicum | subtillus |
| | | | | | | ATCC 13032 | |
| Cg3A4BAC_70 | Streptomyces | modified codon | | | | | |
| | sp. Root63 | usage for Cg | | | | | |
| | | and Sc | | | | | |
| Cg3A4BAC_71 | Streptomyces | Saccharomyces | P26512 | Aspartokinase | Q298G | Corynebacterium | Saccharomyces |
| | sp. Root63 | cerevisiae | | | | glutamicum | cerevisiae |
| | | | | | | ATCC 13032 | |
| Cg3A4BAC_72 | Streptomyces | Yarrowia | P26512 | Aspartokinase | Q298G | Corynebacterium | Yarrowia |
| | sp. Root63 | lipolytica | | | | glutamicum | lipolytica |
| | | | | | | ATCC 13032 | |
| Cg3A4BAC_73 | Saccharothrix | Saccharomyces | P26512 | Aspartokinase | S301Y | Corynebacterium | Saccharomyces |
| | espanaensis | cerevisiae | | | | glutamicum | cerevisiae |
| | ATCC 51144 | | | | | ATCC 13032 | |
| Cg3A4BAC_74 | Saccharothrix | Yarrowia | P26512 | Aspartokinase | S301Y | Corynebacterium | Yarrowia |
| | espanaensis | lipolytica | | | | glutamicum | lipolytica |
| | ATCC 51144 | | | | | ATCC 13032 | |
| Cg3A4BAC_75 | Bacillus | Saccharomyces | P26512 | Aspartokinase | S301Y | Corynebacterium | Saccharomyces |
| | cereus | cerevisiae | | | | glutamicum | cerevisiae |
| | | | | | | ATCC 13032 | |
| Cg3A4BAC_76 | Bacillus | Yarrowia | P26512 | Aspartokinase | S301Y | Corynebacterium | Yarrowia |
| | cereus | lipolytica | | | | glutamicum | lipolytica |
| | | | | | | ATCC 13032 | |
| Cg3A4BAC_77 | Streptomyces | Saccharomyces | P26512 | Aspartokinase | S301Y | Corynebacterium | Saccharomyces |
| | sp. Root63 | cerevisiae | | | | glutamicum | cerevisiae |
| | | | | | | ATCC 13032 | |
| Cg3A4BAC_78 | Streptomyces | Yarrowia | P26512 | Aspartokinase | S301Y | Corynebacterium | Yarrowia |
| | sp. Root63 | lipolytica | | | | glutamicum | lipolytica |
| | | | | | | ATCC 13032 | |

TABLE 8
Improvement-round genetic engineering in Saccharomyces cerevisiae
Saccharomyces cerevisiae strains expressed enzymes as indicated in the table below. In addition to the
genetic changes in the table below, the strains also contained enzymes from first round:
2-amino-4,5-dihydroxy-6-oxo-7-(phosphooxy)heptanoate synthase (UniProt ID A0A0Q8F363) from Streptomyces sp.
Root63, and 3-amino-4-benzoic acid synthase (UniProt ID K0JXI9) from Saccharothrix espanaensis ATCC 51144.

| | | | | E1 | | E1 Codon | | |
|---|---|---|---|---|---|---|---|---|
| | Titer | E1 | Enzyme 1 - | Modifi- | Enzyme 1 - | Optimization | E2 | Enzyme 2 - |
| Strain name | μg/L | Uniprot ID | activity name | cations | source organism | Abbrev. | Uniprot ID | activity name |
| Sc3A4BAC_09 | 0 | A0A0Q8F363 | 2-amino-4,5- | | Streptomyces | modified | K0JXI9 | 3-amino-4- |
| | | | dihydroxy- | | sp. Root63 | Cg codon | | benzoic acid |
| | | | 6-oxo-7- | | | usage | | synthase |
| | | | (phosphor- | | | | | |
| | | | oxy) | | | | | |
| | | | heptanoate | | | | | |
| | | | synthase | | | | | |
| Sc3A4BAC_32 | 0 | A4QEF2 | Glucose-6- | A243T | Corynebacterium | modified codon | | |
| | | | phosphate 1- | | glutamicum | usage for Cg | | |
| | | | dehydrogenase | | (strain R) | and Sc | | |
| Sc3A4BAC_33 | 0 | Q34425 | Cytochrome b | | Makalata | modified codon | | |
| | | | (Fragment) | | didelphoides | usage for Cg | | |
| | | | | | | and Sc | | |
| Sc3A4BAC_34 | 0 | P12880 | Phospho- | D299N | Corynebacterium | modified codon | | |
| | | | enolpyruvate | | glutamicum | usage for Cg | | |
| | | | carboxylase | | ATCC 13032 | and Sc | | |
| Sc3A4BAC_35 | 0 | P12880 | Phospho- | N917G | Corynebacterium | modified codon | | |
| | | | enolpyruvate | | glutamicum | usage for Cg | | |
| | | | carboxylase | | ATCC 13032 | and Sc | | |
| Sc3A4BAC_36 | 14.47 | P26512 | Aspartokinase | Q298G | Corynebacterium | modified codon | P12880 | Phospho- |
| | | | | | glutamicum | usage for Cg | | enolpyruvate |
| | | | | | ATCC 13032 | and Sc | | carboxylase |
| Sc3A4BAC_37 | 0 | P26512 | Aspartokinase | S301Y | Corynebacterium | modified codon | P12880 | Phospho- |
| | | | | | glutamicum | usage for Cg | | enolpyruvate |
| | | | | | ATCC 13032 | and Sc | | carboxylase |
| Sc3A4BAC_38 | 13.44 | P26512 | Aspartokinase | Q298G | Corynebacterium | modified codon | P12880 | Phospho- |
| | | | | | glutamicum | usage for Cg | | enolpyruvate |
| | | | | | ATCC 13032 | and Sc | | carboxylase |
| Sc3A4BAC_39 | 0 | P26512 | Aspartokinase | Q298G | Corynebacterium | modified codon | H7C7K2 | Pyruvate |
| | | | | | glutamicum | usage for Cg | | carboxylase |
| | | | | | ATCC 13032 | and Sc | | |
| Sc3A4BAC_40 | 0 | P26512 | Aspartokinase | S301Y | Corynebacterium | modified codon | H7C7K2 | Pyruvate |
| | | | | | glutamicum | usage for Cg | | carboxylase |
| | | | | | ATCC 13032 | and Sc | | |
| Sc3A4BAC_41 | 0 | P26512 | Aspartokinase | Q298G | Corynebacterium | modified codon | H7C7K2 | Pyruvate |
| | | | | | glutamicum | usage for Cg | | carboxylase |
| | | | | | ATCC 13032 | and Sc | | |
| Sc3A4BAC_42 | 0 | P26512 | Aspartokinase | S301Y | Corynebacterium | modified codon | H7C7K2 | Pyruvate |
| | | | | | glutamicum | usage for Cg | | carboxylase |
| | | | | | ATCC 13032 | and Sc | | |
| Sc3A4BAC_43 | 11.77 | P26512 | Aspartokinase | Q298G | Corynebacterium | modified codon | P0C1D8 | Aspartate- |
| | | | | | glutamicum | usage for Cg | | semialdehyde |
| | | | | | ATCC 13032 | and Sc | | dehydrogenase |
| Sc3A4BAC_44 | 0 | P26512 | Aspartokinase | S301Y | Corynebacterium | modified codon | P07262 | NADP-specific |
| | | | | | glutamicum | usage for Cg | | glutamate |
| | | | | | ATCC 13032 | and Sc | | dehydrogenase |
| Sc3A4BAC_45 | 0 | P0C1D8 | Aspartate- | | Corynebacterium | modified codon | Q12680 | Glutamate |
| | | | semialdehyde | | glutamicum | usage for Cg | | synthase |
| | | | dehydrogenase | | ATCC 13032 | and Sc | | |
| Sc3A4BAC_46 | 0 | P13663 | Aspartate- | | Saccharomyces | modified codon | P32288 | Glutamine |
| | | | semialdehyde | | cerevisiae | usage for Cg | | synthetase |
| | | | dehydrogenase | | S288c | and Sc | | |
| Sc3A4BAC_47 | 0 | P0C1D8 | Aspartate- | | Corynebacterium | modified codon | Q12680 | Glutamate |
| | | | semialdehyde | | glutamicum | usage for Cg | | synthase |
| | | | dehydrogenase | | ATCC 13032 | and Sc | | |
| Sc3A4BAC_49 | 0 | P13663 | Aspartate- | | Saccharomyces | modified codon | Q12680 | Glutamate |
| | | | semialdehyde | | cerevisiae | usage for Cg | | synthase |
| | | | dehydrogenase | | S288c | and Sc | | |
| Sc3A4BAC_50 | 0 | P13663 | Aspartate- | | Saccharomyces | modified codon | P09832 | Glutamate |
| | | | semialdehyde | | cerevisiae | usage for Cg | | synthase |
| | | | dehydrogenase | | S288c | and Sc | | |
| Sc3A4BAC_51 | 0 | P13663 | Aspartate- | | Saccharomyces | modified codon | P32288 | Glutamine |
| | | | semialdehyde | | cerevisiae | usage for Cg | | synthetase |
| | | | dehydrogenase | | S288c | and Sc | | |
| Sc3A4BAC_52 | 0 | A0JC77 | 2-amino-4,5- | | Streptomyces | modified | A0JC76 | 3-amino-4- |
| | | | dihydroxy- | | griseus | Corynebacterium | | hydroxybenzoic |
| | | | 6-oxo-7- | | | glutamicum | | acid synthase |
| | | | (phosphor- | | | codon usage | | |
| | | | oxy) | | | | | |
| | | | heptanoate | | | | | |
| | | | synthase | | | | | |
| Sc3A4BAC_53 | 10.37 | A0JC77 | 2-amino-4,5- | | Streptomyces | modified | A0A101UF72 | 3- |
| | | | dihydroxy- | | griseus | Corynebacterium | | dehydroquinate |
| | | | 6-oxo-7- | | | glutamicum | | synthase |
| | | | (phosphor- | | | codon usage | | |
| | | | oxy) | | | | | |
| | | | heptanoate | | | | | |
| | | | synthase | | | | | |
| Sc3A4BAC_54 | 2.64 | A0JC77 | 2-amino-4,5- | | Streptomyces | modified | NCBI- | 3-amino-4- |
| | | | dihydroxy- | | griseus | Corynebacterium | WP_055705152 | hydroxybenzoic |
| | | | 6-oxo-7- | | | glutamicum | | acid synthase |
| | | | (phosphor- | | | codon usage | | |
| | | | oxy) | | | | | |
| | | | heptanoate | | | | | |
| | | | synthase | | | | | |
| Sc3A4BAC_55 | 8.6 | A0JC77 | 2-amino-4,5- | | Streptomyces | modified | F3NEM9 | 3- |
| | | | dihydroxy- | | griseus | Corynebacterium | | dehydroquinate |
| | | | 6-oxo-7- | | | glutamicum | | synthase/3,4- |
| | | | (phosphor- | | | codon usage | | AHBA synthase |
| | | | oxy) | | | | | |
| | | | heptanoate | | | | | |
| | | | synthase | | | | | |
| Sc3A4BAC_56 | 7.78 | A0JC77 | 2-amino-4,5- | | Streptomyces | modified | A0A1Q4XIX7 | 3- |
| | | | dihydroxy- | | griseus | Corynebacterium | | dehydroquinate |
| | | | 6-oxo-7- | | | glutamicum | | synthase |
| | | | (phosphor- | | | codon usage | | |
| | | | oxy) | | | | | |
| | | | heptanoate | | | | | |
| | | | synthase | | | | | |
| Sc3A4BAC_57 | 8.36 | A0JC77 | 2-amino-4,5- | | Streptomyces | modified | NCBI- | 3-amino-4- |
| | | | dihydroxy- | | griseus | Corynebacterium | WP_010696313 | hydroxybenzoic |
| | | | 6-oxo-7- | | | glutamicum | | acid synthase |
| | | | (phosphor- | | | codon usage | | |
| | | | oxy) | | | | | |
| | | | heptanoate | | | | | |
| | | | synthase | | | | | |
| Sc3A4BAC_58 | 0 | A0JC77 | 2-amino-4,5- | | Streptomyces | modified | NCBI- | 3-amino-4- |
| | | | dihydroxy- | | griseus | Corynebacterium | WP_033531253 | hydroxybenzoic |
| | | | 6-oxo-7- | | | glutamicum | | acid synthase |
| | | | (phosphor- | | | codon usage | | |
| | | | oxy) | | | | | |
| | | | heptanoate | | | | | |
| | | | synthase | | | | | |
| Sc3A4BAC_60 | 8.09 | A0JC77 | 2-amino-4,5- | | Streptomyces | modified | A0A088DA72 | Putative 3,4- |
| | | | dihydroxy- | | griseus | Corynebacterium | | AHBA synthase |
| | | | 6-oxo-7- | | | glutamicum | | |
| | | | (phosphor- | | | codon usage | | |
| | | | oxy)heptanoate | | | | | |
| | | | synthase | | | | | |
| Sc3A4BAC_61 | 0 | A0JC77 | 2-amino-4,5- | | Streptomyces | modified | NCBI- | 3-amino-4- |
| | | | dihydroxy- | | griseus | Corynebacterium | WP_078864288 | hydroxybenzoic |
| | | | 6-oxo-7- | | | glutamicum | | acid synthase |
| | | | (phosphor- | | | codon usage | | |
| | | | oxy) | | | | | |
| | | | heptanoate | | | | | |
| | | | synthase | | | | | |
| Sc3A4BAC_62 | 752.5 | NCBI- | 2-amino-4,5- | | Bacteroidetes | modified codon | A0JC76 | 3-amino-4- |
| | | OFX87022 | dihydroxy- | | bacterium | usage for Cg | | hydroxybenzoic |
| | | | 6-one- | | GWE2_32_14 | and Sc | | acid synthase |
| | | | heptanoic | | | | | |
| | | | acid-7- | | | | | |
| | | | phosphate | | | | | |
| | | | synthase | | | | | |
| Sc3A4BAC_63 | 2.68 | NCBI- | Deoxyribose- | | Clostridium | modified codon | A0JC76 | 3-amino-4- |
| | | WP_024831757 | phosphate | | cellulolyticum | usage for Cg | | hydroxybenzoic |
| | | | aldolase/ | | | and Sc | | acid synthase |
| | | | phospho-2- | | | | | |
| | | | dehydro-3- | | | | | |
| | | | deoxyheptonate | | | | | |
| | | | aldolase | | | | | |
| Sc3A4BAC_65 | 0 | A0A1W2FM87 | 2-amino-4,5- | | Lentzea | modified codon | A0JC76 | 3-amino-4- |
| | | | dihydroxy- | | albidocapillata | usage for Cg | | hydroxybenzoic |
| | | | 6-oxo-7- | | | and Sc | | acid synthase |
| | | | (Phosphono- | | | | | |
| | | | oxy) | | | | | |
| | | | heptanoate | | | | | |
| | | | synthase | | | | | |
| Sc3A4BAC_66 | 0 | NCBI- | 2-amino-4,5- | | Lentzea | modified codon | A0JC76 | 3-amino-4- |
| | | SDG98226 | dihydroxy- | | albidocapillata | usage for Cg | | hydroxybenzoic |
| | | | 6-one- | | | and Sc | | acid synthase |
| | | | heptanoic | | | | | |
| | | | acid-7- | | | | | |
| | | | phosphate | | | | | |
| | | | synthase | | | | | |
| Sc3A4BAC_67 | 20.24 | A0A209CVJ1 | Aspartate | | Streptomyces | modified codon | A0JC76 | 3-amino-4- |
| | | | kinase | | sp. CS057 | usage for Cg | | hydroxybenzoic |
| | | | | | | and Sc | | acid synthase |
| Sc3A4BAC_68 | 3261 | A0A132MRF8 | 2-amino-4 | | Streptomyces | modified codon | A0JC76 | 3-amino-4- |
| | | | (2-amino-4,5- | | thermoauto- | usage for Cg | | hydroxybenzoic |
| | | | dihydroxy- | | trophicus | and Sc | | acid synthase |
| | | | 6-one- | | | | | |
| | | | heptanoic | | | | | |
| | | | acid-7- | | | | | |
| | | | phosphate | | | | | |
| | | | synthase) | | | | | |
| Sc3A4BAC_69 | 0 | NCBI- | 2-amino-4,5- | | Streptomyces | modified codon | A0JC76 | 3-amino-4- |
| | | SDZ37301 | dihydroxy- | | | usage for Cg | | hydroxybenzoic |
| | | | 6-one- | | | and Sc | | acid synthase |
| | | | heptanoic | | | | | |
| | | | acid-7- | | | | | |
| | | | phosphate | | | | | |
| | | | synthase | | | | | |
| Sc3A4BAC_70 | 2.78 | P23538 | Phospho- | | Escherichia | modified codon | | |
| | | | enolpyruvate | | coli | usage for Cg | | |
| | | | synthase | | (strain K12) | and Sc | | |
| Sc3A4BAC_72 | 0 | P00509 | aspartate | | Escherichia | modified codon | P26512 | Aspartokinase |
| | | | amino- | | coli | usage for Cg | | |
| | | | transferase | | (strain K12) | and Sc | | |
| | | | activity | | | | | |
| Sc3A4BAC_73 | 0 | P00509 | aspartate | | Escherichia | modified codon | P26512 | Aspartokinase |
| | | | amino- | | coli | usage for Cg | | |
| | | | transferase | | (strain K12) | and Sc | | |
| | | | activity | | | | | |
| Sc3A4BAC_74 | 0 | P00509 | aspartate | | Escherichia | modified codon | P26512 | Aspartokinase |
| | | | amino- | | coli | usage for Cg | | |
| | | | transferase | | (strain K12) | and Sc | | |
| | | | activity | | | | | |
| Sc3A4BAC_75 | 0 | P00509 | aspartate | | Escherichia | modified codon | P26512 | Aspartokinase |
| | | | amino- | | coli | usage for Cg | | |
| | | | transferase | | (strain K12) | and Sc | | |
| | | | activity | | | | | |
| Sc3A4BAC_76 | 0 | P00509 | aspartate | | Escherichia | modified codon | P26512 | Aspartokinase |
| | | | amino- | | coli | usage for Cg | | |
| | | | transferase | | (strain K12) | and Sc | | |
| | | | activity | | | | | |
| Sc3A4BAC_77 | 0 | P00509 | aspartate | | Escherichia | modified codon | P0A9Q9 | Aspartate- |
| | | | amino- | | coli | usage for Cg | | semialdehyde |
| | | | transferase | | (strain K12) | and Sc | | dehydrogenase |
| | | | activity | | | | | |
| Sc3A4BAC_78 | | P00509 | aspartate | | Escherichia | modified codon | P0A9Q9 | Aspartate- |
| | | | amino- | | coli | usage for Cg | | semialdehyde |
| | | | transferase | | (strain K12) | and Sc | | dehydrogenase |
| | | | activity | | | | | |

| | E2 | | E2 Codon | | | | E3 Codon |
|---|---|---|---|---|---|---|---|
| | Modifi- | Enzyme 2 - | Optimization | E3 | Enzyme 3 - | Enzyme 3 - | Optimization |
| Strain name | cations | source organism | Abbrev. | Uniprot ID | activity name | source organism | Abbrev. |
| Sc3A4BAC_09 | | Saccharothrix | modified | | | | |
| | | espanaensis | Corynebacterium | | | | |
| | | ATCC 51144 | glutamicum | | | | |
| | | | codon usage | | | | |
| Sc3A4BAC_32 | | | | | | | |
| Sc3A4BAC_33 | | | | | | | |
| Sc3A4BAC_34 | | | | | | | |
| Sc3A4BAC_35 | | Corynebacterium | | | | | |
| | | glutamicum | | | | | |
| | | ATCC 13032 | | | | | |
| Sc3A4BAC_36 | N917G | Corynebacterium | modified codon | | | | |
| | | glutamicum | usage for Cg | | | | |
| | | ATCC 13032 | and Sc | | | | |
| Sc3A4BAC_37 | N917G | Corynebacterium | modified codon | | | | |
| | | glutamicum | usage for Cg | | | | |
| | | ATCC 13032 | and Sc | | | | |
| Sc3A4BAC_38 | N917G | Corynebacterium | modified codon | P00509 | aspartate | Escherichia | modified codon |
| | | glutamicum | usage for Cg | | amino- | coli | usage for Cg |
| | | ATCC 13032 | and Sc | | transferase | K12 | and Sc |
| | | | | | activity | | |
| Sc3A4BAC_39 | P458S | Corynebacterium | modified codon | | | | |
| | | glutamicum | usage for Cg | | | | |
| | | ATCC 13032 | and Sc | | | | |
| Sc3A4BAC_40 | P458S | Corynebacterium | modified codon | | | | |
| | | glutamicum | usage for Cg | | | | |
| | | ATCC 13032 | and Sc | | | | |
| Sc3A4BAC_41 | P458S | Corynebacterium | modified codon | P00509 | aspartate | Escherichia | modified codon |
| | | glutamicum | usage for Cg | | amino- | coli | usage for Cg |
| | | ATCC 13032 | and Sc | | transferase | K12 | and Sc |
| | | | | | activity | | |
| Sc3A4BAC_42 | P458S | Corynebacterium | modified codon | P00509 | aspartate | Escherichia | modified codon |
| | | glutamicum | usage for Cg | | amino- | coli | usage for Cg |
| | | ATCC 13032 | and Sc | | transferase | K12 | and Sc |
| | | | | | activity | | |
| Sc3A4BAC_43 | | Corynebacterium | modified codon | P00509 | aspartate | Escherichia | modified codon |
| | | glutamicum | usage for Cg | | amino- | coli | usage for Cg |
| | | ATCC 13032 | and Sc | | transferase | K12 | and Sc |
| | | | | | activity | | |
| Sc3A4BAC_44 | | Saccharomyces | modified codon | P00509 | aspartate | Escherichia | modified codon |
| | | cerevisiae | usage for Cg | | amino- | coli | usage for Cg |
| | | S288c | and Sc | | transferase | K12 | and Sc |
| | | | | | activity | | |
| Sc3A4BAC_45 | | Saccharomyces | modified codon | P00509 | aspartate | Escherichia | modified codon |
| | | cerevisiae | usage for Cg | | amino- | coli | usage for Cg |
| | | S288c | and Sc | | transferase | K12 | and Sc |
| | | | | | activity | | |
| Sc3A4BAC_46 | | Saccharomyces | modified codon | P00509 | aspartate | Escherichia | modified codon |
| | | cerevisiae | usage for Cg | | amino- | coli | usage for Cg |
| | | S288c | and Sc | | transferase | K12 | and Sc |
| | | | | | activity | | |
| Sc3A4BAC_47 | | Saccharomyces | modified codon | | | | |
| | | cerevisiae | usage for Cg | | | | |
| | | S288c | and Sc | | | | |
| Sc3A4BAC_49 | | Saccharomyces | modified codon | | | | |
| | | cerevisiae | usage for Cg | | | | |
| | | S288c | and Sc | | | | |
| Sc3A4BAC_50 | | Escherichia | modified codon | P09831 | Glutamate | Escherichia | modified codon |
| | | coli | usage for Cg | | synthase | coli | usage for Cg |
| | | (strain K12) | and Sc | | [NADPH] | K12 | and Sc |
| | | | | | large chain | | |
| | | | | | (EC 1.4.1.13) | | |
| | | | | | (Glutamate | | |
| | | | | | synthase | | |
| | | | | | subunit | | |
| | | | | | alpha) | | |
| | | | | | (GLTS | | |
| | | | | | alpha chain) | | |
| | | | | | (NADPH- | | |
| | | | | | GOGAT) | | |
| Sc3A4BAC_51 | | Saccharomyces | modified codon | | | | |
| | | cerevisiae | usage for Cg | | | | |
| | | S288c | and Sc | | | | |
| Sc3A4BAC_52 | | Streptomyces | modified codon | | | | |
| | | griseus | usage for Cg | | | | |
| | | | and Sc | | | | |
| Sc3A4BAC_53 | | Streptomyces | modified codon | | | | |
| | | sp. DSM 15324 | usage for Cg | | | | |
| | | | and Sc | | | | |
| Sc3A4BAC_54 | | Streptomyces | modified codon | | | | |
| | | | usage for Cg | | | | |
| | | | and Sc | | | | |
| Sc3A4BAC_55 | | Streptomyces | modified codon | | | | |
| | | griseoaurantiacus | usage for Cg | | | | |
| | | M045 | and Sc | | | | |
| Sc3A4BAC_56 | | Streptomyces | modified codon | | | | |
| | | sp. CB03911 | usage for Cg | | | | |
| | | | and Sc | | | | |
| Sc3A4BAC_57 | | Streptomyces | modified codon | | | | |
| | | atratus | usage for Cg | | | | |
| | | | and Sc | | | | |
| Sc3A4BAC_58 | | Streptomyces | modified codon | | | | |
| | | galbus | usage for Cg | | | | |
| | | | and Sc | | | | |
| Sc3A4BAC_60 | | Streptomyces | modified codon | | | | |
| | | aureus | usage for Cg | | | | |
| | | | and Sc | | | | |
| Sc3A4BAC_61 | | Streptomyces | modified codon | | | | |
| | | sp | usage for Cg | | | | |
| | | | and Sc | | | | |
| Sc3A4BAC_62 | | Streptomyces | modified codon | | | | |
| | | griseus | usage for Cg | | | | |
| | | | and Sc | | | | |
| Sc3A4BAC_63 | | Streptomyces | modified codon | | | | |
| | | griseus | usage for Cg | | | | |
| | | | and Sc | | | | |
| Sc3A4BAC_65 | | Streptomyces | modified codon | | | | |
| | | griseus | usage for Cg | | | | |
| | | | and Sc | | | | |
| Sc3A4BAC_66 | | Streptomyces | modified codon | | | | |
| | | griseus | usage for Cg | | | | |
| | | | and Sc | | | | |
| Sc3A4BAC_67 | | Streptomyces | modified codon | | | | |
| | | griseus | usage for Cg | | | | |
| | | | and Sc | | | | |
| Sc3A4BAC_68 | | Streptomyces | modified codon | | | | |
| | | griseus | usage for Cg | | | | |
| | | | and Sc | | | | |
| Sc3A4BAC_69 | | Streptomyces | modified codon | | | | |
| | | griseus | usage for Cg | | | | |
| | | | and Sc | | | | |
| Sc3A4BAC_70 | | | | | | | |
| Sc3A4BAC_72 | Q298G | Corynebacterium | modified codon | P0A9Q9 | Aspartate- | Escherichia | modified codon |
| | | glutamicum | usage for Cg | | semialdehyde | coli | usage for Cg |
| | | ATCC 13032 | and Sc | | dehydrogenase | K12 | and Sc |
| | | | | | (ASA | | |
| | | | | | dehydrogenase) | | |
| | | | | | (ASADH) | | |
| | | | | | (EC 1.2.1.11) | | |
| | | | | | (Aspartate- | | |
| | | | | | beta- | | |
| | | | | | semialdehyde | | |
| | | | | | dehydrogenase) | | |
| Sc3A4BAC_73 | S301Y | Corynebacterium | modified codon | P0A9Q9 | Aspartate- | Escherichia | modified codon |
| | | glutamicum | usage for Cg | | semialdehyde | coli | usage for Cg |
| | | ATCC 13032 | and Sc | | dehydrogenase | K12 | and Sc |
| | | | | | (ASA | | |
| | | | | | dehydrogenase) | | |
| | | | | | (ASADH) | | |
| | | | | | (EC 1.2.1.11) | | |
| | | | | | (Aspartate- | | |
| | | | | | beta- | | |
| | | | | | semialdehyde | | |
| | | | | | dehydrogenase) | | |
| Sc3A4BAC_74 | S301Y | Corynebacterium | modified codon | P0C1D8 | Aspartate- | Corynebacterium | modified codon |
| | | glutamicum | usage for Cg | | semialdehyde | glutamicum | usage for Cg |
| | | ATCC 13032 | and Sc | | dehydrogenase | ATCC 13032 | and Sc |
| | | | | | (ASA | | |
| | | | | | dehydrogenase) | | |
| | | | | | (ASADH) | | |
| Sc3A4BAC_75 | Q298G | Corynebacterium | modified codon | P23538 | Phospho- | Escherichia | modified codon |
| | | glutamicum | usage for Cg | | enolpyruvate | coli | usage for Cg |
| | | ATCC 13032 | and Sc | | synthase | K12 | and Sc |
| | | | | | (PEP synthase) | | |
| | | | | | (EC 2.7.9.2) | | |
| | | | | | (Pyruvate, | | |
| | | | | | water dikinase) | | |
| Sc3A4BAC_76 | S301Y | Corynebacterium | modified codon | P22259 | Phospho- | Escherichia | modified codon |
| | | glutamicum | usage for Cg | | enolpyruvate | coli | usage for Cg |
| | | ATCC 13032 | and Sc | | carboxy- | K12 | and Sc |
| | | | | | kinase | | |
| | | | | | (ATP) | | |
| | | | | | (PCK) | | |
| | | | | | (PEP | | |
| | | | | | carboxy- | | |
| | | | | | kinase) | | |
| | | | | | (PEPCK) | | |
| | | | | | (EC 4.1.1.49) | | |
| Sc3A4BAC_77 | 0 | Escherichia | modified codon | P23538 | Phospho- | Escherichia | modified codon |
| | | coli | usage for Cg | | enolpyruvate | coli | usage for Cg |
| | | (strain K12) | and Sc | | synthase | K12 | and Sc |
| | | | | | (PEP synthase) | | |
| | | | | | (EC 2.7.9.2) | | |
| | | | | | (Pyruvate, | | |
| | | | | | water dikinase) | | |
| Sc3A4BAC_78 | 0 | Escherichia | modified codon | P22259 | Phospho- | Escherichia | modified codon |
| | | coli | usage for Cg | | enolpyruvate | coli | usage for Cg |
| | | (strain K12) | and Sc | | carboxy- | K12 | and Sc |
| | | | | | kinase | | |
| | | | | | (ATP) | | |
| | | | | | (PCK) | | |
| | | | | | (PEP | | |
| | | | | | carboxy- | | |
| | | | | | kinase) | | |
| | | | | | (PEPCK) | | |
| | | | | | (EC 4.1.1.49) | | |

### REFERENCES

1. Bertasso, M., et al., Bagremycin A and B, novel antibiotics from streptomyces sp. Tu 4128. J Antibiot (Tokyo), 2001. 54(9): p. 730-6.
2. Hu, Y. and H. G. Floss, Further studies on the biosynthesis of the manumycin-type antibiotic, asukamycin, and the chemical synthesis of protoasukamycin. J Am Chem Soc, 2004. 126(12): p. 3837-44.
3. Rui, Z., et al., Biochemical and genetic insights into asukamycin biosynthesis. J Biol Chem, 2010. 285(32): p. 24915-24.
4. Suzuki, H., et al., Novel benzene ring biosynthesis from C(3) and C(4) primary metabolites by two enzymes. J Biol Chem, 2006. 281(48): p. 36944-51.
5. Suzuki, H., Y. Ohnishi, and S. Horinouchi, GriC and GriD constitute a carboxylic acid reductase involved in grixazone biosynthesis in Streptomyces griseus. J Antibiot (Tokyo), 2007. 60(6): p. 380-7.
6. Kawaguchi, H., et al., 3-Amino-4-hydroxybenzoic acid production from sweet sorghum juice by recombinant Corynebacterium glutamicum. Bioresour Technol, 2015. 198: p. 410-7.
7. Zelle, R. M., et al., Malic acid production by Saccharomyces cerevisiae: engineering of pyruvate carboxylation, oxaloacetate reduction, and malate export. Appl Environ Microbiol, 2008. 74(9): p. 2766-77.
8. Klein, M., et al., Glycerol metabolism and transport in yeast and fungi: established knowledge and ambiguities. Environ Microbiol, 2017. 19(3): p. 878-893.
9. Zhang, A., et al., Effect of FPS1 deletion on the fermentation properties of Saccharomyces cerevisiae. Lett Appl Microbiol, 2007. 44(2): p. 212-7.
10. Oliveira, R., et al., Fps1p channel is the mediator of the major part of glycerol passive diffusion in Saccharomyces cerevisiae: artefacts and re-definitions. Biochim Biophys Acta, 2003. 1613(1-2): p. 57-71.
11. Wu et al. Efficient mining of natural NADH-utilizing dehydrogenases enables systematic cofactor engineering of lysine synthesis pathway of Corynebacterium glutamicum. Metabolic Engineering (2019) 52:77-86.
12. Cahn et al. A General Tool for Engineering the NAD/NADP Cofactor Preference of Oxidoreductases. ACS Synth. Biol. (2017) 6(2) 326-333.
13. Sugio, T et al. NADH-dependent Sulfite Reductase Activity in the Periplasmic Space of Thiobacillusferrooxidans. Bioscience, Biotechnology, and Biochemistry (1993) 57(8): 1357-1359.
14. Hallenbeck P., et al. Characterization of anaerobic sulfite reduction by Salmonella typhimurium and purification of the anaerobically induced sulfite reductase. J. Bacteriology (1989) 171(6): 3008-3015.
15. Stincone et al., The return of metabolism: biochemistry and physiology of the pentose phosphate pathway. Biol Rev Camb Philos Soc. (2015) 90(3): 927-963.

INFORMAL SEQUENCE LISTING
INFORMAL SEQUENCE LISTING
<1; Protein/1;2-amino-4,5-dihydroxy-6-oxo-7-
(phosphooxy)heptanoate synthase (A0A0Q8F363)
from Streptomyces sp. Root63>
MTLNGSFGARLRGRKLYRNSPDRLLVVPLDHSVTD
GPIATAAGLNHLVGRLSDNGVDAIVLHKGTLRRLD
PEPFTRTSLIVHLSVSTMHAPDPDAKYLVSDVENA
LRLGADAVSVHVNVGSAGEAAQVADMAAVADACDR
WNIPLLAMMYPRGPEISDPRDLVLVKHVATLAADL
GADLVKVPCPRKVTDLADVVSACPVPVLVAGGQVA
GTTEELLDAVGGILDTGVGGLAMGRNIFQADDPGK
RARQVADLVHAPPLRYGPAGTPGPAPHRLP
<2; Protein/1;3-amino-4-benzoic acid
synthase (K0JXI9) from
Saccharothrix espanaensis
ATCC 51144>
MPPGHRIRPDRRASPHWNAVGGWFGVKFAWIDIRA
VDARHREAVVDAAVHAGLGGVLDHRLETLATLPPT
VTKVLLPGPGEVPPAEAAGACDWLTRVATFTELDK
LKLVAGEVDAHAGAFVEVVDDLTLRVACAAVQALE
HTVVRFRDPTKIPLEIVIAAADRAPGLLVCEADGI
EEARIVLDVLEKGSDGLLVAPRDANDVFDVDKLLR
TATPDLALTTLTVRSVEHNGLGDRICVDTCTHFRE
DEGILVGSFAHGFVLCVSETHPLPYMPTRPFRVNA
GALHSYVFGADNRTNYLSELKAGSTVLGVTADGRT
RRIVVGRVKLESRPLLTVHATAPDGTEVALTLQDD
WHVRVLGPGAAVLNSTELEPGDQLLGYLATDKRHV
GWPVGEFCIEK
<3; Protein/1;2-amino-4,5-dihydroxy-6-oxo-
7-(phosphooxy)heptanoate synthase (NCBI-
OFX87022) from Bacteroidetes bacterium
GWE2_32_14>
MSGKKHRLAKILDKKGGKSCIIPIDHGTTLGPMEG
IENSFKAISNFINGGASAIVLHKGILKMVSNYPEL
LKTNYLMHLSVSTCLGRSQSHKVIVGNVEEAIRLG
AIGISIHTNLGGEYETEMIKDLGRIAEECYKWEMP
LLSMIYVDNEKENPQKIAHAARLAQELGADIVKVD
YPGTIEGFKKVLNGVQIPVLIAGGGKSDNPKTFLK
WNDAM KAGASGISAGRNIFQYEYPELLTRIICNL
IEGKWELDECFKHLNGELVKMK
<4; Protein/1;3-amino-4-benzoic acid
synthase (A0JC76) from Streptomyces
griseus>
MSSSPSPSPSSSSSSSASSSASSSPSSSSKLTWLD
IRSVGEARAAIVQEALHHRVEALVADDPAHLADLP
PTVAKVLLWGKQIPEEFGEATVVVVDPSKHGVTPA
ELALKHPEIEFGRFVEIIDAPTLEDACESSRTEKW
SVLLFRDPTKIPLEIVIAAAARASGSMVTIAQDLE
EAEILFGVLEHGSDGVMMAPKTVGDAAELKRIAEA
GIPNLNLTELRVVETSHIGMGERACVDTTTHFGED
EGILVGSHSKGMILCVSETHPLPYMPTRPFRVNAG
AIHSYTLGRDERTNYLSELKTGSKLTAVDIKGNTR
LVTVGRVKIETRPLISIDAEAPDGRRVNLILQDDW
HVRVLGPGGTVLNSTELKPGDTVLGYLPVEDRHVG
YPINEFCLEK
<5; Protein/1;2-amino-4,5-dihydroxy-
6-oxo-7-(phosphooxy)heptanoate
synthase (A0A132MRF8) from
Streptomyces thermoautotrophicus>
MPFNNSFARQVRLRRLYRHGDDRLLVVPLDHSVTD
GPITGGSRLNHLVGQLAANGVDAVVLHKGSLRYID
GRWFTQTALIVHLSASTKHAPDPNAKYLVASVEEA
LRLGADAVSVHVNLGSQDERQQIADLAAVSEACDR
WNVPLLAMMYPRGPKIENPRDPALVAHAASLAADL
GADIVKTVYTGSAAEMAEITQNCPVPIVVAGGPRL
DSAEAVLSYVDDALKAGAAGVAMGRNVFQAPDPGA
MARRLVDLIHAGQTPSLEPDIESLQLATK
<6; Protein/1;3-amino-4-benzoic acid
synthase (W5WBR4) from Kutzneria
aibida DSM 43870>
MKFAWIDLRSTADTQLEAVVAAAVHARVQGVVSDR
PEVLASLPPTVTKVLLPAQPVADAQVDLVTTVLTD
ADQLDRLVAENHSGAVFVEVADDRTLKLACAAAVA
LPYTVVSFVDPTKIPLEIVIAAADRAQGKLICVVA
DLEEATIVLDVLEHGSDGVMLAPRDATEVFALARL
LEAGTQDLALSTLVVEGIEHNGLGDRVCVDTCSHF
EEDEGILVGSYSSGFILCCSETHPLPYMPTRPFRV
NAGALHSYVLGPDNRTNYLSELKSGSVTLAVNAEG
RTRRVVVGRAKLESRPLLTITAHSPEGVKVSLTVQ
DDWHVRVLGPGGKVRNVTELQAGDELLGYIATDKR
HVGIPIGEFCKES
<7; Protein/1;2-amino-4,5-dihydroxy-
6-oxo-7-(phosphooxy)heptanoate
synthase (NCBI-CUB39904.1)
from Bacillus cereus>
MSGKQLRLSRIINSDTNKACIVPIDHGTTLGPIKG
LPDYIELINSLIMGGTDGIVLH KGILSRVGGYPH
LSKGTYLAHLSVSTILNSDSTHKVLVCTVEEAIKH
GADGISVHINIGSEYESEMIKKLGDVSKACNEWGM
PLLAMMYSHKTPKDSFHISHVARIGEELGADIIKV
DYPGSIEDMEMITKSVQAPVLIAGGSNKNDDAALL
SLVNDALIGGAAGISIGRNIFQHDDPAYITNLVSS
LVHGRLSFNECLERIENYKLSIL
<8; Protein/1;3-dehydroquinate synthase
(A0A1T3V8D3) from Bacillus anthracis>
MKTRPIWYDARNLKDEKSTLPFVLTSPIDYVLFSK
AQVKNINLPKKTQVIIEIHKMDDIKELPKENIVLS
HDMKLLEDVKNLGYQTALYRKIVPETDLESVWQEG
LTFNYLVVELTDETNIPLELLIARLQDKSTNLIKV
VKNYQDMEVSIGVMEVGSDGVLLKTEDIQELVKVN
DYITNSKQSSIKMTKGKVVEVEHIGMGSRACIDTT
DLLKTNEGMIVGSTSSGGLLVSSETHFLPYMDLRP
FRVNAGAVHSYVWAPNNMTSYITELKAGSKVLVVD
TEGNTREISVGRVKIEVRPLLKIAVEVNGEIINTI
VQDDWHIRIFGANGEPRNASTIKVGEELLVYSCTS
GRHVGIKIEEQILEV
<9; Protein/1;2-amino-4,5-dihydroxy-
6-oxo-7-(phosphooxy)heptanoate
synthase (A0JC77) from
Streptomyces griseus>
MAPNAPFARSLRLQRLHHHDPDRLFIVPLDHSITD
GPLSRAHRLDPLVGELASHHVDGIVLHKGSLRHVD
PEWFTRTSLIVHLSASTVHAPDPNAKYLVSSVEES
LRMGADAVSVHVNLGSEGERHQIADMAAVAEACDR
WNVPLLAMMYPRGPKIDDPRDPALVAHAVQVAVDL
GADLVKTLYVGSVAAMAEITAASPVPVVVVGGPRD
SDESRILAYVDDALRGGAAGVAMGRNVFQAPDPGA
MAD KLSDLIHN SGTRGAARAPAGAAAGAA
<10; Protein/1;3-amino-4-benzoic acid
synthase (A0A0K2YDP9) from
Rhodococcus sp. RD6.2>
MKGTRPNMDSTIVTDQIVPTAGAGADSRRTPVSRE
GHFAWLDVRAVDEDLLPAVVQAALHHRIDGILSDD
VATFEGLPPTVRRILAVDAPVAEDFPYDAVDLVIL
SNRGDVHSKHIGHTPDRGVHVVVSDAPTLQEACEV
VREVPWTLLTFTDPTKIPLEIVIAAAENSGGRTIT
TVNDVEDAEIVKLVLEHGSDGLLLAPRTADDVVKL
ARIVDHKLEGMELSELVVTKVEHIGMGDRACIDTC
SLLELDEGCLIGSFSTGMFLSCSETHPLPYMPTRP
FRWNAGAVHSYVLGPDNRTRYVSELQAGFPILAVR
TDGSVREVRIGRVKIEKRPLISITAVAPNGKNVNV
IAQDDWHVRLLGPGGSVNNVTELTPGDVLLGYVPT
EARHVGLPITEFCDER
<11; Protein/1;2-amino-4,5-dihydroxy-6-
oxo-7-(phosphooxy) heptanoate synthase
(A0A0M4DD67) from Streptomyces
pristinaespiralis>
MTSYGHFARSLRLRRLYRHSTAGLMITPLDHSISD
GPVVPKGTTLDHLAGRLAAGGSDAVVVHKGSVRHI
SPERFAAMSLIIHLNASTSRALDPNAKYVVAGVEE
ALRLGADAVSVHVNLGSDDEREQIGDLGRIADACD
RWNLPLLAMVYPRGPRITDPRDPEMVAHAVTIAAD
LGADLVKTVFLGSTAEMLDLTAACPVPVLVAGGPA
LDKEEDVLAYVRDALAGGAGGVAMGRNIFQAADPR
RLAAKVARLVHHFPEQHFGTGPFAGGDARLDGERL
TPHHLDDTHLDPAHPDHPRLDDSRLDVSTGGPHHD
GRQTVLA
<12; Protein/1;2-amino-4,5-dihydroxy-
6-oxo-7-(phosphooxy) heptanoate (K0KAK8)
synthase from Saccharothrix
espanaensis ATCC 51144>
MRKALRLRRLSRPRDDKYLFVPLDHSVSDGPVVPR
DRWHDLIRSVVVGGADAIIVHKGRVRTLDPQVLGG
CALVVHLSAGTSHAADANAKVLVGEVEEALRLGAD
AVSVHVNIGSDTEERQLVDFGVVANACDSWNVPLV
AMVYPRGPRIADPSDPALLAHVVNIAVDLGADLVK
TNLALPVERMAEVVASCPIPVLVAGGPATGGSLVD
HARASMATGCAGLAVGRRVFTSPAPMTLVAELASV
IHADHPAELVHAMTGAVS
<13; Protein/1;3-amino-4-benzoic acid
synthase (A0A0K6LJE3) from
Bacillus cereus>
MMKTRPIWYDARNLKDEKSTLPFVLTSPIDYVLFS
KAQVKNINLPKKTQVIIEIHKMDDIKELPKENIVL
SHDMKLLEDVKNLGYQTALYRKIVPETDLESVWQE
GLTFNYLVVELTDETNIPLELLIARLQDKSTNLIK
VVKNYQDMEVSIGVMEVGSDGVLLKTEDIQELVKV
NDYITNSKQSSIKMTKGKVVEVEHIGMGSRACIDT
TDLLKTNEGMIVGSTSSGGLLVSSETHFLPYMDLR
PFRVNAGAVHSYVWAPNNMTSYITELKAGSKVLVV
DTEGNTREISVGRVKIEVRPLLKIAVEVNGEIINT
IVQDDWHIRIFGANGEPRNASTIKVGEELLVYSCT
SGRHVGIKIEEQILEV
<14; Protein/1;Aspartokinase (P26512)
from Corynebacterium glutamicum
ATCC 13032>
MALVVQKYGGSSLESAERIRNVAERIVATKKAGND
VVVVCSAMGDTTDELLELAAAVNPVPPAREMDMLL
TAGERISNALVAMAIESLGAEAQSFTGSQAGVLTT
ERHGNARIVDVTPGRVREALDEGKICIVAGFQGVN
KETRDVTTLGRGGSDTTAVALAAALNADVCEIYSD
VDGVYTADPRIVPNAQKLEKLSFEEMLELAAVGSK
ILVLRSVEYARAFNVPLRVRSSYSNDPGTLIAGSM
EDIPVEEAVLTGVATDKSEAKVTVLGISDKPGEAA
KVFRALADAEINIDMVLQNVSSVEDGTTDITFTCP
RSDGRRAMEILKKLQVQGNWTNVLYDDQVGKVSLV
GAGMKSHPGVTAEFMEALRDVNVNIELISTSEIRI
SVLIREDDLDAAARALHEQFQLGGEDEAVVYAGTG
R

What is claimed is:

1. An engineered microbial cell that produces 3-amino-4-hydroxybenzoic acid, wherein the engineered microbial cell expresses:
(a) a non-native 2-amino-4,5-dihydroxy-6-oxo-7-(phosphooxy) heptanoate synthase; and
(b) a non-native 3-amino-4-benzoic acid synthase.

2. The engineered microbial cell of claim 1, that comprises increased activity of at least one or more upstream pathway enzyme(s) leading to:
(a) L-aspartate semi-aldehyde; and/or
(b) dihydroxyacetone phosphate (DHAP), said increased activity being increased relative to a control cell.

3. The engineered microbial cell of claim 2, wherein the engineered microbial cell comprises increased activity of at least one or more upstream pathway enzyme(s) leading to L-aspartate semi-aldehyde.

4. The engineered microbial cell of claim 3, wherein the one or more upstream pathway enzyme(s) are selected from the group consisting of aspartate semi-aldehyde dehydrogenase, apartokinase, aspartate aminotransferase, pyruvate carboxylase, phosphoenolpyruvate (PEP) carboxylase, PEP synthase, malate dehydrogenase, glutamate dehydrogenase, glutamate synthase, and glutamine synthetase.

5. The engineered microbial cell of claim 2, wherein the engineered microbial cell comprises increased activity of at least one or more upstream pathway enzyme(s) leading to DHAP.

6. The engineered microbial cell of claim 5, wherein the one or more upstream pathway enzyme(s) comprise aldolase.

7. The engineered microbial cell of any one of claims 2-6, wherein the activity of the one or more upstream pathway enzyme(s) is increased by expressing an enzyme variant that has increased cytosolic localization, relative to that of the native enzyme.

8. The engineered microbial cell of claim 7, wherein the enzyme variant has a C-terminal truncation relative to the native enzyme.

9. The engineered microbial cell of claim 7 or claim 8, wherein the enzyme variant comprises a variant of an enzyme selected from the group consisting of aspartate aminotransferase, pyruvate carboxylase, phosphoenolpyruvate (PEP) carboxylase, PEP synthase, malate dehydrogenase, and combinations thereof.

10. The engineered microbial cell of any one of claims 2-9, wherein the activity of the one or more upstream pathway enzyme(s) is increased by expressing one or more feedback-deregulated enzyme(s).

11. The engineered microbial cell of claim 10, where the one or more feedback-deregulated enzyme(s) are selected from the group consisting of a feedback-deregulated aspartate kinase, a feedback-deregulated aspartate semi-aldehyde dehydrogenase, and a feedback-deregulated pyruvate carboxylase.

12. The engineered microbial cell of claim 11, where the one or more feedback-deregulated enzyme(s) are selected from the group consisting of:
(a) a feedback-deregulated Corynebacterium glutamicum ATCC 13032 aspartate kinase (UniProt ID P26512) comprising the amino acid substitution Q298G;
(b) a feedback-deregulated aspartate-semialdehyde dehydrogenase (EC 1.2.1.11) comprising the amino acid substitutions D66G, S202F, R234H, D272E, and K285E; and
(c) a feedback-deregulated pyruvate carboxylase (EC 6.4.1.1) comprising the amino acid substitution P458S.

13. The engineered microbial cell of claim 12, wherein the one or more feedback-deregulated enzyme(s) comprise a feedback-deregulated Corynebacterium glutamicum ATCC 13032 aspartate kinase (UniProt ID P26512) comprising the amino acid substitution Q298G.

14. The engineered microbial cell of any one of claims 1-13, wherein the engineered microbial cell comprises reduced activity of one or more protein(s) that reduce the concentration of one or more upstream pathway precursor(s), said reduced activity being reduced relative to a control cell.

15. The engineered microbial cell of claim 14, wherein the one or more upstream precursor(s) comprise L-aspartate semi-aldehyde and/or dihydroxyacetone phosphate (DHAP).

16. The engineered microbial cell of claim 15, wherein the one or more upstream precursor(s) comprise L-aspartate semi-aldehyde.

17. The engineered microbial cell of claim 16, wherein the one or more protein(s) that reduce the concentration of L-aspartate semi-aldehyde are selected from the group consisting of homoserine dehydrogenase, 4-hydroxy-tetrahydrodipicolinate synthase, and phosphoenolpyruvate (PEP) carboxykinase.

18. The engineered microbial cell of claim 15, wherein the one or more upstream precursor(s) comprise DHAP.

19. The engineered microbial cell of claim 18, wherein the one or more protein(s) that reduce the concentration of DHAP are selected from the group consisting of glycerol-3-phosphate dehydrogenase, Saccharomyces cerevisiae FPS1 and its orthologs, triose phosphate isomerase, glycerol-3-phosphate/dihydroxyacetone phosphate acyltransferase, and pyruvate dehydrogenase.

20. The engineered microbial cell of any one of claims 14-19, wherein the reduced activity is achieved by one or more means selected from the group consisting of gene deletion, gene disruption, altering regulation of a gene, replacing a native promoter with a less active promoter; and expression of a protein variant having reduces activity.

21. The engineered microbial cell of any one of claims 1-20, wherein the engineered microbial cell comprises increased activity of one or more enzyme(s) that increase the supply of the reduced form of nicotinamide adenine dinucleotide phosphate (NADPH), said increased activity being increased relative to a control cell.

22. The engineered microbial cell of claim 21, wherein the one or more enzyme(s) that increase the supply of the reduced form of NADPH are selected from the group consisting of pentose phosphate pathway enzymes, NADP+-dependent glyceraldehyde 3-phosphate dehydrogenase (GAPDH), and NADP+-dependent glutamate dehydrogenase.

23. The engineered microbial cell of any one of claims 1-22, wherein the engineered microbial cell comprises altered cofactor specificity of one or more upstream pathway enzyme(s) from the reduced form of nicotinamide adenine dinucleotide phosphate (NADPH) to the reduced from of nicotinamide adenine dinucleotide (NADH).

24. The engineered microbial cell of claim 23, wherein the one or more upstream pathway enzyme(s) whose cofactor specificity is altered comprise aspartate semi-aldehyde dehydrogenase.

25. The engineered microbial cell of any one of claims 1-24, wherein:
(a) the non-native 2-amino-4,5-dihydroxy-6-oxo-7-(phosphooxy) heptanoate synthase has at least 70% amino acid sequence identity with a Streptomyces sp. Root63 2-amino-4,5-dihydroxy-6-oxo-7-(phosphooxy) heptanoate synthase comprising SEQ ID NO:1; and
(b) the non-native 3-amino-4-benzoic acid synthase has at least 70% amino acid sequence identity with a Saccharothrix espanaensis ATCC 51144 3-amino-4-benzoic acid synthase comprising SEQ ID NO:2.

26. The engineered microbial cell of claim 25, wherein:
(a) the non-native 2-amino-4,5-dihydroxy-6-oxo-7-(phosphooxy) heptanoate synthase comprises SEQ ID NO:1; and
(b) the non-native 3-amino-4-benzoic acid synthase comprises SEQ ID NO:2.

27. The engineered microbial cell of claim 25 or claim 26, wherein the engineered microbial cell is a bacterial cell.

28. The engineered microbial cell of claim 27, wherein the bacterial cell is a Corynebacteria glutamicum cell.

29. The engineered microbial cell of claim 25 or claim 26, wherein the engineered microbial cell comprises a yeast cell.

30. The engineered microbial cell of claim 29, wherein the yeast cell is a Saccharomyces cerevisiae cell.

31. The engineered microbial cell of claim 30, wherein:
(a) the non-native 2-amino-4,5-dihydroxy-6-oxo-7-(phosphooxy) heptanoate synthase has at least 70% amino acid sequence identity with a Streptomyces thermoautotrophicus 2-amino-4,5-dihydroxy-6-oxo-7-(phosphooxy) heptanoate synthase comprising SEQ ID NO:5; and
(b) the non-native 3-amino-4-benzoic acid synthase has at least 70% amino acid sequence identity with a Streptomyces griseus 3-amino-4-benzoic acid synthase comprising SEQ ID NO:4.

32. The engineered microbial cell of claim 31, wherein:
(a) the non-native 2-amino-4,5-dihydroxy-6-oxo-7-(phosphooxy) heptanoate synthase comprises SEQ ID NO:5; and
(b) the non-native 3-amino-4-benzoic acid synthase comprises SEQ ID NO:4.

33. The engineered microbial cell of any one of claims 1-26, wherein, when cultured, the engineered microbial cell produces 3-amino-4-hydroxybenzoic acid at a level of at least 20 μg/L of culture medium or at a level of at least 4 mg/L of culture medium.

34. A culture of engineered microbial cells according to any one of claims 1-33.

35. A method of culturing engineered microbial cells according to any one of claims 1-33, the method comprising culturing the cells under conditions suitable for producing 3-amino-4-hydroxybenzoic acid, optionally wherein the method additionally comprises recovering 3-amino-4-hydroxybenzoic acid from the culture.


SEQUENCE LISTING (14 sequences)

SEQ ID NO: 1 - protein, 275 aa, Streptomyces sp. Root63
MTLNGSFGARLRGRKLYRNSPDRLLVVPLDHSVTDGPIATAAGLNHLVGRLSDNGVDAIVLHKGTLRRLDPEPFTRTSLIVHLSVSTMHAPDPDAKYLVSDVENALRLGADAVSVHVNVGSAGEAAQVADMAAVADACDRWNIPLLAMMYPRGPEISDPRDLVLVKHVATLAADLGADLVKVPCPRKVTDLADVVSACPVPVLVAGGQVAGTTEELLDAVGGILDTGVGGLAMGRNIFQADDPGKRARQVADLVHAPPLRYGPAGTPGPAPHRLP

SEQ ID NO: 2 - protein, 397 aa, Saccharothrix espanaensis ATCC 51144
MPPGHRIRPDRRASPHWNAVGGWFGVKFAWIDIRAVDARHREAVVDAAVHAGLGGVLDHRLETLATLPPTVTKVLLPGPGEVPPAEAAGACDVVLTRVATFTELDKLKLVAGEVDAHAGAFVEVVDDLTLRVACAAVQALEHTVVRFRDPTKIPLEIVIAAADRAPGLLVCEADGIEEARIVLDVLEKGSDGLLVAPRDANDVFDVDKLLRTATPDLALTTLTVRSVEHNGLGDRICVDTCTHFREDEGILVGSFAHGFVLCVSETHPLPYMPTRPFRVNAGALHSYVFGADNRTNYLSELKAGSTVLGVTADGRTRRIVVGRVKLESRPLLTVHATAPDGTEVALTLQDDWHVRVLGPGAAVLNSTELEPGDQLLGYLATDKRHVGWPVGEFCIEK

SEQ ID NO: 3 - protein, 267 aa, Bacteroidetes bacterium GWE2_32_14
MSGKKHRLAKILDKKGGKSCIIPIDHGTTLGPMEGIENSFKAISNFINGGASAIVLHKGILKMVSNYPELLKTNYLMHLSVSTCLGRSQSHKVIVGNVEEAIRLGAIGISIHTNLGGEYETEMIKDLGRIAEECYKWEMPLLSMIYVDNEKENPQKIAHAARLAQELGADIVKVDYPGTIEGFKKVLNGVQIPVLIAGGGKSDNPKTFLKVVNDAMKAGASGISAGRNIFQYEYPELLTRIICNLIEGKWELDECFKHLNGELVKMK

SEQ ID NO: 4 - protein, 396 aa, Streptomyces griseus
MSSSPSPSPSSSSSSSASSSASSSPSSSSKLTWLDIRSVGEARAAIVQEALHHRVEALVADDPAHLADLPPTVAKVLLVVGKQIPEEFGEATVVVVDPSKHGVTPAELALKHPEIEFGRFVEIIDAPTLEDACESSRTEKWSVLLFRDPTKIPLEIVIAAAARASGSMVTIAQDLEEAEILFGVLEHGSDGVMMAPKTVGDAAELKRIAEAGIPNLNLTELRVVETSHIGMGERACVDTTTHFGEDEGILVGSHSKGMILCVSETHPLPYMPTRPFRVNAGAIHSYTLGRDERTNYLSELKTGSKLTAVDIKGNTRLVTVGRVKIETRPLISIDAEAPDGRRVNLILQDDWHVRVLGPGGTVLNSTELKPGDTVLGYLPVEDRHVGYPINEFCLEK

SEQ ID NO: 5 - protein, 274 aa, Streptomyces thermoautotrophicus
MPFNNSFARQVRLRRLYRHGDDRLLVVPLDHSVTDGPITGGSRLNHLVGQLAANGVDAVVLHKGSLRYIDGRWFTQTALIVHLSASTKHAPDPNAKYLVASVEEALRLGADAVSVHVNLGSQDERQQIADLAAVSEACDRWNVPLLAMMYPRGPKIENPRDPALVAHAASLAADLGADIVKTVYTGSAAEMAEITQNCPVPIVVAGGPRLDSAEAVLSYVDDALKAGAAGVAMGRNVFQAPDPGAMARRLVDLIHAGQTPSLEPDIESLQLATK

SEQ ID NO: 6 - protein, 363 aa, Kutzneria albida DSM 43870
MKFAWIDLRSTADTQLEAVVAAAVHARVQGVVSDRPEVLASLPPTVTKVLLPAQPVADAQVDLVTTVLTDADQLDRLVAENHSGAVFVEVADDRTLKLACAAAVALPYTVVSFVDPTKIPLEIVIAAADRAQGKLICVVADLEEATIVLDVLEHGSDGVMLAPRDATEVFALARLLEAGTQDLALSTLVVEGIEHNGLGDRVCVDTCSHFEEDEGILVGSYSSGFILCCSETHPLPYMPTRPFRVNAGALHSYVLGPDNRTNYLSELKSGSVTLAVNAEGRTRRVVVGRAKLESRPLLTITAHSPEGVKVSLTVQDDWHVRVLGPGGKVRNVTELQAGDELLGYIATDKRHVGIPIGEFCKES

SEQ ID NO: 7 - protein, 267 aa, Bacillus cereus
MSGKQLRLSRIINSDTNKACIVPIDHGTTLGPIKGLPDYIELINSLIMGGTDGIVLHKGILSRVGGYPHLSKGTYLAHLSVSTILNSDSTHKVLVCTVEEAIKHGADGISVHINIGSEYESEMIKKLGDVSKACNEWGMPLLAMMYSHKTPKDSFHISHVARIGEELGADIIKVDYPGSIEDMEMITKSVQAPVLIAGGSNKNDDAALLSLVNDALIGGAAGISIGRNIFQHDDPAYITNLVSSLVHGRLSFNECLERIENYKLSIL

SEQ ID NO: 8 - protein, 365 aa, Bacillus anthracis
MKTRPIWYDARNLKDEKSTLPFVLTSPIDYVLFSKAQVKNINLPKKTQVIIEIHKMDDIKELPKENIVLSHDMKLLEDVKNLGYQTALYRKIVPETDLESVWQEGLTFNYLVVELTDETNIPLELLIARLQDKSTNLIKVVKNYQDMEVSIGVMEVGSDGVLLKTEDIQELVKVNDYITNSKQSSIKMTKGKVVEVEHIGMGSRACIDTTDLLKTNEGMIVGSTSSGGLLVSSETHFLPYMDLRPFRVNAGAVHSYVWAPNNMTSYITELKAGSKVLVVDTEGNTREISVGRVKIEVRPLLKIAVEVNGEIINTIVQDDWHIRIFGANGEPRNASTIKVGEELLVYSCTSGRHVGIKIEEQILEV

SEQ ID NO: 9 - protein, 274 aa, Streptomyces griseus [sequence omitted]

SEQ ID NO: 10 - protein, 401 aa, Rhodococcus sp. RD6.2
MKGTRPNMDSTIVTDQIVPTAGAGADSRRTPVSREGHFAWLDVRAVDEDLLPAVVQAALHHRIDGILSDDVATFEGLPPTVRRILAVDAPVAEDFPYDAVDLVILSNRGDVHSKHIGHTPDRGVHVVVSDAPTLQEACEVVREVPWTLLTFTDPTKIPLEIVIAAAENSGGRTITTVNDVEDAEIVKLVLEHGSDGLLLAPRTADDVVKLARIVDHKLEGMELSELVVTKVEHIGMGDRACIDTCSLLELDEGCLIGSFSTGMFLSCSETHPLPYMPTRPFRWNAGAVHSYVLGPDNRTRYVSELQAGFPILAVRTDGSVREVRIGRVKIEKRPLISITAVAPNGKNVNVIAQDDWHVRLLGPGGSVNNVTELTPGDVLLGYVPTEARHVGLPITEFCDER

SEQ ID NO: 11 - protein, 322 aa, Streptomyces pristinaespiralis
MTSYGHFARSLRLRRLYRHSTAGLMITPLDHSISDGPVVPKGTTLDHLAGRLAAGGSDAVVVHKGSVRHISPERFAAMSLIIHLNASTSRALDPNAKYVVAGVEEALRLGADAVSVHVNLGSDDEREQIGDLGRIADACDRWNLPLLAMVYPRGPRITDPRDPEMVAHAVTIAADLGADLVKTVFLGSTAEMLDLTAACPVPVLVAGGPALDKEEDVLAYVRDALAGGAGGVAMGRNIFQAADPRRLAAKVARLVHHFPEQHFGTGPFAGGDARLDGERLTPHHLDDTHLDPAHPDHPRLDDSRLDVSTGGPHHDGRQTVLA

SEQ ID NO: 12 - protein, 263 aa, Saccharothrix espanaensis ATCC 51144
MRKALRLRRLSRPRDDKYLFVPLDHSVSDGPVVPRDRWHDLIRSVVVGGADAIIVHKGRVRTLDPQVLGGCALVVHLSAGTSHAADANAKVLVGEVEEALRLGADAVSVHVNIGSDTEERQLVDFGVVANACDSWNVPLVAMVYPRGPRIADPSDPALLAHVVNIAVDLGADLVKTNLALPVERMAEVVASCPIPVLVAGGPATGGSLVDHARASMATGCAGLAVGRRVFTSPAPMTLVAELASVIHADHPAELVHAMTGAVS

SEQ ID NO: 13 - protein, 366 aa, Bacillus cereus
MMKTRPIWYDARNLKDEKSTLPFVLTSPIDYVLFSKAQVKNINLPKKTQVIIEIHKMDDIKELPKENIVLSHDMKLLEDVKNLGYQTALYRKIVPETDLESVWQEGLTFNYLVVELTDETNIPLELLIARLQDKSTNLIKVVKNYQDMEVSIGVMEVGSDGVLLKTEDIQELVKVNDYITNSKQSSIKMTKGKVVEVEHIGMGSRACIDTTDLLKTNEGMIVGSTSSGGLLVSSETHFLPYMDLRPFRVNAGAVHSYVWAPNNMTSYITELKAGSKVLVVDTEGNTREISVGRVKIEVRPLLKIAVEVNGEIINTIVQDDWHIRIFGANGEPRNASTIKVGEELLVYSCTSGRHVGIKIEEQILEV

SEQ ID NO: 14 - protein, 421 aa, Corynebacterium glutamicum ATCC 13032
MALVVQKYGGSSLESAERIRNVAERIVATKKAGNDVVVVCSAMGDTTDELLELAAAVNPVPPAREMDMLLTAGERISNALVAMAIESLGAEAQSFTGSQAGVLTTERHGNARIVDVTPGRVREALDEGKICIVAGFQGVNKETRDVTTLGRGGSDTTAVALAAALNADVCEIYSDVDGVYTADPRIVPNAQKLEKLSFEEMLELAAVGSKILVLRSVEYARAFNVPLRVRSSYSNDPGTLIAGSMEDIPVEEAVLTGVATDKSEAKVTVLGISDKPGEAAKVFRALADAEINIDMVLQNVSSVEDGTTDITFTCPRSDGRRAMEILKKLQVQGNWTNVLYDDQVGKVSLVGAGMKSHPGVTAEFMEALRDVNVNIELISTSEIRISVLIREDDLDAAARALHEQFQLGGEDEAVVYAGTGR